(19) United States
(12) Patent Application Publication
(10) Pub. No.: US 2023/0073363 A1
(43) Pub. Date: Mar. 9, 2023

(54) User Interface Solutions for Providing Sub-Perception Stimulation in an Implantable Stimulator System

(71) Applicant: Boston Scientific Neuromodulation Corporation, Valencia, CA (US)

(72) Inventors: Ismael Huertas Fernandez, Sevilla (ES); Ewan Gillespie, Stevenson Ranch, CA (US)

(21) Appl. No.: 17/820,467

(22) Filed: Aug. 17, 2022

Related U.S. Application Data
(60) Provisional application No. 63/236,432, filed on Aug. 24, 2021.

(51) Int. Cl. A61N 1/372; A61N 1/36

(52) U.S. Cl. CPC A61N 1/37247; A61N 1/36175; A61N 1/36171; A61N 1/36062; A61N 1/36071

(57) ABSTRACT

Graphical User Interface (GUI) control of a stimulator device is disclosed. The GUI receives modeling information indicating optimal stimulation parameters for a patient based on patient testing, and may also receive an indication of a particular stimulation mode to be used for the patient which comprises a subset of those parameters. The GUI provides simple options to allow a user to navigate the optimal parameters or subsets to constrain selection to only those stimulation parameters set within the optimal parameters or subsets.

### CROSS REFERENCE TO RELATED APPLICATIONS

This application is a non-provisional of U.S. Provisional Patent Application Ser. No. 63/236,432, filed Aug. 24, 2021, which is incorporated herein by reference in its entirety, and to which priority is claimed.

### FIELD OF THE INVENTION

This application relates to Implantable Medical Devices (IMDs) generally, Spinal Cord Stimulators more specifically, and to methods of control of such devices.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability with any implantable neurostimulator device system.

An SCS system typically includes an Implantable Pulse Generator (IPG) 10 shown in FIG. 1. The IPG 10 includes a biocompatible device case 12 that holds the circuitry and battery 14 necessary for the IPG to function. The IPG 10 is coupled to electrodes 16 via one or more electrode leads 15 that form an electrode array 17. The electrodes 16 are configured to contact a patient's tissue and are carried on a flexible body 18, which also houses the individual lead wires 20 coupled to each electrode 16. The lead wires 20 are also coupled to proximal contacts 22, which are insertable into lead connectors 24 fixed in a header 23 on the IPG 10, which header can comprise an epoxy for example. Once inserted, the proximal contacts 22 connect to header contacts within the lead connectors 24, which are in turn coupled by feedthrough pins through a case feedthrough to circuitry within the case 12, although these details aren't shown.

In the illustrated IPG 10, there are sixteen lead electrodes (E1-E16) split between two leads 15, with the header 23 containing a 2×1 array of lead connectors 24. However, the number of leads and electrodes in an IPG is application specific and therefore can vary. The conductive case 12 can also comprise an electrode (Ec). In a SCS application, the electrode leads 15 are typically implanted proximate to the dura in a patient's spinal column on the right and left sides of the spinal cord midline. The proximal electrodes 22 are tunneled through the patient's tissue to a distant location such as the buttocks where the IPG case 12 is implanted, at which point they are coupled to the lead connectors 24. In other IPG examples designed for implantation directly at a site requiring stimulation, the IPG can be lead-less, having electrodes 16 instead appearing on the body of the IPG for contacting the patient's tissue. The IPG leads 15 can be integrated with and permanently connected the case 12 in other IPG solutions. The goal of SCS therapy is to provide electrical stimulation from the electrodes 16 to alleviate a patient's symptoms, most notably chronic back pain.

IPG 10 can include an antenna 26a allowing it to communicate bi-directionally with a number of external devices, as shown in FIG. 4. The antenna 26a as depicted in FIG. 1 is shown as a conductive coil within the case 12, although the coil antenna 26a can also appear in the header 23. When antenna 26a is configured as a coil, communication with external devices preferably occurs using near-field magnetic induction. IPG may also include a Radio-Frequency (RF) antenna 26b. In FIG. 1, RF antenna 26b is shown within the header 23, but it may also be within the case 12. RF antenna 26b may comprise a patch, slot, or wire, and may operate as a monopole or dipole. RF antenna 26b preferably communicates using far-field electromagnetic waves. RF antenna 26b may operate in accordance with any number of known RF communication standards, such as Bluetooth, Zigbee, WiFi, MICS, and the like.

Stimulation in IPG 10 is typically provided by pulses, as shown in FIG. 2. Stimulation parameters typically include the amplitude of the pulses (A; whether current or voltage); the frequency (F) and pulse width (PW) of the pulses; the electrodes 16 (E) activated to provide such stimulation; and the polarity (P) of such active electrodes, i.e., whether active electrodes are to act as anodes (that source current to the tissue) or cathodes (that sink current from the tissue). These stimulation parameters taken together comprise a stimulation program that the IPG 10 can execute to provide therapeutic stimulation to a patient.

In the example of FIG. 2, electrode E5 has been selected as an anode, and thus provides pulses which source a positive current of amplitude +A to the tissue. Electrode E4 has been selected as a cathode, and thus provides pulses which sink a corresponding negative current of amplitude −A from the tissue. This is an example of bipolar stimulation, in which only two lead-based electrodes are used to provide stimulation to the tissue (one anode, one cathode). However, more than one electrode may act as an anode at a given time, and more than one electrode may act as a cathode at a given time (e.g., tripole stimulation, quadripole stimulation, etc.).

The pulses as shown in FIG. 2 are biphasic, comprising a first phase 30a, followed quickly thereafter by a second phase 30b of opposite polarity. As is known, use of a biphasic pulse is useful in active charge recovery. For example, each electrodes' current path to the tissue may include a serially-connected DC-blocking capacitor, see, e.g., U.S. Patent Application Publication 2016/0144183, which will charge during the first phase 30a and discharged (be recovered) during the second phase 30b. In the example shown, the first and second phases 30a and 30b have the same duration and amplitude (although opposite polarities), which ensures the same amount of charge during both phases. However, the second phase 30b may also be charged balance with the first phase 30a if the integral of the amplitude and durations of the two phases are equal in magnitude, as is well known. The width of each pulse, PW, is defined here as the duration of first pulse phase 30a, although pulse width could also refer to the total duration of the first and second pulse phases 30a and 30b as well. Note that an interphase period (IP) during which no stimulation is provided may be provided between the two phases 30a and 30b.

IPG 10 includes stimulation circuitry 28 that can be programmed to produce the stimulation pulses at the electrodes as defined by the stimulation program. Stimulation circuitry 28 can for example comprise the circuitry described in U.S. Patent Application Publications 2018/0071513 and 2018/0071520, or described in U.S. Pat. Nos. 8,606,362 and 8,620,436. These references are incorporated herein by reference.

FIG. 3 shows an external trial stimulation environment that may precede implantation of an IPG 10 in a patient. During external trial stimulation, stimulation can be tried on a prospective implant patient without going so far as to implant the IPG 10. Instead, one or more trial leads 15′ are implanted in the patient's tissue 32 at a target location 34, such as within the spinal column as explained earlier. The proximal ends of the trial lead(s) 15′ exit an incision 36 and are connected to an External Trial Stimulator (ETS) 40. The ETS 40 generally mimics operation of the IPG 10, and thus can provide stimulation pulses to the patient's tissue as explained above. See, e.g., U.S. Pat. No. 9,259,574, disclosing a design for an ETS. The ETS 40 is generally worn externally by the patient for a short while (e.g., two weeks), which allows the patient and his clinician to experiment with different stimulation parameters to try and find a stimulation program that alleviates the patient's symptoms (e.g., pain). If external trial stimulation proves successful, trial lead(s) 15′ are explanted, and a full IPG 10 and lead(s) 15 are implanted as described above; if unsuccessful, the trial lead(s) 15′ are simply explanted.

Like the IPG 10, the ETS 40 can include one or more antennas to enable bi-directional communications with external devices, explained further with respect to FIG. 4. Such antennas can include a near-field magnetic-induction coil antenna 42a, and/or a far-field RF antenna 42b, as described earlier. ETS 40 may also include stimulation circuitry 44 able to form the stimulation pulses in accordance with a stimulation program, which circuitry may be similar to or comprise the same stimulation circuitry 28 present in the IPG 10. ETS 40 may also include a battery (not shown) for operational power.

FIG. 4 shows various external devices that can wirelessly communicate data with the IPG 10 and the ETS 40, including a patient, hand-held external controller 45, and a clinician programmer 50. Both of devices 45 and 50 can be used to send a stimulation program to the IPG 10 or ETS 40—that is, to program their stimulation circuitries 28 and 44 to produce pulses with a desired shape and timing described earlier. Both devices 45 and 50 may also be used to adjust one or more stimulation parameters of a stimulation program that the IPG 10 or ETS 40 is currently executing. Devices 45 and 50 may also receive information from the IPG 10 or ETS 40, such as various status information, etc.

External controller 45 can be as described in U.S. Patent Application Publication 2015/0080982 for example, and may comprise either a dedicated controller configured to work with the IPG 10. External controller 45 may also comprise a general purpose mobile electronics device such as a mobile phone which has been programmed with a Medical Device Application (MDA) allowing it to work as a wireless controller for the IPG 10 or ETS 40, as described in U.S. Patent Application Publication 2015/0231402. External controller 45 includes a user interface, including means for entering commands (e.g., buttons or icons) and a display 46. The external controller 45's user interface enables a patient to adjust stimulation parameters, although it may have limited functionality when compared to the more-powerful clinician programmer 50, described shortly.

The external controller 45 can have one or more antennas capable of communicating with the IPG 10 and ETS 40. For example, the external controller 45 can have a near-field magnetic-induction coil antenna 47a capable of wirelessly communicating with the coil antenna 26a or 42a in the IPG 10 or ETS 40. The external controller 45 can also have a far-field RF antenna 47b capable of wirelessly communicating with the RF antenna 26b or 42b in the IPG 10 or ETS 40.

The external controller 45 can also have control circuitry 48 such as a microprocessor, microcomputer, an FPGA, other digital logic structures, etc., which is capable of executing instructions an electronic device. Control circuitry 48 can for example receive patient adjustments to stimulation parameters, and create a stimulation program to be wirelessly transmitted to the IPG 10 or ETS 40.

Clinician programmer 50 is described further in U.S. Patent Application Publication 2015/0360038, and is only briefly explained here. The clinician programmer 50 can comprise a computing device 51, such as a desktop, laptop, or notebook computer, a tablet, a mobile smart phone, a Personal Data Assistant (PDA)-type mobile computing device, etc. In FIG. 4, computing device 51 is shown as a laptop computer that includes typical computer user interface means such as a screen 52, a mouse, a keyboard, speakers, a stylus, a printer, etc., not all of which are shown for convenience. Also shown in FIG. 4 are accessory devices for the clinician programmer 50 that are usually specific to its operation as a stimulation controller, such as a communication “wand” 54, and a joystick 58, which are coupleable to suitable ports on the computing device 51, such as USB ports 59 for example.

The antenna used in the clinician programmer 50 to communicate with the IPG 10 or ETS 40 can depend on the type of antennas included in those devices. If the patient's IPG 10 or ETS 40 includes a coil antenna 26a or 42a, wand 54 can likewise include a coil antenna 56a to establish near-filed magnetic-induction communications at small distances. In this instance, the wand 54 may be affixed in close proximity to the patient, such as by placing the wand 54 in a belt or holster wearable by the patient and proximate to the patient's IPG 10 or ETS 40.

If the IPG 10 or ETS 40 includes an RF antenna 26b or 42b, the wand 54, the computing device 51, or both, can likewise include an RF antenna 56b to establish communication with the IPG 10 or ETS 40 at larger distances. (Wand 54 may not be necessary in this circumstance). The clinician programmer 50 can also establish communication with other devices and networks, such as the Internet, either wirelessly or via a wired link provided at an Ethernet or network port.

To program stimulation programs or parameters for the IPG 10 or ETS 40, the clinician interfaces with a clinician programmer graphical user interface (GUI) 64 provided on the display 52 of the computing device 51. As one skilled in the art understands, the GUI 64 can be rendered by execution of clinician programmer software 66 on the computing device 51, which software may be stored in the device's non-volatile memory 68. One skilled in the art will additionally recognize that execution of the clinician programmer software 66 in the computing device 51 can be facilitated by control circuitry 70 such as a microprocessor, microcomputer, an FPGA, other digital logic structures, etc., which is capable of executing programs in a computing device. Such control circuitry 70, in addition to executing the clinician programmer software 66 and rendering the GUI 64, can also enable communications via antennas 56a or 56b to communicate stimulation parameters chosen through the GUI 64 to the patient's IPG 10.

A portion of the GUI 64 is shown in one example in FIG. 5. One skilled in the art will understand that the particulars of the GUI 64 will depend on where clinician programmer software 66 is in its execution, which will depend on the GUI selections the clinician has made. FIG. 5 shows the GUI 64 at a point allowing for the setting of stimulation parameters for the patient and for their storage as a stimulation program. To the left a program interface 72 is shown, which as explained further in the '038 Publication allows for naming, loading and saving of stimulation programs for the patient. Shown to the right is a stimulation parameters interface 82, in which specific stimulation parameters (A, D, F, E, P) can be defined for a stimulation program. Values for stimulation parameters relating to the shape of the waveform (A; in this example, current), pulse width (PW), and frequency (F) are shown in a waveform parameter interface 84, including buttons the clinician can use to increase or decrease these values.

Stimulation parameters relating to the electrodes 16 (the electrodes E activated and their polarities P), are made adjustable in an electrode parameter interface 86. Electrode stimulation parameters are also visible and can be manipulated in a leads interface 92 that displays the leads 15 (or 15′) in generally their proper position with respect to each other, for example, on the left and right sides of the spinal column. A cursor 94 (or other selection means such as a mouse pointer) can be used to select a particular electrode in the leads interface 92. Buttons in the electrode parameter interface 86 allow the selected electrode (including the case electrode, Ec) to be designated as an anode, a cathode, or off. The electrode parameter interface 86 further allows the relative strength of anodic or cathodic current of the selected electrode to be specified in terms of a percentage, X. This is particularly useful if more than one electrode is to act as an anode or cathode at a given time, as explained in the '038 Publication. In accordance with the example waveforms shown in FIG. 2, as shown in the leads interface 92, electrode E5 has been selected as the only anode to source current, and this electrode receives X=100% of the specified anodic current, +A. Likewise, electrode E4 has been selected as the only cathode to sink current, and this electrode receives X=100% of that cathodic current, −A.

The GUI 64 as shown specifies only a pulse width PW of the first pulse phase 30a. The clinician programmer software 66 that runs and receives input from the GUI 64 will nonetheless ensure that the IPG 10 and ETS 40 are programmed to render the stimulation program as biphasic pulses if biphasic pulses are to be used. For example, the clinician programming software 66 can automatically determine durations and amplitudes for both of the pulse phases 30a and 30b (e.g., each having a duration of PW, and with opposite polarities +A and −A). An advanced menu 88 can also be used (among other things) to define the relative durations and amplitudes of the pulse phases 30a and 30b, and to allow for other more advance modifications, such as setting of a duty cycle (on/off time) for the stimulation pulses, and a ramp-up time over which stimulation reaches its programmed amplitude (A), etc. A mode menu 90 allows the clinician to choose different modes for determining stimulation parameters. For example, as described in the '038 Publication, mode menu 90 can be used to enable electronic trolling, which comprises an automated programming mode that performs current steering along the electrode array by moving the cathode in a bipolar fashion.

While GUI 64 is shown as operating in the clinician programmer 50, the user interface of the external controller 45 may provide similar functionality.

### SUMMARY

A system for controlling an implantable medical device of a patient is disclosed, which may comprise: a first external device for configured to program the implantable stimulator device, comprising: a graphical user interface; and control circuitry configured to: receive a model comprising a plurality of sets of stimulation parameters developed for the patient, each of the sets of stimulation parameters being executable by the implantable stimulator device to provide stimulation to the patient, provide on the graphical user interface an adjustment for each of the stimulation parameters, wherein a range within which each stimulation parameter can be adjusted is automatically constrained by the model, and wherein when one of the stimulation parameters is adjusted within its range to a value, the control circuitry is configured to automatically constrain the ranges within which the other stimulation parameters can be adjusted in accordance with the model and the value.

In one example, the first external device comprises a patient remote controller. In one example, the first external device comprises a clinician programmer. In one example, the stimulation parameters comprise stimulation amplitude, pulse width, and frequency. In one example, the one of the stimulation parameters comprises the frequency, and wherein the other of the stimulation parameters comprises the pulse width and the amplitude. In one example, the system further comprises a second external device, wherein the model is determined at the second external device and transmitted to the first external device. In one example, the control circuitry is further configured to provide on the graphical user interface an option to select a stimulation mode, wherein the range within which each stimulation parameter can be adjusted is automatically further constrained by a selected stimulation mode. In one example, at least one of the stimulation modes corresponds to an activity or posture of the patient. In one example, at least one of the stimulation modes corresponds to stimulation having a particular feeling for the patient. In one example, at least one of the stimulation modes comprises a low power mode.

A method for controlling an implantable medical device of a patient is disclosed, which may comprise: providing a model at a first external device, wherein the model comprises a plurality of sets of stimulation parameters developed for the patient, each of the sets of stimulation parameters being executable by the implantable stimulator device to provide stimulation to the patient; providing on a graphical user interface of the first external device an adjustment for each of the stimulation parameters, wherein a range within which each stimulation parameter can be adjusted is automatically constrained by the model; adjusting one of the stimulation parameters within its range to a value; and automatically constraining the ranges within which the other stimulation parameters can be adjusted in accordance with the model and the value.

In one example, the first external device comprises a patient remote controller. In one example, the second external device comprises a clinician programmer. In one example, the stimulation parameters comprise stimulation amplitude, pulse width, and frequency. In one example, the one of the stimulation parameters comprises the frequency, and wherein the other of the stimulation parameters comprises the pulse width and the amplitude. In one example, the method further comprises determining the model at a second external device and transmitting the model to the first external device. In one example, the graphical user interface further provides an option to select a stimulation mode, wherein the range within which each stimulation parameter can be adjusted is automatically further constrained by a selected stimulation mode. In one example, at least one of the stimulation modes corresponds to an activity or posture of the patient. In one example, at least one of the stimulation modes corresponds to stimulation having a particular feeling for the patient. In one example, at least one of the stimulation modes comprises a low power mode.

A system for controlling an implantable medical device of a patient is disclosed, which may comprise: an external device for configured to communicate with the implantable medical device, comprising: a graphical user interface; and control circuitry configured to: receive a model comprising a plurality of sets of stimulation parameters developed for the patient, provide on the graphical user interface an option to increment through at least some of the sets of stimulation parameters, wherein (a) sequential increments increase a first of the stimulation parameters until a maximum value for the first stimulation parameter is reached in accordance with the model, (b) thereafter a next increment increases a second of the stimulation parameters, and (c) thereafter sequential increments increase the first stimulation parameter until a maximum value for the first stimulation parameter is reached in accordance with the model.

In one example, in step (b) if a maximum of the second parameter is reached in accordance with the model, a third of the stimulation parameters is increased. In one example, between steps (b) and (c) the first stimulation parameter is set to a minimum value in accordance with the model. In one example, the first external device comprises a patient remote controller. In one example, the first external device comprises a clinician programmer. In one example, the stimulation parameters comprise stimulation amplitude, pulse width, and frequency. In one example, the first stimulation parameter comprises the amplitude, and the second of the stimulation parameters comprises the pulse width. In one example, the system further comprises a second external device, wherein the model is determined at the second external device and transmitted to the first external device. In one example, the control circuitry is further configured to provide on the graphical user interface an option to select a stimulation mode, wherein the selected mode limits the maximum value for the first stimulation parameter. In one example, at least one of the stimulation modes corresponds to one or more of: an activity or posture of the patient; stimulation having a particular feeling for the patient; or a low power mode.

A method for controlling an implantable medical device of a patient is disclosed, which may comprise: providing a model at a first external device, wherein the model comprises a plurality of sets of stimulation parameters developed for the patient, each of the sets of stimulation parameters being executable by the implantable stimulator device to provide stimulation to the patient; and providing on a graphical user interface of the first external device an option to increment through at least some of the sets of stimulation parameters, wherein (a) sequential increments increase a first of the stimulation parameters until a maximum value for the first stimulation parameter is reached in accordance with the model, (b) thereafter a next increment increases a second of the stimulation parameters, and (c) thereafter sequential increments increase the first stimulation parameter until a maximum value for the first stimulation parameter is reached in accordance with the model.

In one example, in step (b) if a maximum of the second parameter is reached in accordance with the model, a third of the stimulation parameters is increased. In one example, between steps (b) and (c) the first stimulation parameter is set to a minimum value in accordance with the model. In one example, the first external device comprises a patient remote controller. In one example, the first external device comprises a clinician programmer. In one example, the stimulation parameters comprise stimulation amplitude, pulse width, and frequency. In one example, the first stimulation parameter comprises the amplitude, and the second of the stimulation parameters comprises the pulse width. In one example, the method further comprises determining the model at a second external device and transmitting the model to the first external device. In one example, the graphical user interface further provides an option to select a stimulation mode, wherein the selected mode limits the maximum value for the first stimulation parameter. In one example, at least one of the stimulation modes corresponds to one or more of: an activity or posture of the patient; stimulation having a particular feeling for the patient; or a low power mode.

### BRIEF DESCRIPTION OF THE DRAWINGS

FIG. 1 shows an Implantable Pulse Generator (IPG) useable for Spinal Cord Stimulation (SCS), in accordance with the prior art.

FIG. 2 shows an example of stimulation pulses producible by the IPG, in accordance with the prior art.

FIG. 3 shows use of an External Trial Stimulator (ETS) useable to provide stimulation before implantation of an IPG, in accordance with the prior art.

FIG. 4 shows various external devices capable of communicating with and programming stimulation in an IPG and ETS, in accordance with the prior art.

FIG. 5 shows a Graphical User Interface (GUI) of a clinician programmer external device for setting or adjusting stimulation parameters, in accordance with the prior art.

FIG. 6 shows sweet spot searching to determine effective electrodes for a patient using a movable sub-perception bipole.

FIGS. 7A-7D show sweet spot searching to determine effective electrodes for a patient using a movable supra-perception bipole.

FIG. 8 shows stimulation circuitry useable in the IPG or ETS capable of providing Multiple Independent Current Control to independently set the current at each of the electrodes.

FIGS. 9A-9C show various results of the study as a function of stimulation frequency in the 1 kHz to 10 kHz frequency range, including average optimal pulse width (FIG. 9A), mean charge per second and optimal stimulation amplitude (FIG. 9B), and back pain scores (FIG. 9C).

FIGS. 10A-10C show further analysis of relationships between average optimal pulse width and frequency in the 1 kHz to 10 kHz frequency range, and identifies statistically-significant regions of optimization of these parameters.

FIG. 11A shows results of patients tested with sub-perception therapy at frequencies at or below 1 kHz, and shows optimal pulse width ranges determined at tested frequencies, and optimal pulse width v. frequency regions for sub-perception therapy.

FIG. 11B shows various modelled relationships between average optimal pulse width and frequency at or below 1 kHz.

FIG. 11C shows the duty of cycle of the optimal pulse widths as a function of frequencies at or below 1 kHz.

FIG. 11D shows the average battery current and battery discharge time at the optimal pulse widths as a function of frequencies at or below 1 kHz.

FIGS. 12A and 12B show the results of additional testing that verifies the frequency versus pulse width relationships presented earlier.

FIG. 13 shows a fitting module showing how the relationships and regions determined relating optimal pulse width and frequency (<10 kHz) can be used to set sub-perception stimulation parameters for an IPG or ETS.

FIGS. 14A and 14B show an analysis of optimal sub-perception stimulation parameters, including a model of energy (mean charge per second) versus frequency.

FIGS. 14C-14E show various algorithms by which the model of FIGS. 14A and 14B can be used to selected optimal sub-perception stimulation parameters.

FIG. 14F shows that the model of energy can be viewed from the perspective of other stimulation parameters besides frequency. For example, energy can be modeled versus pulse width.

FIG. 15 shows a model derived from patients showing a surface denoting optimal sub-perception values for frequency and pulse width, and further including the patients' perception threshold pth as measured at those frequencies and pulse widths.

FIGS. 16A and 16B show the perception threshold pth plotted versus pulse width for a number of patients, and shows how results can be curve fit.

FIG. 17 shows a graph of parameter Z versus pulse width for patients, where Z comprises an optimal amplitude A for patients expressed as a percentage of perception threshold pth (i.e., Z=A/pth).

FIGS. 18A-18F show an algorithm used to derive a range of optimal sub-perception stimulation parameters (e.g., F, PW, and A) for a patient using the modelling information of FIGS. 15-17, and using perception threshold measurements taken on the patient.

FIG. 19 shows use of the optimal stimulation parameters in a patient external controller, including a user interface that allows the patient to adjust stimulation within the range.

FIGS. 20A-20F show the effect of statistic variance in the modelling, leading to the result that determined optimal stimulation parameters for a patient may occupy a volume. User interfaces for the patient external controller are also shown to allow the patient to adjust stimulation within this volume.

FIGS. 21A-21J show various examples of use of a modulation function to adjust the charge provided to the patient as a function of time.

FIG. 22 shows a stimulation mode user interface, from which a patient may select different stimulation modes, resulting in providing stimulation, or allowing the patient to control stimulation, using different subsets of stimulation parameters determined using the optimal stimulation parameters.

FIGS. 23A-28B show examples of different subsets of stimulation parameters based on the patient's selection of different stimulation modes. Figures labeled A (e.g., 23A) show frequencies and pulse widths of a subset, while figures labeled B (e.g., 23B) show amplitudes and perception thresholds for that subset. These figures show that subsets of stimulation parameters corresponding to different stimulation modes may comprise parameters wholly constrained by (i.e., wholly within) the determined optimal stimulation parameters, or may comprise parameters only partially constrained by the optimal stimulation parameters.

FIG. 29 shows an automatic mode in which the IPG and/or external controller are used to determine when particular stimulation modes should automatically be entered based on sensed information.

FIG. 30 shows another example of a simulation mode user interface, in which stimulation modes are presented for selection on a two-dimensional representation of stimulation parameters, although a three-dimensional representation indicative of subset volume can also be used.

FIG. 31 shows GUI aspects that allows a patient to adjust stimulation, where a suggested stimulation region for the patient is shown in conjunction with adjustment aspects.

FIG. 32 summarizes the modelling described earlier for determining optimal sub-perception stimulation parameters and subsets of those parameters.

FIGS. 33A-33E show GUI aspects for allowing a user to navigate stimulation parameters sets within the optimal stimulation parameters and/or subsets.

FIGS. 34A-34C show use of a neural dose adjustment interface useable in the GUI for allowing a user to navigate stimulation parameters sets within the optimal stimulation parameters and/or subsets.

### DETAILED DESCRIPTION

While Spinal Cord Stimulation (SCS) therapy can be an effective means of alleviating a patient's pain, such stimulation can also cause paresthesia. Paresthesia—sometimes referred to a “supra-perception” therapy—is a sensation such as tingling, prickling, heat, cold, etc. that can accompany SCS therapy. Generally, the effects of paresthesia are mild, or at least are not overly concerning to a patient. Moreover, paresthesia is generally a reasonable tradeoff for a patient whose chronic pain has now been brought under control by SCS therapy. Some patients even find paresthesia comfortable and soothing.

Nonetheless, at least for some patients, SCS therapy would ideally provide complete pain relief without paresthesia—what is often referred to as “sub-perception” or sub-threshold therapy that a patient cannot feel. Effective sub-perception therapy may provide pain relief without paresthesia by issuing stimulation pulses at higher frequencies. Unfortunately, such higher-frequency stimulation may require more power, which tends to drain the battery 14 of the IPG 10. See, e.g., U.S. Patent Application Publication 2016/0367822. If an IPG's battery 14 is a primary cell and not rechargeable, high-frequency stimulation means that the IPG 10 will need to be replaced more quickly. Alternatively, if an IPG battery 14 is rechargeable, the IPG 10 will need to be charged more frequently, or for longer periods of time. Either way, the patient is inconvenienced.

In an SCS application, it is desirable to determine a stimulation program that will be effective for each patient. A significant part of determining an effective stimulation program is to determine a “sweet spot” for stimulation in each patient, i.e., to select which electrodes should be active (E) and with what polarities (P) and relative amplitudes (X %) to recruit and thus treat a neural site at which pain originates in a patient. Selecting electrodes proximate to this neural site of pain can be difficult to determine, and experimentation is typically undertaken to select the best combination of electrodes to provide a patient's therapy.

As described in U.S. Patent Application Publication 2019/0366104, which is hereby expressly incorporated by reference, selecting electrodes for a given patient can be even more difficult when sub-perception therapy is used, because the patient does not feel the stimulation, and therefore it can be difficult for the patient to feel whether the stimulation is “covering” his pain and therefore whether selected electrodes are effective. Further, sub-perception stimulation therapy may require a “wash in” period before it can become effective. A wash in period can take up to a day or more, and therefore sub-perception stimulation may not be immediately effective, making electrode selection more difficult.

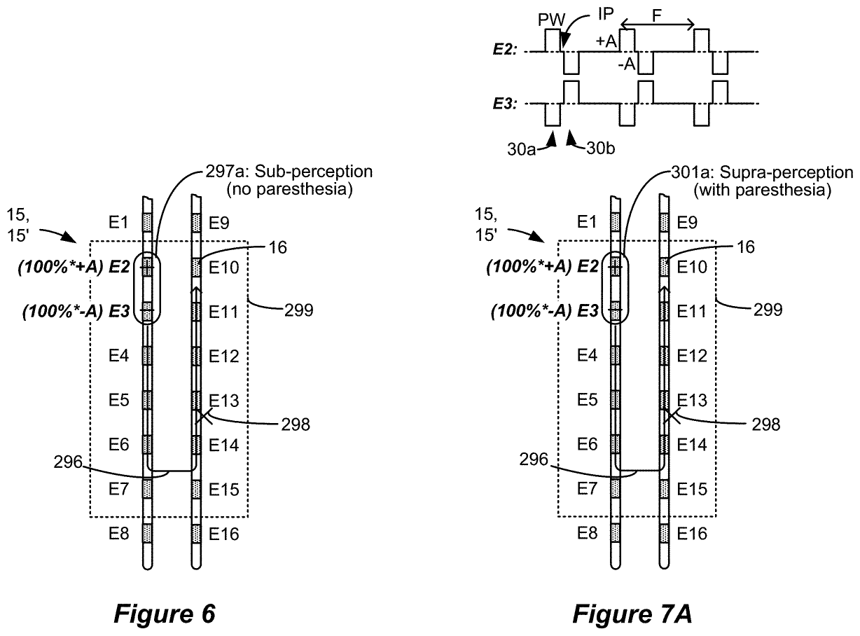

FIG. 6 briefly explains the '104 Publication's technique for a sweet spot search, i.e., how electrodes can be selected that are proximate to a neural site of pain 298 in a patient, when sub-perception stimulation is used. The technique of FIG. 6 is particularly useful in a trial setting after a patient is first implanted with an electrode array, i.e., after receiving their IPG or ETS.

In the example shown, it is assumed that a pain site 298 is likely within a tissue region 299. Such region 299 may be deduced by a clinician based on the patient symptoms, e.g., by understanding which electrodes are proximate to certain vertebrae (not shown), such as within the T9-T10 interspace. In the example shown, region 299 is bounded by electrodes E2, E7, E15, and E10, meaning that electrodes outside of this region (e.g., E1, E8, E9, E16) are unlikely to have an effect on the patient's symptoms. Therefore, these electrodes may not be selected during the sweet spot search depicted in FIG. 6, as explained further below.

In FIG. 6, a sub-perception bipole 297a is selected, in which one electrode (e.g., E2) is selected as an anode that will source a positive current (+A) to the patient's tissue, while another electrode (e.g., E3) is selected as a cathode that will sink a negative current (−A) from the tissue. This is similar to what was illustrated earlier with respect to FIG. 2, and biphasic stimulation pulses can be used employing active charge recovery. Because the bipole 297a provides sub-perception stimulation, the amplitude A used during the sweet spot search is titrated down until the patient no longer feels paresthesia. This sub-perception bipole 297a is provided to the patient for a duration, such as a few days, which allows the sub-perception bipole's potential effectiveness to “wash in,” and allows the patient to provide feedback concerning how well the bipole 297a is helping their symptoms. Such patient feedback can comprise a pain scale ranking. For example, the patient can rank their pain on a scale from 1-10 using a Numerical Rating Scale (NRS) or the Visual Analogue Scale (VAS), with 1 denoting no or little pain and 10 denoting a worst pain imaginable. As discussed in the '104 Publication, such pain scale ranking can be entered into the patient's external controller 45.

After the bipole 297a is tested at this first location, a different combination of electrodes is chosen (anode electrode E3, cathode electrode E4), which moves the location of the bipole 297 in the patient's tissue. Again, the amplitude of the current A may need to be titrated to an appropriate sub-perception level. In the example shown, the bipole 297a is moved down one electrode lead, and up the other, as shown by path 296 in the hope of finding a combination of electrodes that covers the pain site 298. In the example of FIG. 6, given the pain site 298's proximity to electrodes E13 and E14, it might be expected that a bipole 297a at those electrodes will provide the best relief for the patient, as reflected by the patient's pain score rankings. The particular stimulation parameters chosen when forming bipole 297a can be selected at the GUI 64 of the clinician programmer 50 or other external device (such as a patient external controller 45) and wirelessly telemetered to the patient's IPG or ETS for execution.

While the sweet spot search of FIG. 6 can be effective, it can also take a significantly long time when sub-perception stimulation is used. As noted, sub-perception stimulation is provided at each bipole 297 location for a number of days, and because a large number of bipole locations are chosen, the entire sweet spot search can take up to a month to complete.

The inventors have determined via testing of SCS patients that even if it is desired to eventually use sub-perception therapy for a patient going forward after the sweet spot search, it is beneficial to use supra-perception stimulation during the sweet spot search to select active electrodes for the patient. Use of supra-perception stimulation during the sweet spot search greatly accelerates determination of effective electrodes for the patient compared to the use of sub-perception stimulation, which requires a wash in period at each set of electrodes tested. After determining electrodes for use with the patient using supra-perception therapy, therapy may be titrated to sub-perception levels keeping the same electrodes determined for the patient during the sweet spot search. Because the selected electrodes are known to be recruiting the neural site of the patient's pain, the application of sub-perception therapy to those electrodes is more likely to have immediate effect, reducing or potentially eliminating the need to wash in the sub-perception therapy that follows. In short, effective sub-perception therapy can be achieved more quickly for the patient when supra-perception sweet spot searching is utilized. Preferably, supra-perception sweet spot searching occurs using symmetric biphasic pulses occurring at low frequencies—such as between 40 and 200 Hz in one example.

In accordance with one aspect of the disclosed technique, a patient will be provided sub-perception therapy. Sweet spot searching to determine electrodes that may be used during sub-perception therapy may precede such sub-perception therapy. In some aspects, when sub-perception therapy is used for the patient, sweet spot searching may use a bipole 297a that is sub-perception (FIG. 6), as just described. This may be relevant because the sub-perception sweet spot search may match the eventual sub-perception therapy the patient will receive.

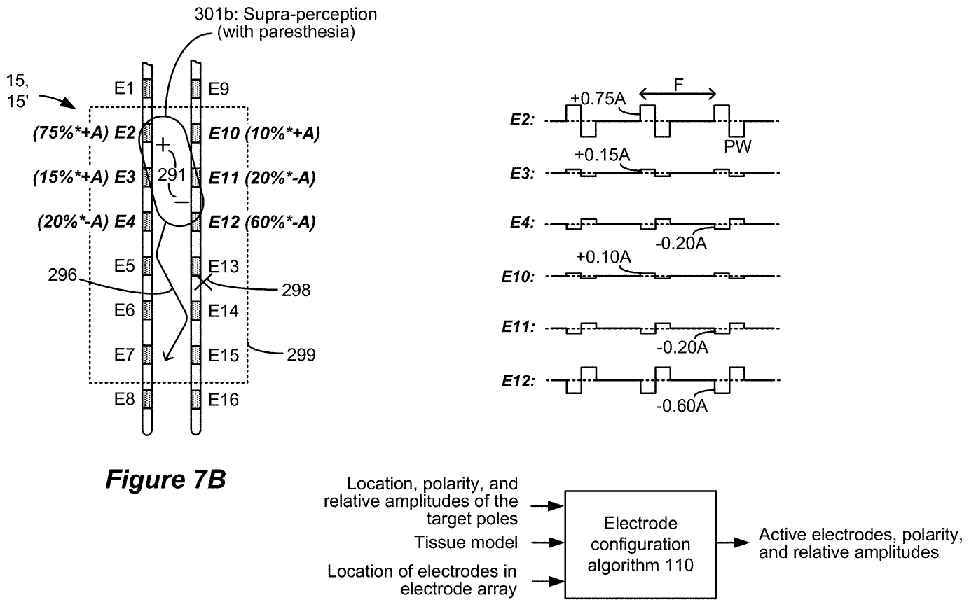

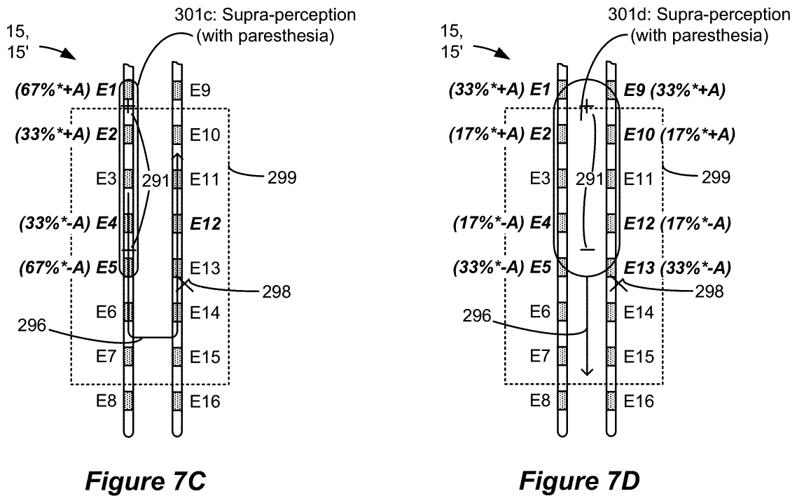

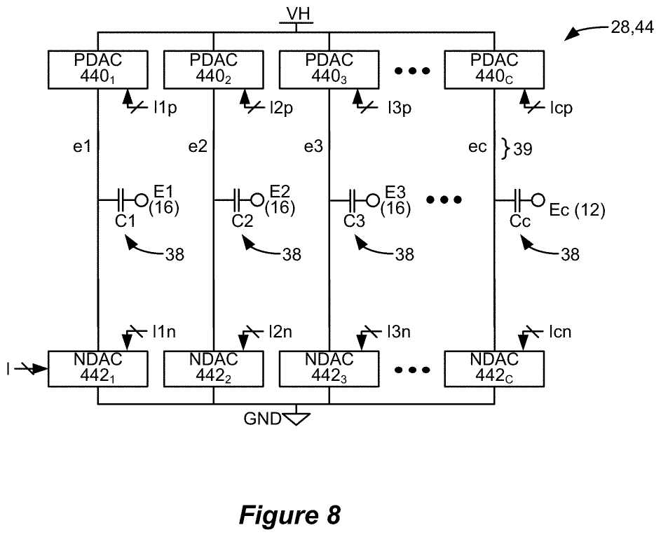

However, the inventors have determined that even if sub-perception therapy is eventually to be used for the patient, it can be beneficial to use supra-perception stimulation—that is, stimulation with accompanying paresthesia—during the sweet spot search. This is shown in FIG. 7A, where the movable bipole 301a provides supra-perception stimulation that can be felt by the patient. Providing bipole 301a as supra-perception stimulation can merely involve increasing its amplitude (e.g., current A) when compared to the sub-perception bipole 297a of FIG. 6, although other stimulation parameters might be adjusted as well, such as by providing longer pulse widths.

The inventors have determined that there are benefits to employing supra-perception stimulation during the sweet spot search even though sub-perception therapy will eventually be used for the patient.

First, as mentioned above, the use of supra-perception therapy by definition allows the patient to feel the stimulation, which enables the patient to provide essentially immediate feedback to the clinician whether the paresthesia seems to be well covering his pain site 298. In other words, it is not necessary to take the time to wash in bipole 301a at each location as it is moved along path 296. Thus, a suitable bipole 301a proximate to the patient's pain site 298 can be established much more quickly, such as within a single clinician's visit, rather than over a period of days or weeks. In one example, when sub-perception therapy is preceded with supra-perception sweet spot searching, the time needed to wash in the sub-perception therapy can be one hour or less, ten minutes or less, or even a matter of seconds. This allows wash in to occur during a single programming session during which the patient's IPG or ETS is programmed, and without the need for the patient to leave the clinician's office. Furthermore, the sub-perception therapy is noted to continue to be effective for some period of time after such therapy ceases, i.e., the therapy is effective during a “wash out” period after the cessation of therapy. The wash out period may be ten minutes or longer, or even an hour or longer. Note that this is beneficial because it means that therapy can be curtailed for some period of time (during the washout period), or rather that the sub-perception therapy can be cycled on and off. In short, the IPG does not need to continually provide the sub-perception therapy, and can effectively be off for periods of time, which saves power in the IPG.

Second, use of supra-perception stimulation during the sweet spot search ensures that electrodes are determined that well recruit the pain site 298. As a result, after the sweet spot search is complete and eventual sub-perception therapy is titrated for the patient, wash in of that sub-perception therapy may not take as long because the electrodes needed for good recruitment have already been confidently determined.

FIGS. 7B-7D show other supra-perception bipoles 301b-301d that may be used, and in particular show how the virtual bipoles may be formed using virtual poles by activating three or more of the electrodes 16. Virtual poles are discussed further in U.S. Patent Application Publication 2019/0175915, which is incorporated herein by reference in its entirety, and thus virtual poles are only briefly explained here. Forming virtual poles is assisted if the stimulation circuitry 28 or 44 used in the IPG or ETS is capable of independently setting the current at any of the electrodes—what is sometimes known as a Multiple Independent Current Control (MICC), which is explained further below with reference to FIG. 8.

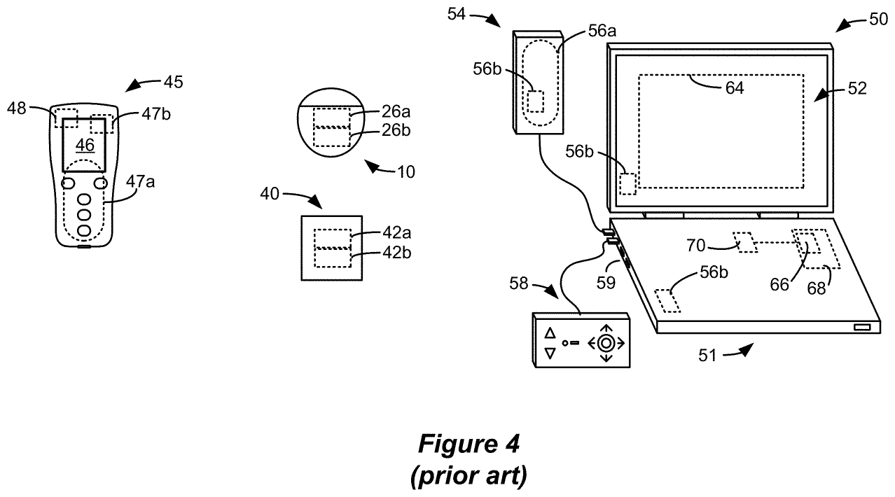

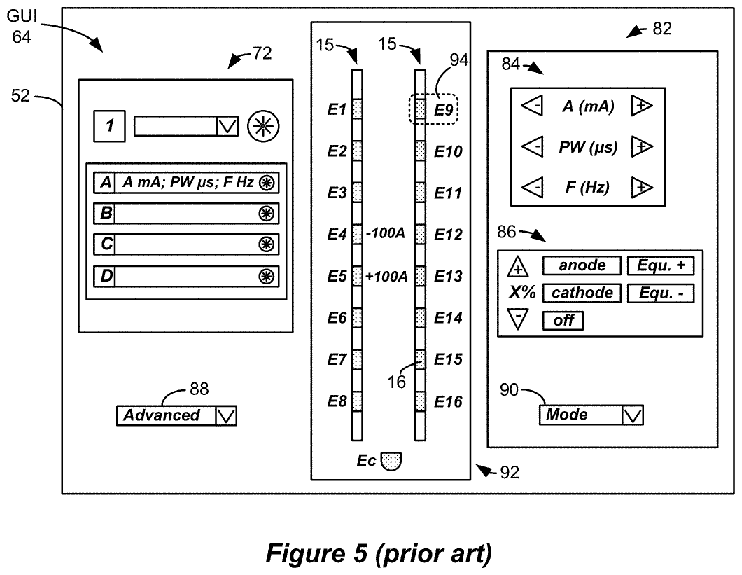

When a virtual bipole is used, the GUI 64 (FIG. 5) of the clinician programmer 50 (FIG. 4) can be used to define an anode pole (+) and a cathode pole (−) at positions 291 (FIG. 7B) that may not necessarily correspond to the position of the physical electrodes 16. The control circuitry 70 in the clinician programmer 50, executing an electrode configuration algorithm 110, can compute from these positions 291 and from other tissue modeling information which physical electrodes 16 will need to be selected and with what amplitudes to form the virtual anode and virtual cathode at the designated positions 291. As described earlier, amplitudes at selected electrodes may be expressed as a percentage X % of the total current amplitude A specified at the GUI 64 of the clinician programmer 50.

For example, in FIG. 7B, the virtual anode pole is located at a position 291 between electrodes E2, E3 and E10. The electrode configuration algorithm 110 operable in the clinician programmer 50 may then calculate based on this position that each of these electrodes (during first pulse phase 30a) will receive an appropriate share (X %) of the total anodic current +A to locate the virtual anode at this position. Since the virtual anode's position is closest to electrode E2, this electrode E2 may receive the largest share of the specified anodic current +A (e.g., 75%*+A). Electrodes E3 and E10 which are proximate to the virtual anode pole's position but farther away receive lesser shares of the anodic current (e.g., 15%*+A and 10%*+A respectively). Likewise, it can be seen that from the designated position 291 of the virtual cathode pole, which is proximate to electrodes E4, E11, and E12, that these electrodes will receive an appropriate share of the specified cathodic current −A (e.g., 20%*−A, 20%*−A, and 60%*−A respectively, again during the first pulse phase 30a). These polarities would then be flipped during the second phases 30b of the pulses, as shown in the waveforms of FIG. 7B. In any event, the use of virtual poles in the formation of bipole 301b allows the field in the tissue to be shaped, and many different combinations of electrodes can be tried during the sweet spot search. In this regard, it is not strictly necessary that the (virtual) bipole be moved along an orderly path 296 with respect to the electrodes, and the path may be randomized, perhaps as guided by feedback from the patient. Electrode configuration algorithm 110 is described further in U.S. Patent Application Publication 2019/0175915.

FIG. 7C shows a useful virtual bipole 301c configuration that can be used during the sweet spot search. This virtual bipole 301c again defines a target anode and cathode whose positions do not correspond to the position of the physical electrodes. The virtual bipole 301c is formed along a lead—essentially spanning the length of four electrodes from E1 to E5. This creates a larger field in the tissue better able to recruit the patient's pain site 298. This bipole configuration 301c may need to be moved to a smaller number of locations than would a smaller bipole configuration compared 301a of FIG. 7A) as it moves along path 296, thus accelerating pain site 298 detection. FIG. 7D expands upon the bipole configuration of FIG. 7C to create a virtual bipole 301d using electrodes formed on both leads, e.g., from electrodes E1 to E5 and from electrodes E9 to E13. This bipole 301d configuration need only be moved along a single path 296 that is parallel to the leads, as its field is large enough to recruit neural tissue proximate to both leads. This can further accelerate pain site detection.

In some aspects, the supra-perception bipoles 301a-301d used during the sweet spot search comprise symmetric biphasic waveforms having actively-driven (e.g., by the stimulation circuitry 28 or 44) pulse phases 30a and 30b of the same pulse width PW and the same amplitude (with the polarity flipped during the phases) (e.g., A₃₀ₐ=A_{30b}, and PW₃₀ₐ=PW_{30b}). This is beneficial because the second pulse phase 30b provides active charge recovery, with in this case the charge provided during the first pulse phase 30a (Q₃₀ₐ) equaling the charge of the second pulse phase 30b (Q_{30b}), such that the pulses are charge balanced. Use of biphasic waveforms are also believed beneficial because, as is known, the cathode is largely involved in neural tissue recruitment. When a biphasic pulse is used, the positions of the (virtual) anode and cathode will flip during the pulse's two phases. This effectively doubles the neural tissue that is recruited for stimulation, and thus increases the possibility that the pain site 298 will be covered by a bipole at the correct location.

The supra-perception bipoles 301a-301d do not however need to comprise symmetric biphasic pulses as just described. For example, the amplitude and pulse width of the two phases 30a and 30b can be different, while keeping the charge (Q) of the two phases balanced (e.g., Q₃₀ₐ=A₃₀ₐ*PW₃₀ₐ=A_{30b}*PW_{30b}=Q_{30b}). Alternatively, the two phases 30a and 30b may be charge imbalanced (e.g., Q₃₀ₐ=A₃₀ₐ*PW₃₀ₐ>A_{30b}*PW_{30b}=Q_{30b}, or Q₃₀ₐ=A₃₀ₐ*PW₃₀ₐ<A_{30b}*PW_{30b}=Q_{30b}). In short, the pulses in bipoles 301-301d can be biphasic symmetric (and thus inherently charge balanced), biphasic asymmetric but still charge balanced, or biphasic asymmetric and charge imbalanced.

In a preferred example, the frequency F of the supra-perception pulses 301a-301d used during the supra-perception sweet spot search may be 10 kHz or less, 1 kHz or less, 500 Hz or less, 300 Hz or less, 200 Hz or less, 130 Hz or less, or 100 Hz or less, or ranges bounded by two of these frequencies (e.g., 100-130 Hz, or 100-200 Hz). In particular examples, frequencies of 90 Hz, 40 Hz, or 10 Hz can be used, with pulses comprising biphasic pulses which are preferably symmetric. However, a single actively-driven pulse phase followed by a passive recovery phase could also be used. The pulse width PW may also comprise a value in the range of hundreds of microseconds, such as 150 to 400 microseconds. Because the goal of supra-perception sweet spot searching is merely to determine electrodes that appropriately cover a patient's pain, frequency and pulse width may be of less importance at this stage. Once electrodes have been chosen for sub-perception stimulation, frequency and pulse width can be optimized, as discussed further below.

It should be understood that the supra-perception bipoles 301a-301d used during sweet spot searching need not necessarily be the same electrodes that are selected when later providing the patient with sub-perception therapy. Instead, the best location of the bipole noticed during the search can be used as the basis to modify the selected electrodes. Suppose for example that a bipole 301a (FIG. 7A) is used during sweep spot searching, and it is determined that bipole provides the best pain relief when located at electrodes E13 and E14. At that point, sub-perception therapy using those electrodes E13 and E14 can be tried for the patient going forward. Alternatively, it may be sensible to modify the selected electrodes to see if the patient's symptoms can be further improved before sub-perception therapy is tried. For example, the distance (focus) between the cathode and anode can be varied, using virtual poles as already described. Or, a tripole (anode/cathode/anode) consisting of electrodes E12/E13/E14 or E13/E14/E15 could be tried. See U.S. Patent Application Publication 2019/0175915 (discussing tripoles). Or electrodes on a different lead could also be tried in combination with E13 and E14. For example, because electrodes E5 and E6 are generally proximate to electrodes E13 and E14, it may be useful to add E5 or E6 as sources of anodic or cathodic current (again creating virtual poles). All of these types of adjustments should be understood as comprising “steering” or an adjustment to the “location” at which therapy is applied, even if a central point of stimulation doesn't change (as can occur for example when the distance or focus between the cathode and anode is varied).

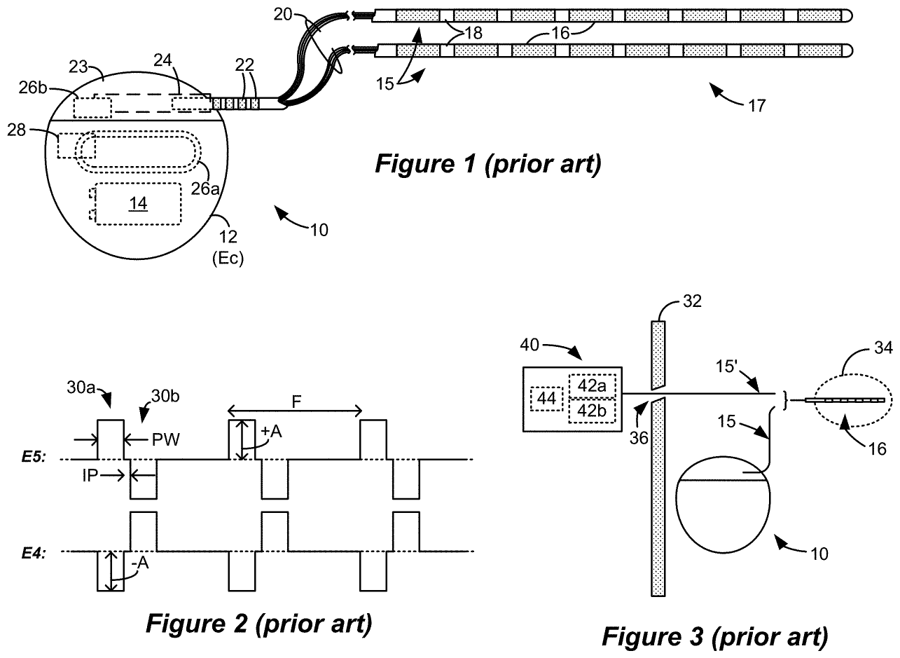

Multiple Independent Current Control (MICC) is explained in one example with reference to FIG. 8, which shows the stimulation circuitry 28 (FIG. 1) or 44 (FIG. 3) in the IPG or ETS used to form prescribed stimulation at a patient's tissue. The stimulation circuitry 28 or 44 can control the current or charge at each electrode independently, and using GUI 64 (FIG. 5) allows the current or charge to be steered to different electrodes, which is useful for example when moving the bipole 301i along path 296 during the sweet spot search (FIG. 7A-7D). The stimulation circuitry 28 or 44 includes one or more current sources 440ᵢ and one or more current sinks 442ᵢ. The sources and sinks 440ᵢ and 442ᵢ can comprise Digital-to-Analog converters (DACs), and may be referred to as PDACs 440ᵢ and NDACs 442ᵢ in accordance with the Positive (sourced, anodic) and Negative (sunk, cathodic) currents they respectively issue. In the example shown, a NDAC/PDAC 440ᵢ/442ᵢ pair is dedicated (hardwired) to a particular electrode node ei 39. Each electrode node ei 39 is preferably connected to an electrode Ei 16 via a DC-blocking capacitor Ci 38, which act as a safety measure to prevent DC current injection into the patient, as could occur for example if there is a circuit fault in the stimulation circuitry 28 or 44. PDACs 440ᵢ and NDACs 442ᵢ can also comprise voltage sources.

Proper control of the PDACs 440ᵢ and NDACs 442ᵢ via GUI 64 allows any of the electrodes 16 and the case electrode Ec 12 to act as anodes or cathodes to create a current through a patient's tissue. Such control preferably comes in the form of digital signals Tip and Iin that set the anodic and cathodic current at each electrode Ei. If for example it is desired to set electrode E1 as an anode with a current of +3 mA, and to set electrodes E2 and E3 as cathodes with a current of −1.5 mA each, control signal I1p would be set to the digital equivalent of 3 mA to cause PDAC 440₁ to produce+3 mA, and control signals I2n and I3n would be set to the digital equivalent of 1.5 mA to cause NDACs 442₂ and 442₃ to each produce −1.5 mA. Note that definition of these control signals can also occur using the programmed amplitude A and percentage X % set in the GUI 64. For example, A may be set to 3 mA, with E1 designated as an anode with X=100%, and with E2 and E3 designated at cathodes with X=50%. Alternatively, the control signals may not be set with a percentage, and instead the GUI 64 can simply prescribe the current that will appear at each electrode at any point in time.

In short, the GUI 64 may be used to independently set the current at each electrode, or to steer the current between different electrodes. This is particularly useful in forming virtual bipoles, which as explained earlier involve activation of more than two electrodes. MICC also allows more sophisticated electric fields to be formed in the patient's tissue.

Other stimulation circuitries 28 can also be used to implement MICC. In an example not shown, a switching matrix can intervene between the one or more PDACs 440ᵢ and the electrode nodes ei 39, and between the one or more NDACs 442ᵢ and the electrode nodes. Switching matrices allows one or more of the PDACs or one or more of the NDACs to be connected to one or more electrode nodes at a given time. Various examples of stimulation circuitries can be found in U.S. Pat. Nos. 6,181,969, 8,606,362, 8,620,436, and U.S. Patent Application Publications 2018/0071513, 2018/0071520, and 2019/0083796.

Much of the stimulation circuitry 28 or 44, including the PDACs 440ᵢ and NDACs 442ᵢ, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with the IPG's or ETS's telemetry antennas), circuitry for generating the compliance voltage VH that powers the stimulation circuitry, various measurement circuits, etc.

While it is preferred to use sweet spot searching, and in particular supra-perception sweet spot searching, to determine the electrodes to be used during subsequent sub-perception therapy, it should be noted that this is not strictly necessary. Sub-perception therapy can be preceded by sub-perception sweet spot searching, or may not be preceded by sweet spot searching at all. In short, sub-perception therapy as described next is not reliant on the use of any sweet spot search.

In another aspect of the invention, the inventors have determined via testing of SCS patients that statistically significant correlations exists between pulse width (PW) and frequency (F) where an SCS patient will experience a reduction in back pain without paresthesia (sub-perception). Use of this information can be helpful in deciding what pulse width is likely optimal for a given SCS patient based on a particular frequency, and in deciding what frequency is likely optimal for a given SCS patient based on a particular pulse width. Beneficially, this information suggests that paresthesia-free sub-perception SCS stimulation can occur at frequencies of 10 kHz and below. Use of such low frequencies allows sub-perception therapy to be used with much lower power consumption in the patient's IPG or ETS.

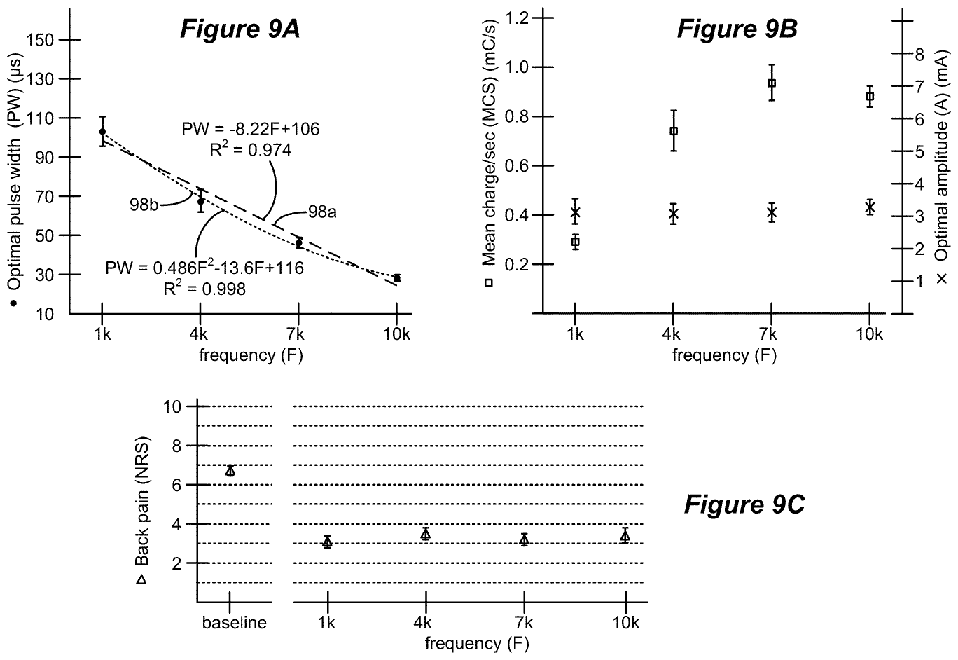

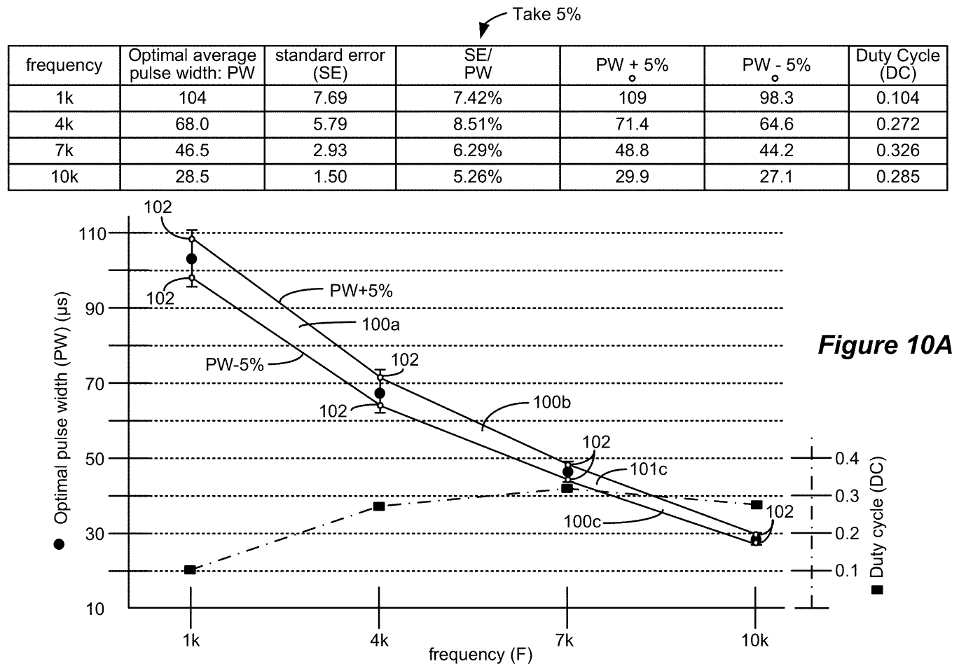

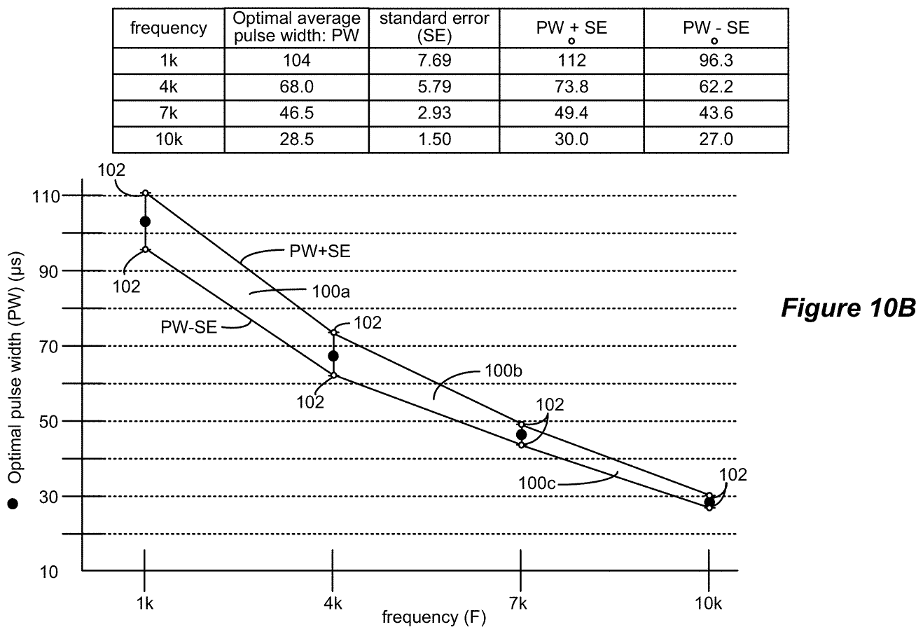

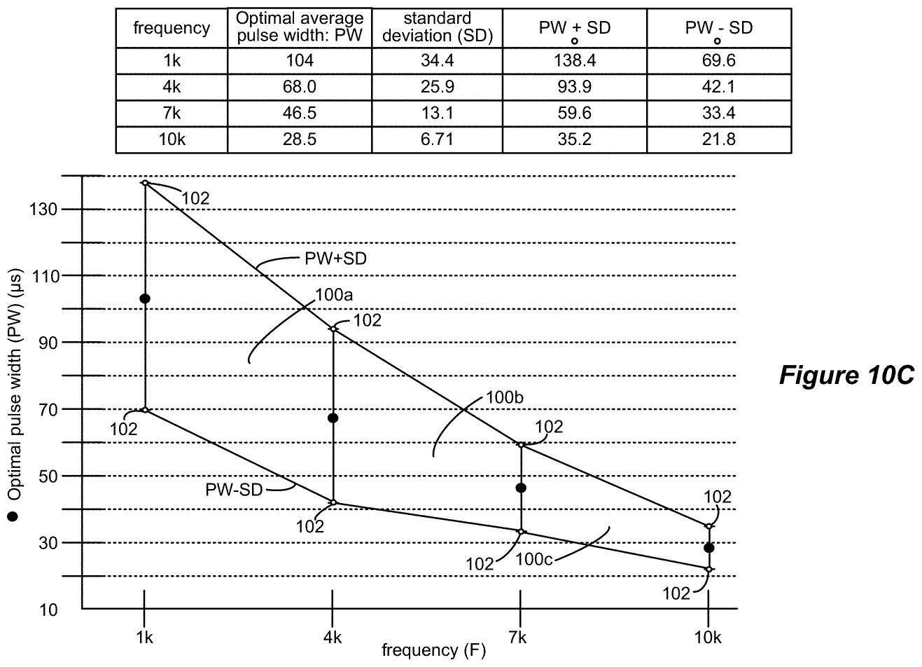

FIGS. 9A-10C shows results derived from testing patients at frequencies within a range of 1 kHz to 10 kHz. Details concerning how data was gathered from actual SCS patients, and the criteria for patient inclusion in the study, is explained in PCT (Int'l) Patent Application Publication WO 2021/178105 (the '105 Publication), which is incorporated by reference in its entirety. FIGS. 9A-9C show the results of testing the patients at 10 kHz, 7 kHz, 4 Hz and 1 kHz. Data is shown in each figure as average values for the 20 remaining patients at each frequency, with error bars reflecting standard error (SE) between the patients.

Starting with FIG. 9B, the optimized amplitude A for the 20 remaining patients are shown at the tested frequencies. Interestingly, the optimal amplitude at each frequency was essentially constant—around 3 mA. FIG. 9B also shows the amount of energy expended at each frequency, more specifically a mean charge per second (MCS) (in mC/s) attributable to the pulses. MCS is computed by taking the optimal pulse width (FIG. 9A, discussed next) and multiplying it by the optimal amplitude (A) and the frequency (F), which MCS value can comprise a neural dose. MCS correlates to the current or power that the battery in the IPG 10 must expend to form the optimal pulses. Significantly, the MCS is significantly lower at lower frequencies: for example, the MCS at F=1 kHz is approximately ⅓ of its value at higher frequencies (e.g., F=7 kHz or 10 kHz). This means that optimal SCS therapy—that alleviates back pain without paresthesia—is achievable at lower frequencies like F=1 kHz, with the added benefit of lower power draws that are more considerate of the IPG 10's (or ETS 40's) battery.

FIG. 9A shows optimal pulse width as a function of frequency for the 1 kHz to 10 kHz frequency range tested. As shown, the relationship follows a statistically significant trend: when modeled using linear regression 98a, PW=−8.22F+106, where pulse width is measured in microseconds and frequency is measured in kiloHertz, with a correlation coefficient R² of 0.974; when modeled using polynomial regression 98b, PW=0.486F²−13.6F+116, again with pulse width measured in microseconds and frequency measured in kiloHertz, with an even better correlation coefficient of R²=0.998. Other fitting methods could be used to establish other information relating frequency and pulse width at which stimulation pulses are formed to provide pain relief without paresthesia in the frequency range of 1 kHz to 10 kHz.

Note that the relationship between optimal pulse width and frequency is not simply an expected relationship between frequency and duty cycle (DC), i.e., the duration that a pulse is ‘on’ divided by its period (1/F). In this regard, notice that a given frequency has a natural effect on pulse width: one would expect that a higher frequency pulses would have smaller pulse widths. Thus, it might be expected for example that a 1 kHz waveform with a 100 microsecond pulse width would have the same clinical results as a 10 kHz waveform with a 10 microsecond frequency, because the duty cycle of both of these waveforms is 10%. FIG. 10A shows the resulting duty cycle of the stimulation waveforms using the optimal pulse width in the frequency range of 1 kHz to 10 kHz. Here, duty cycle is computed by considering the total ‘on’ time of the first pulse phase 30a (FIG. 2) only; the duration of the symmetric second pulse phase is ignored. This duty cycle is not constant over the 1 kHz to 10 kHz frequency range: for example, the optimal pulse width at 1 kHz (104 microseconds) is not merely ten times the optimal pulse width at 10 kHz (28.5 microseconds). Thus, there is significance to the optimal pulse widths beyond a mere scaling of the frequency.

FIG. 9C shows average patient pain scores at the optimal stimulation parameters (optimal amplitude (FIG. 7B) and pulse width (FIG. 7A) for each frequency in the range of 1 kHz to 10 kHz. As noted earlier, patients in the study, prior to receiving SCS therapy, initially reported pain scores with an average of 6.75. After SCS implantation and during the study, and with amplitude and pulse width optimized during the provisional of sub-perception therapy, their average pain scores dropped significantly, to an average score of about 3 for all frequencies tested.

FIG. 10A provides a deeper analysis of the resulting relationship between optimal pulse width and frequency in the frequency range of 1 kHz to 10 kHz. The chart in FIG. 10A shows the average optimal pulse width for the 20 patients in the study at each frequency, along with the standard error resulting from variations between them. These are normalized at each frequency by dividing the standard error by the optimal pulse width, ranging in variations at each frequency between 5.26% and 8.51%. From this, a 5% variance (lower than all computed values) can be assumed as a statistically-significant variance at all frequencies tested.

From this 5% variance, a maximum average pulse width (PW+5%) and a minimum average pulse width (PW+5%) can be calculated for each frequency. For example, the optimal average pulse width PW at 1 kHz is 104 microseconds, and 5% above this value (1.05*104 μs) is 109 μs; 5% below this value (0.95*104) is 98.3 μs. Likewise, the optimal average pulse width AVG(PW) at 4 kHz is 68.0 microseconds, and 5% above this value (1.05*68.0 μs) is 71.4 μs; 5% below this value (0.95*68.0 μs) is 64.6 μs. Thus, a statistically-significant reduction in pain without paresthesia occurs in or on the linearly bounded region 100a of points 102 of (1 kHz, 98.3 μs), (1 kHz, 109 μs), (4 kHz, 71.4 μs), and (4 kHz, 64.6 μs). A linearly bounded region 100b around points 102 is also defined for frequencies greater than or equal to 4 kHz and less than or equal to 7 kHz: (4 kHz, 71.4 μs), (4 kHz, 64.6 μs), (7 kHz, 44.2 μs), (7 kHz, 48.8 μs). A linear bounded region 100c around points 102 is also defined for frequencies greater than or equal to 7 kHz and less than or equal to 10 kHz: (7 kHz, 44.2 μs), (7 kHz, 48.8 μs), (10 kHz, 29.9 μs), (10 kHz, 27.1 μs). Such regions 100 thus comprise information relating frequency and pulse width at which stimulation pulses are formed to provide pain relief without paresthesia in the frequency range of 1 kHz to 10 kHz.

FIG. 10B provides an alternative analysis of the resulting relationship between optimal pulse width and frequency. In this example, regions 100a-100c are defined based upon the standard error (SE) calculated at each frequency. Thus, points 102 defining the corners of the regions 100a-c are simply located at the extent of the SE error bars at each frequency (PW+SE, and PW− SE), even though these error bars are of different magnitudes at each frequency. Thus, a statistically-significant reduction in pain without paresthesia occurs in or on the linearly bounded region 100a of points (1 kHz, 96.3 μs), (1 kHz, 112 μs), (4 kHz, 73.8 μs), and (4 kHz, 62.2 μs). The linear bounded regions 100b and 100c are similar, and because the points 102 defining them are set forth in chart at the top of FIG. 10B, they are not repeated here.

FIG. 10C provides another analysis of the resulting relationship between optimal pulse width and frequency. In this example, regions 100a-100c are defined based upon the standard deviation (SD) calculated at each frequency, which is larger than the standard error (SE) metric used to this point. Points 102 defining the corners of the regions 100a-c are located at the extent of the SD error bars at each frequency (PW+SD, and PW− SD), although points 102 could also be set within the error bars, similar to what was illustrated earlier with respect to FIG. 10A. In any event, a statistically-significant reduction in pain without paresthesia occurs in or on the linearly bounded region 100a of points (1 kHz, 69.6 μs), (1 kHz, 138.4 μs), (4 kHz, 93.9 μs), and (4 kHz, 42.1 μs). The linear bounded regions 100b and 100c are similar, and because the points 102 defining them are set forth in chart at the top of FIG. 10C, they are not repeated here.

More generally, although not illustrated, regions within the frequency range of 1 kHz to 10 kHz where sub-perception efficacy was achieved comprises linearly-bounded region 100a (1 kHz, 50.0 μs), (1 kHz, 200.0 μs), (4 kHz, 110.0 μs), and (4 kHz, 30.0 μs); and/or linearly-bounded region 100b (4 kHz, 110.0 μs), (4 kHz, 30.0 μs), (7 kHz, 30.0 μs), and (7 kHz, 60.0 μs); and/or linearly-bounded region 100c (7 kHz, 30.0 μs), (7 kHz, 60.0 μs), (10 kHz, 40.0 μs), and (10 kHz, 20.0 μs).

In summary, one or more statistically-significant regions 100 can be defined for the optimal pulse width and frequency data taken for the patients in the study to arrive at combinations of pulse width and frequency that reduce pain without the side effect of paresthesia within the frequency range of 1 kHz to 10 kHz, and different statistical measures of error can be used to so define the one or more regions.

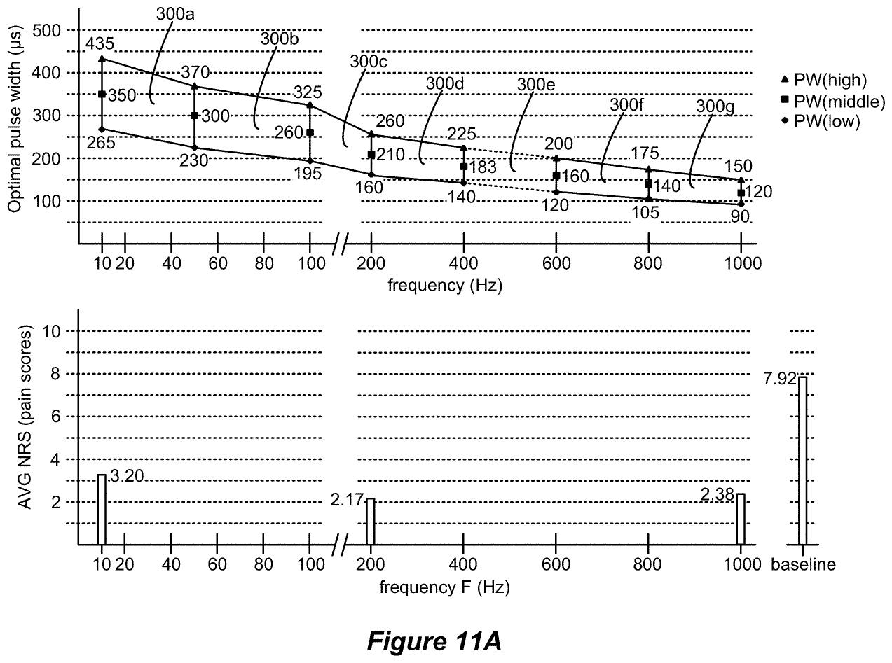

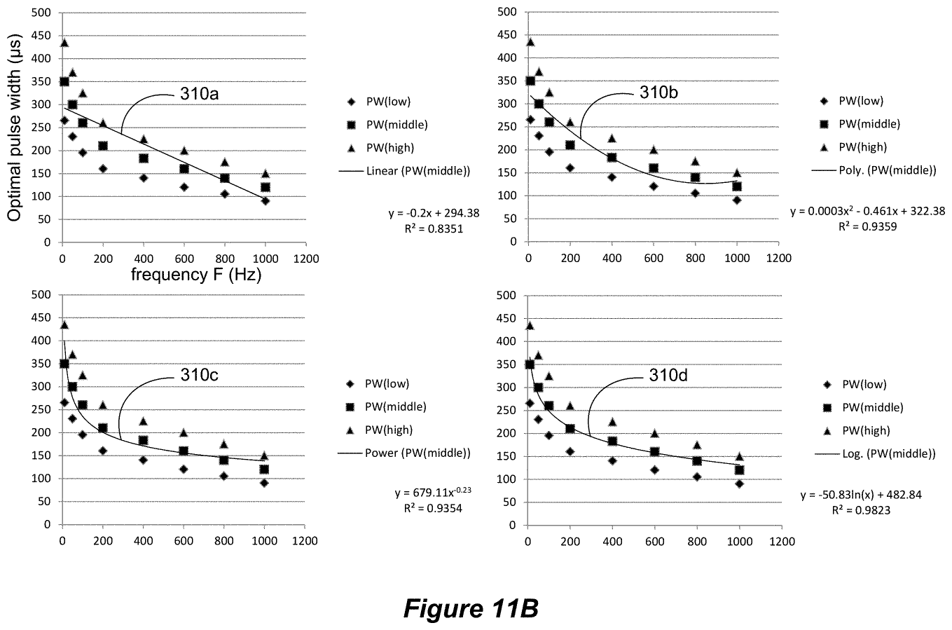

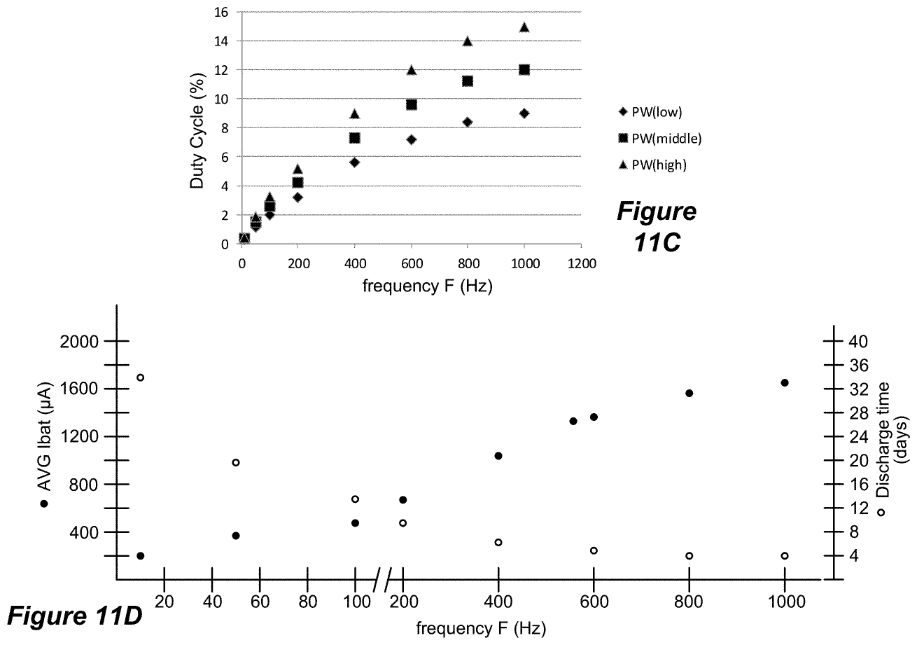

FIGS. 11A-11D show the results of testing other patients with sub-perception stimulation therapy at frequencies at or below 1 kHz. Testing of the patients generally occurred after supra-perception sweep spot searching occurred to select appropriate electrodes (E), polarities (P) and relative amplitudes (X %) for each patient (see FIGS. 7A-7D), although again the sub-perception electrodes used could vary from those used during the supra-perception sweet spot search (e.g., using MICC). Patients were tested with sub-perception stimulation using symmetric biphasic bipoles, although the form of pulses used during sub-perception therapy could vary.

FIG. 11A shows the relationship between frequency and pulse width at which effective sub-perception therapy was reported by patients for frequencies of 1 kHz and below. As can be seen, at each frequency tested, the optimal pulse width again fell within a range. For example, at 800 Hz, patients reported good results when the pulse width fell within a range of 105-175 microseconds. The upper end of the pulse width range at each frequency is denoted PW(high), while the lower end of the pulse width range at each frequency is denoted PW(low). PW(middle) denotes the middle (e.g., average) of the PW(high) and PW(low) at each frequency. At each of the tested frequencies the amplitude of the current provided (A) was titrated down to sub-perception levels, such that the patient could not feel paresthesia. Typically, the current was titrated to 80% of the threshold at which paresthesia could be sensed. Because each patient's anatomy is unique, the sub-perception amplitude A could vary from patient to patient. The pulse width data depicted comprises the pulse width of only the first phase of the stimulation pulses.

Table 1 below expresses the optimal pulse width versus frequency data of FIG. 11A in tabular form for frequencies at or below 1 kHz, with the pulse widths expressed in microseconds:

TABLE 1

| Frequency | PW(low) | PW(middle) | PW(high) |
|---|---|---|---|
| (Hz) | (μs) | (μs) | (μs) |
| 1000 | 90 | 120 | 150 |
| 800 | 105 | 140 | 175 |
| 600 | 120 | 160 | 200 |
| 400 | 140 | 183 | 225 |
| 200 | 160 | 210 | 260 |
| 100 | 195 | 260 | 325 |
| 50 | 230 | 300 | 370 |
| 10 | 265 | 350 | 435 |

As with the analysis described earlier for frequencies in a range of 1 kHz to 10 kHz (FIGS. 9A-10C), the data may be broken down to define different regions 300i at which effective sub-perception therapy is realized below 1 kHz. For example, regions of effective sub-perception therapy may be linearly bounded between various frequencies and the high and low pulse widths that define effectiveness. For example, at 10 Hz, PW(low)=265 microseconds and PW(high)=435 microseconds. At 50 Hz, PW(low)=230 microseconds and PW(high)=370 microseconds. Therefore, a region 300a that provides good sub-perception therapy is defined by the linearly bounded region of points (10 Hz, 265 μs), (10 Hz, 435 μs), (50 Hz, 370 μs), and (50 Hz, 230 μs). Table 2 defines the points that linearly bind each of the regions 300a-300g shown in FIG. 11A:

TABLE 2

| region | Bounded by points (Hz, μs) |
|---|---|
| 300a | (10, 265), (10, 435), (50, 370), (50, 230) |
| 300b | (50, 230), (50, 370), (100, 325), (100, 195) |
| 300c | (100, 195), (100, 325), (200, 260), (200, 160) |
| 300d | (200, 160), (200, 260), (400, 225), (400, 140) |
| 300e | (400, 140), (400, 225), (600, 200), (600, 120) |
| 300f | (600, 120), (600, 200), (800, 175), (800, 105) |
| 300g | (800, 105), (800, 175), (1000, 150), (1000, 90) |

Regions of sub-perception therapeutic effectiveness at frequencies at or below 1 kHz may be defined in other statistically-significant ways, such as those described earlier for frequencies in the range of 1 kHz to 10 kHz (FIGS. 10A-10C). For example, regions 300i may be defined by reference to the pulse width at the middle of the ranges at each frequency, PW(middle). PW(middle) may comprise for example an average optimal pulse width reported by patients at each frequency, rather than as a strict middle of an effective range reported by those patients. PW(high) and PW(low) may then be determined as a statistical variance from the average PW(middle) at each frequency, and used to set the upper and lower bounds of effective sub-perception regions. For example, PW(high) may comprise average PW(middle) plus a standard deviation or standard error, or a multiples of such statistical measures; PW(low) may likewise comprise average PW(middle) minus a standard deviation or standard error, or a multiple of such statistical measures. PW(high) and PW(low) may also be determined from average PW(middle) in other ways. For example, PW(high) may comprise average PW(middle) plus a set percentage, while PW(low) may comprise PW(middle) minus a set percentage. In summary, one or more statistically-significant regions 300 can be defined for the optimal pulse width and frequency data at frequencies at or below 1 kHz that reduce pain using sub-perception stimulation without the side effect of paresthesia.

Also shown in FIG. 11A are average patient pain scores (NRS scores) reported by patients when optimal pulse widths are used for different frequencies at 1 kHz or below. Prior to receiving SCS therapy, patients initially reported pain scores with an average of 7.92. After SCS implantation, and using the sub-perception stimulation at optimal pulse widths with the ranges shown at each frequency, the patients' average pain scores dropped significantly. At 1 kHz, 200 Hz, and 10 Hz, patients reported average pain scores of 2.38, 2.17, and 3.20 respectively. Thus clinical significance with respect to pain relief is shown when the optimal pulse widths are used at or below 1 kHz with sub-perception therapy.

The optimal pulse width versus frequency data of FIG. 11A for frequencies at or below 1 kHz is analyzed in FIG. 11B from the perspective of the middle pulse width, PW(middle) at each frequency (F). As shown, the relationships 310a-310d follows statistically significant trends, as evidenced by the various regression models shown in FIG. 11B and summarized in Table 3 below:

TABLE 3

| | | Correlation |
|---|---|---|
| Regression | Relationship | coefficient |
| model | (PW(middle) in μs) | R² |
| Linear | PW(middle) = −0.2F + 294.4 | 0.835 |
| (310a) | | |
| Polynomial | PW(middle) = 0.0002F² − | 0.936 |
| (310b) | 0.461F + 332.38 | |
| Power | PW(middle) = 679.1×^{-0.23} | 0.935 |
| (310c) | | |
| Logarithmic | PW(middle) = −50.83ln(F) + | 0.982 |
| (310d) | 482.8 | |

Other fitting methods could be used to establish other information relating frequency and pulse width at which stimulation pulses are formed to provide sub-perception pain relief without paresthesia.

Regression analysis can also be used to define statistically relevant regions such as 300a-300g where sub-perception therapy is effective at or below 1 kHz. For example, and although not shown in FIG. 11B, regression can be performed for PW(low) v. F to set a lower boundary of relevant regions 300i, and regression can be performed for PW(high) v. F to set an upper boundary of relevant regions 300i.

Note that the relationship between optimal pulse width and frequency depicted in FIG. 11A is not simply an expected relationship between frequency and duty cycle (DC), as FIG. 11C shows. As was the case when the 1 kHz to 10 kHz frequency range was tested (FIG. 10A), the duty cycle of the optimal pulse widths is not constant at 1 kHz and below. Again, there is significance to the optimal pulse widths beyond a mere scaling of the frequency. Nonetheless, most of the pulse widths observed to be optimal at 1 kHz and below are greater than 100 microseconds. Such pulse widths are not even possible at higher frequencies. For example, at 10 kHz, both pulse phases have to fit within a 100 us period, so PW longer than 100 are not even possible.

FIG. 11D shows further benefits achieved in using sub-perception at frequencies of 1 kHz and below, namely reduced power consumption. Two sets of data are graphed. The first data set comprises the average current drawn by the battery in the patients' IPG or ETS (AVG Ibat) at each frequency using the optimal pulse width for that patient (FIG. 11A) and the current amplitude A necessary to achieve sub-perception stimulation for that patient (again, this amplitude can vary for each of the patients). At 1 kHz, this average battery current is about 1700 microamps. However, as the frequency is reduced, this average battery current drops, to about 200 microamps at 10 Hz. The second data set looks at power consumption from a different vantage point, namely the number of days that an IPG or ETS with a fully-charged rechargeable battery can operate before recharge is required (“discharge time”). As would be expected based on the average battery current data, the discharge time is lower at higher frequencies when the average battery current is higher (e.g., about 3.9 days at 1 kHz, depending on various charging parameters and settings), and is higher at lower frequencies when the average battery current is lower (e.g., about 34 days at 10 Hz, depending on various charging parameters and settings). This is significant: not only can effective sub-perception therapy be provided at 1 kHz and below when optimal pulse widths are used; power consumptions is greatly lowered, which places less stress on the IPG or ETS, and allows it to operate from longer periods of time. As noted above, excessive power consumption is a significant problem when sub-perception therapy is traditionally used at higher frequencies. Note that the data of FIG. 11D could also be analyzed in terms of mean charge-per-second (MCS), as described earlier for the 1 kHz to 10 kHz data (FIG. 9B).

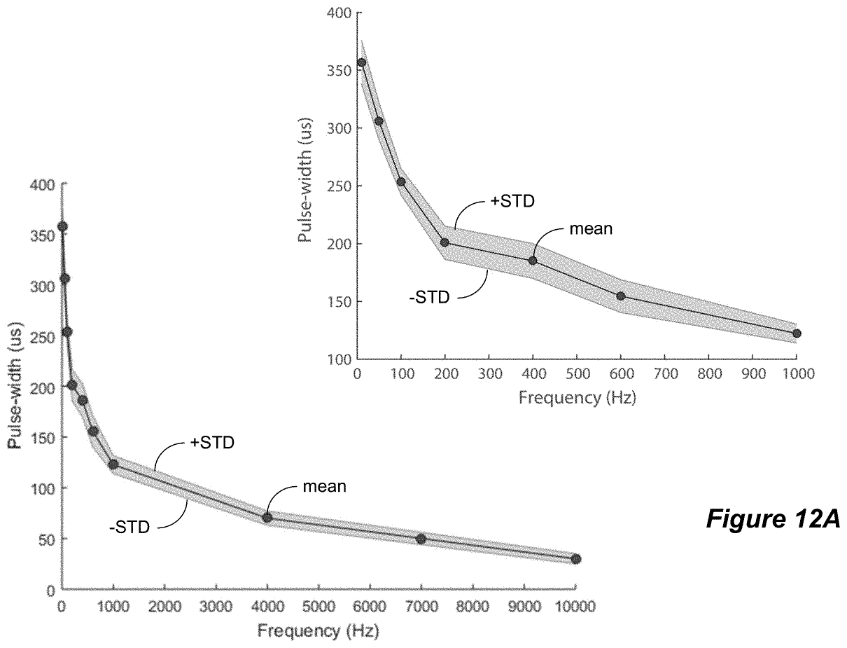

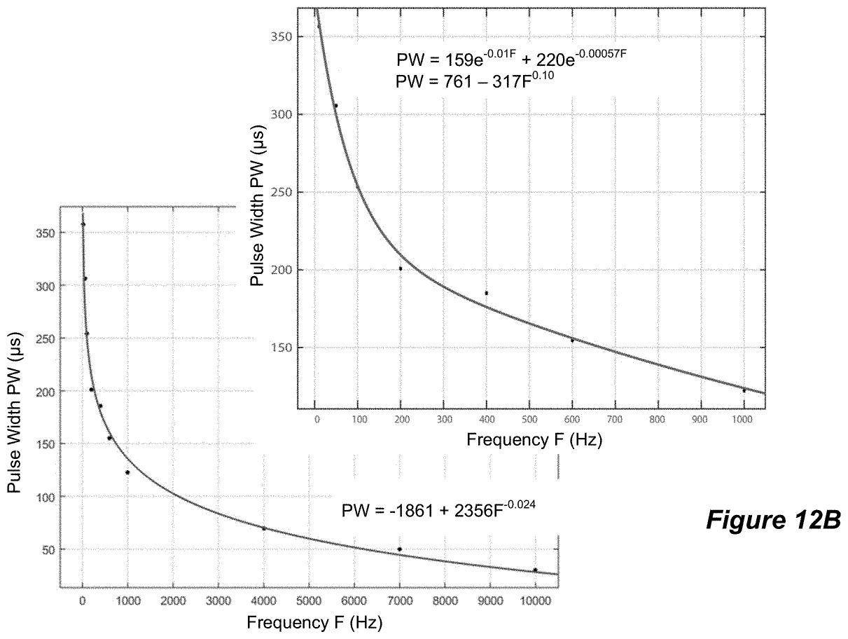

FIGS. 12A and 12B shows the results of additional testing that verifies the frequency versus pulse width relationships just presented. Here, data is shown for 25 patients tested using sub-perception stimulation at frequencies of 10 kHz and below. FIG. 12A shows two different graphs showing the result for frequencies of 10 k and below(lower graph) and for frequencies of 1 kHz and below (upper graph). Mean values are shown frequencies and pulse width values at which optimal sub-perception therapy is produced. Upper and lower bands denote one standard deviation's variance (+STD and −STD) above and below the mean. FIG. 12B shows curve fitting results as determined using mean values. Data for 1 kHz and below is fit with an exponential function and with a power function, resulting in relationship PW=159e^{-0.01F}+220e^{-0.00057F} and PW=761-317F^{0.10}, both of which well fit to the data. Data for 10 kHz and below is fit with a power function, yielding PW=−1861+2356F^{-0.024}, again with a good fit. The data could bit fit to other mathematical functions as well.

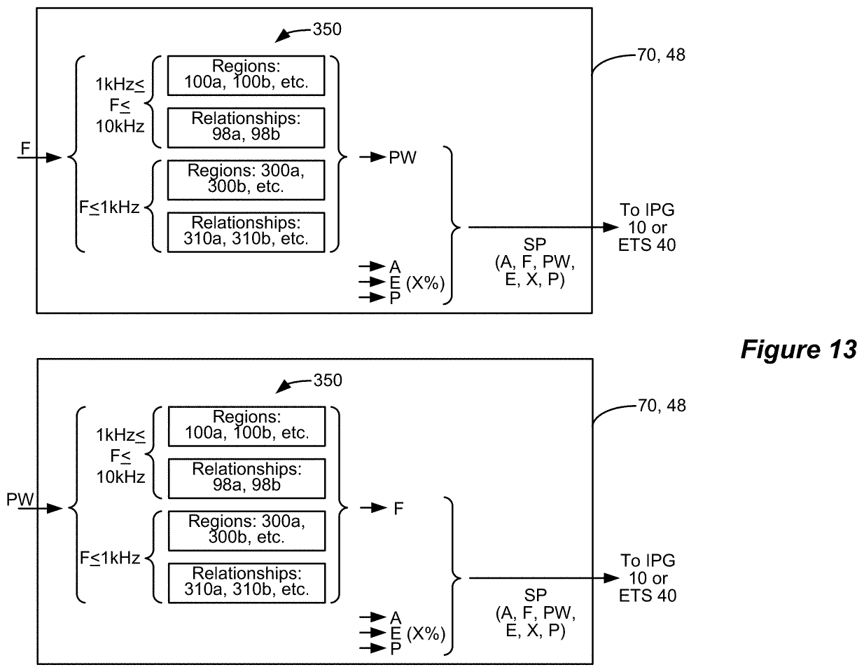

Once determined, the information 350 relating frequency and pulse width for optimal sub-perception therapy without paresthesia can be stored in an external device used to program the IPG 10 or ETS 40, such as the clinician programmer 50 or external controller 45 described earlier. This is shown in FIG. 13, in which the control circuitry 70 or 48 of the clinician programmer or external controller is associated with region information 100i or relationship information 98i for frequencies in the 1 kHz to 10 kHz range, and region information 300i or relationship information 310i for frequencies at or below 1 kHz. Such information can be stored in memory within or associated with the control circuitry. Storing of this information with the external device is useful to assisting the clinician with sub-perception optimization, as described further below. Alternatively, and although not shown, the information relating frequency and pulse width can be stored in the IPG 10 or ETS 40, thus allowing the IPG or ETS to optimize itself without clinician or patient input.

Information 350 can be incorporated into a fitting module. For example, fitting module 350 could operate as a software module within clinician programmer software 66, and may perhaps be implemented as an option selectable within the advanced 88 or mode 90 menu options selectable in the clinician programmer GUI 64 (FIG. 6). Fitting module 350 could also operate in the control circuitry of the IPG 10 or ETS 40.

The fitting module 350 can be used to optimize pulse width when frequency is known, or vice versa. As shown at the top of FIG. 13, the clinician or patient can enter a frequency F into the clinician programmer 50 or external controller 45. This frequency F is passed to the fitting module 350 to determine a pulse width PW for the patient, which is statistically likely to provide suitable pain relief without paresthesia. Frequency F could for example be input to the relationships 98i or 310i to determine the pulse width PW. Or, the frequency could be compared to the relevant region 100i or 300i within which the frequency falls. Once the correct region 100i or 300i is determined, F can be compared to the data in regions to determine a pulse width PW, which may perhaps be a pulse width between the PW+X and PW− X boundaries at the given frequency, as described earlier. Other stimulation parameters, such as amplitude A, active electrodes E, their relative percentage X %, and electrode polarity P can be determined in other manners, such as those described below, to arrive at a complete stimulation program (SP) for the patient. Based on the data from FIG. 9B, an amplitude near 3.0 mA might be a logical starting point, as this amplitude was shown to be preferred by patients in the 1 kHz to 10 kHz range. However, other initial starting amplitudes may be chosen as well, which amplitudes for sub-perception therapy may be dependent on frequency.

The bottom of FIG. 13 shows use of the fitting module 350 in reverse—that is to pick a frequency given a pulse width. Note that in the algorithms that follow or even when used outside of any algorithm, in one example, the system can allow the user to associate the frequency and pulse width such that when the frequency or pulse width is changed, the other of the pulse width or frequency is automatically changed to correspond to an optimal setting. In some embodiments, associating the frequency and pulse width in this manner can comprise a selectable feature (e.g., in GUI 64) useable when sub-perception programming is desired, and associating the frequency and pulse width can be unselected or unselectable for use with other stimulation modes. The above-incorporated '105 Publication discusses algorithms useable by fitting module 350 to provide sub-perception therapy to an SCS patient at frequencies of 10 kHz or lower.

As noted earlier, an interesting aspect of the disclosed trends and modeling of optimal sub-perception stimulation parameters is the realization that lower frequency stimulation can provide good results. The use of lower frequencies, as noted earlier, can result in stimulation parameters that require lower energy draws from the IPG. This was assessed earlier in several different ways. In FIG. 9B for example, it was explained for frequencies ranging from 1 kHz to 10 kHz that optimal sub-perception stimulation parameters (including pulse width and amplitude) result in mean charge per second (MCS) values that decline with frequency. In FIG. 11D, we see similarly for frequencies at and below 1 kHz that the total energy expended to produce optimal sub-perception stimulation decreases with frequency. In FIG. 11D, energy draw was represented by an assessment of the IPG's battery current (Ibat), and from the standpoint of a discharge time—i.e., the amount of time in which an IPG with a rechargeable battery can operate before it needs to be recharged. FIG. 11D shows that lower frequencies result in lower battery currents and longer discharge times, which reflects lower MCS values and lower energies used to form the optimal stimulation parameters.

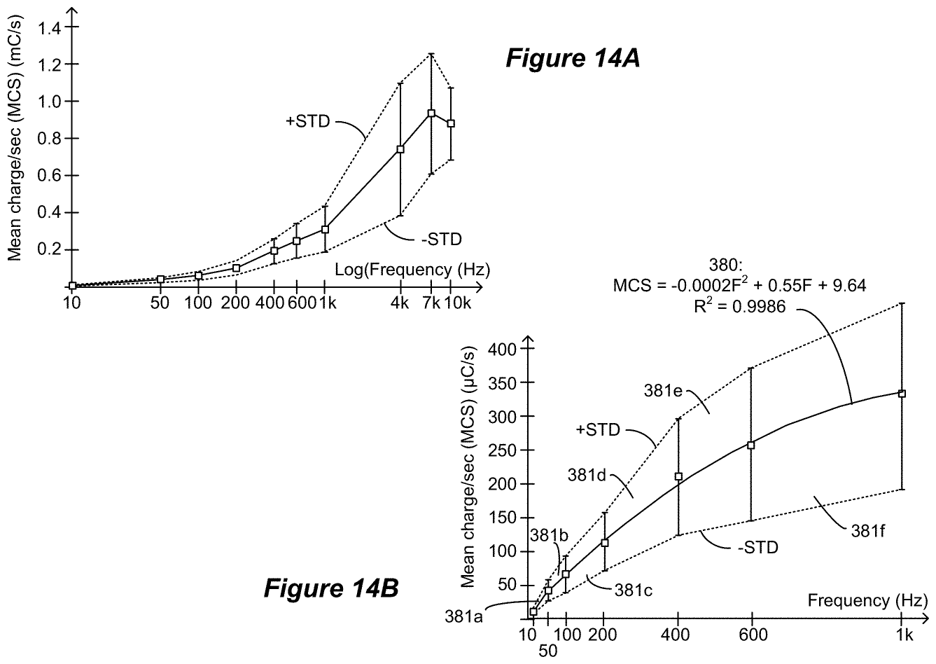

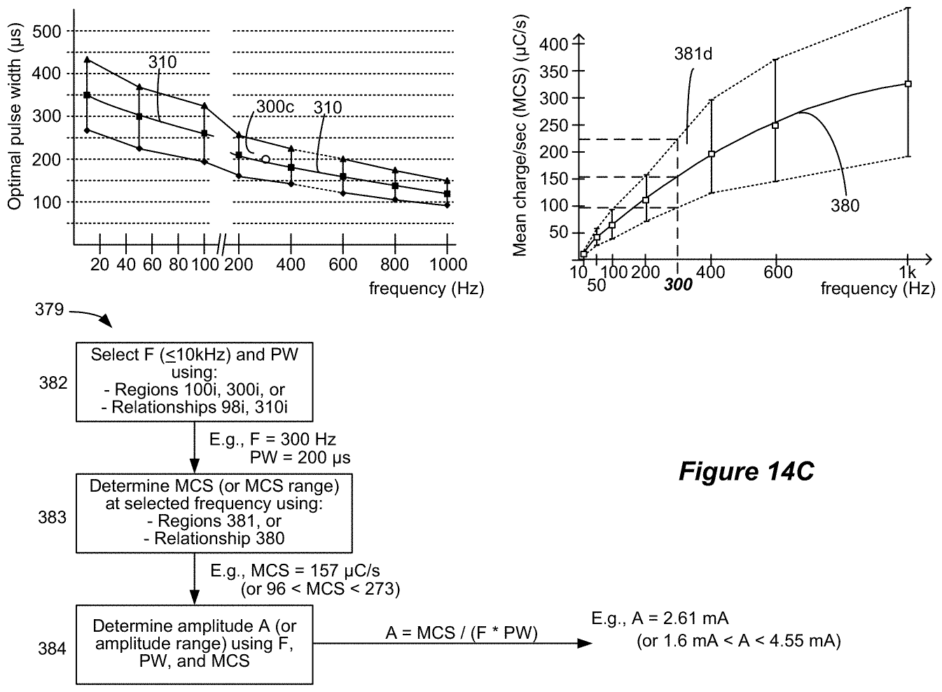

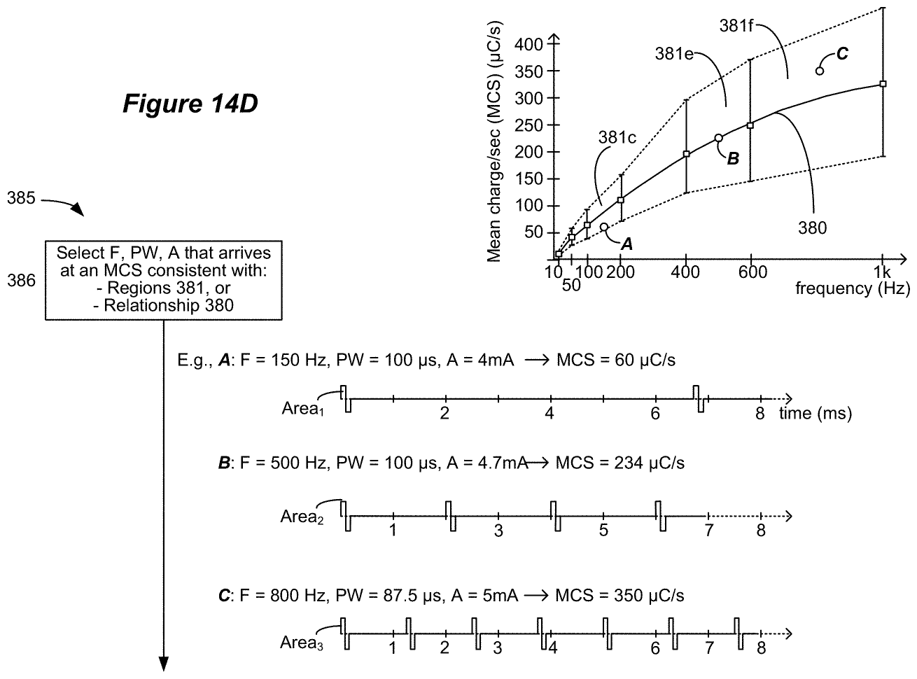

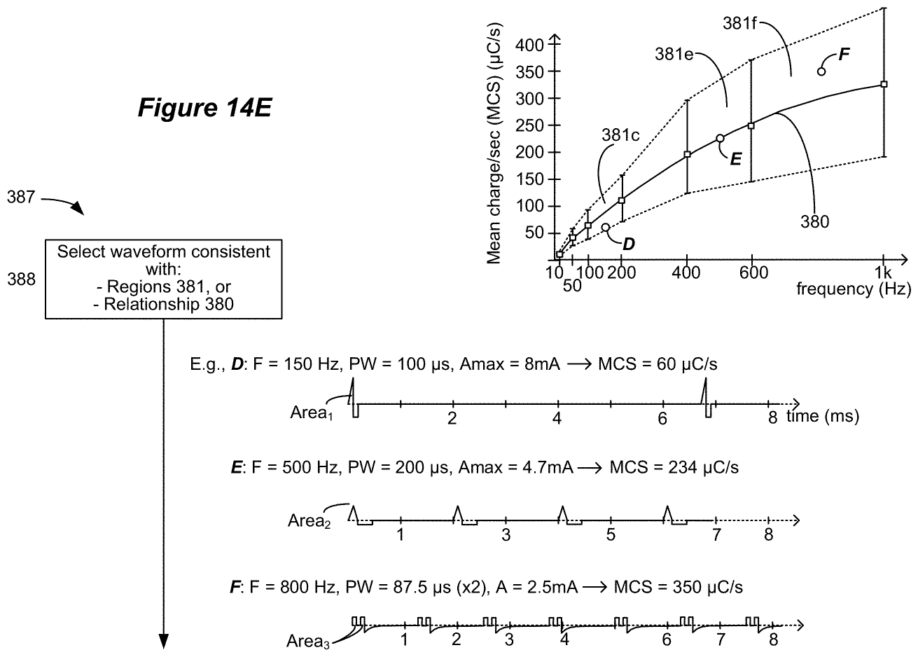

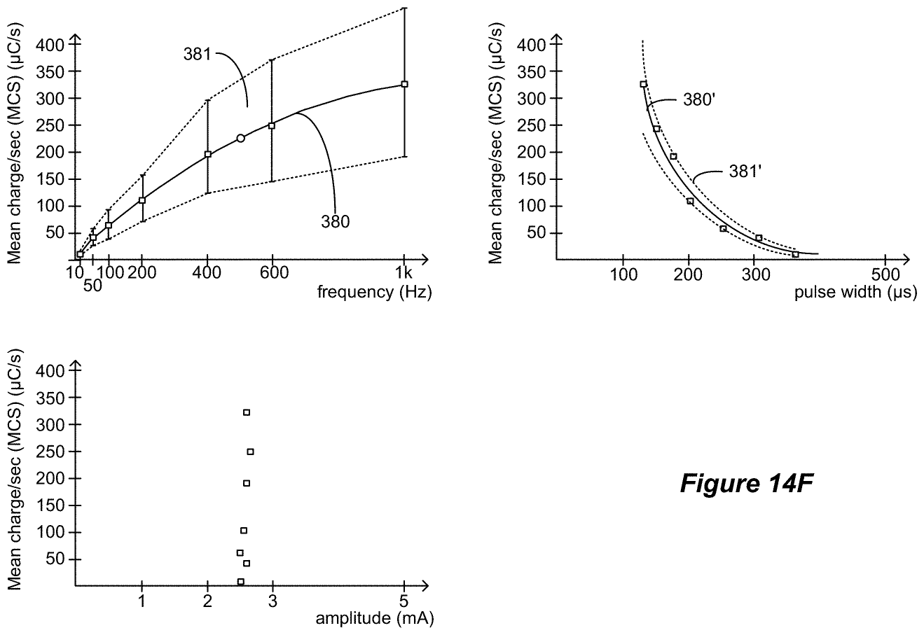

FIGS. 14A and 14B continue the analysis of neural dosing by again considering mean charge per second (MCS) data as a function of frequency for optimal stimulation parameters as taken from a population of patients. FIG. 14A shows MCS versus the logarithm of frequency for frequencies from 10 Hz to 10 kHz; FIG. 14B focuses more precisely on MCS data for a lower frequency range of 10 to 1 kHz. As noted earlier, MCS can be computed by multiplying the optimal frequency, pulse width, and amplitude for each of the patients tested. Because the optimal sub-perception stimulation parameters varied for each patient, error bars are associated with the MCS data in FIGS. 14A and 14B, which represent +/− one standard deviation (STD). As can be seen from both FIGS. 14A and 14B, the MCS data resulting from the optimal sub-perception parameters follows a predictable trend, and increases non-linearly with frequency, as discussed further below.

In FIG. 14B, the MCS data is analyzed with particularity for frequencies at 1 kHz and below, which as noted earlier is particularly interesting because such frequencies provide good sub-perception therapy results but at significantly lower energies. That being said, and although not shown, the data up to 10 kHz could also be analyzed and used in various algorithms such as those shown below, but this isn't shown. The data represented in FIG. 14B is shown in Table 4 below, which also shows the average optimal stimulation parameters for the patients tested:

TABLE 4

| | Pulse | Amplitude | Duty | MCS |
|---|---|---|---|---|
| Frequency | Width | (A) | cycle | (F*PW*A) |
| (F) | (PW) | (mA) ± | (PW*F) | (μC/s) ± |
| (Hz) | (μs) ± STD | STD | (%) | STD |
| 1000 | 122 ± 14 | 2.60 ± 0.90 | 12.2 | 326 ± 145 |
| 600 | 153 ± 20 | 2.66 ± 0.86 | 9.2 | 249 ± 108 |
| 400 | 183 ± 21 | 2.62 ± 0.86 | 7.3 | 196 ± 78 |
| 200 | 209 ± 28 | 2.59 ± 0.93 | 4.2 | 109 ± 42 |
| 100 | 252 ± 19 | 2.62 ± 0.80 | 2.5 | 64 ± 24 |
| 50 | 308 ± 25 | 2.54 ± 0.89 | 1.5 | 41 ± 14 |
| 10 | 350 ± 24 | 2.56 ± 0.83 | 0.35 | 9 ± 3 |

Although the MCS versus frequency data could be curve fit to other functions, the data fits well when modeled as a polynomial (relationship 380), which results in MCS=−0.0002F²+0.55F+9.64 (R²=0.9986), with MCS calculated in microCoulombs per second. Statistically-significant regions 381i around this relationship 380 can also be established, which region(s) may be defined by various error measures, as described above. In FIG. 14B, these regions 381i are bounded by the standard-deviation error bars. For example, at 10 Hz, MCS-STD=6 μC/s and MCS+STD=12 μC/s. At 50 Hz, MCS-STD=27 μC/s and MCS+STD=55 μC/s. Therefore, a region 381a that provides good sub-perception therapy is defined by the linearly bounded region of points (10 Hz, 6 μC/s), (10 Hz, 12 μC/s), (50 Hz, 55 μC/s), and (50 Hz, 27 μC/s). Table 4 defines the points that linearly bind each of the regions 381a-381f shown in FIG. 14B:

TABLE 5

| region | Bounded by points (Hz, μC/s) |
|---|---|
| 381a | (10, 6), (10, 12), (50, 55), (50, 27) |
| 381b | (50, 27), (50, 55), (100, 88), (100, 40) |
| 381c | (100, 40), (100, 88), (200, 151), (200, 67) |
| 381d | (200, 67), (200, 151), (400, 274), (400, 118) |
| 381e | (400, 118), (400, 274), (600, 357), (600, 141) |
| 381f | (600, 141), (600, 357), (1000, 471), (1000, 181) |

Particularly interesting are the results for frequencies equal to or below 400 Hz (e.g., regions 381a-d). Such frequencies yield particularly low energy values (i.e., low MCS values), and are not understood by the inventors to have been investigated in the art of sub-perception therapy. As mentioned elsewhere, conventional sub-perception therapies are understood to focus on higher stimulation frequencies.

FIGS. 14A and 14B shows that the deviation in MCS grows as frequency increases. It is hypothesized that this occurs because MCS is more sensitive as frequency increases to the step sizes that were used to adjust amplitude (0.1 mA) and pulse width (10 μs). This suggests when using higher frequencies, use of smaller step sizes may be advisable. In this regard, and although not shown in the figures, algorithms may be employed to adjust the step sizes of amplitude and pulse width as a function of frequency, with larger step sizes used for lower frequencies and smaller step sizes used for higher frequencies. Note that the ability to adjust step size may depend on the clinician programmer software used to adjust the stimulation parameters, and may also depend on the DAC circuitry used for a given patient.

The data in Table 4, which expresses the spread of the amplitude and pulse width data across the population of patients further suggests specific optimal sub-perception parameters. Such optimal stimulation parameters may be expressed in terms of a volume in frequency, amplitude, and pulse width. For example, at 10 Hz, A-STD=1.73 mA, A+STD=3.39 mA, PW-STD=326 μs, and PW+STD=374 μs. At 50 Hz, A-STD=1.65 mA, A+STD=3.43 mA, PW-STD=283 μs, and PW+STD=333 μs. Therefore, a volume that provides good sub-perception therapy is defined by the linearly bounded volume of the following eight points: (10 Hz, 1.73 mA, 326 μs), (10 Hz, 1.73 mA, 374 μs), (10 Hz, 3.39 mA, 326 μs), (10 Hz, 3.39 mA, 374 μs), (50 Hz, 1.65 mA, 283 μs), (50 Hz, 1.65 mA, 333 μs), (50 Hz, 3.43 mA, 283 μs), (50 Hz, 3.43 mA, 333 μs). Table 6 below sets forth these optimal stimulation parameter volumes:

TABLE 6

| volume | Bounded by points F, A, PW (Hz, mA, μs) |
|---|---|
| a | (10, 1.73, 326), (10, 1.73, 374), (10, 3.39, 326), (10, 3.39, 374), |
| | (50, 1.65, 283), (50, 1.65, 333), (50, 3.43, 283), (50, 3.43, 333) |
| b | (50, 1.65, 283), (50, 1.65, 333), (50, 3.43, 283), (50, 3.43, 333), |
| | (100, 1.82, 233), (100, 1.82, 271), (100, 3.42, 233), |
| | (100, 3.42, 271) |
| c | (100, 1.82, 233), (100, 1.82, 271), |
| | (100, 3.42, 233), (100, 3.42, 271), |
| | (200, 1.66, 181), (200, 1.66, 237), |
| | (200, 3.52, 181), (200, 3.52, 237) |
| d | (200, 1.66, 181), (200, 1.66, 237), |
| | (200, 3.52, 181), (200, 3.52, 237), |
| | (400, 1.76, 162), (400, 1.76, 204), |
| | (400, 3.48, 162), (400, 3.48, 204) |
| e | (400, 1.76, 162), (400, 1.76, 204), |
| | (400, 3.48, 162), (400, 3.48, 204), |
| | (600, 1.80, 133), (600, 1.80, 173), |
| | (600, 3.52, 133), (600, 3.52, 173) |
| f | (600, 1.80, 133), (600, 1.80, 173), |
| | (600, 3.52, 133), (600, 3.52, 173), |
| | (1000, 1.70, 108), (1000, 1.70, 136), |
| | (1000, 3.50, 108), (1000, 3.50, 136) |

The fact that the MCS of optimal stimulation parameters follows a predictable trend across patients suggests that such trend data can be used to select optimal stimulation parameters for a given patient, and FIGS. 14C-14E shows different manners or algorithms in which this could occur. As mentioned elsewhere, the algorithms shown could be implemented as software in an external device used to control a patient's IPG or ETS. The algorithms could also be implemented in the IPG or ETS themselves, which would allow optimal sub-perception stimulation parameters to be at least semi-automatically picked for the patient.

Algorithms to select optimal sub-threshold stimulation parameters may use the MCS data alone, or may also consider other modelling data established earlier, such as the relationship between frequency and pulse width (see, e.g., FIGS. 9A and 11A). Algorithm 379 in FIG. 14C uses such frequency versus pulse width data in step 382 to select a frequency and pulse width consistent with the various relationships (98i, 310i) or statistically-significant regions (100i, 300i) described earlier. An example of frequency versus pulse width relationship 310 and region 300c is reproduced in the left graph in FIG. 14C. In the example shown in FIG. 14C, a frequency of 300 Hz and 200 microseconds is selected at step 382, which point falls within region 300c (see FIG. 11A). Alternatively, a frequency and pulse width could also be selected using relationship 310 (see FIG. 11B).

Next, in step 383, a MCS value or range is determined using the selected frequency, and either relationship 380 or one of the regions 381i. For example, using relationship 380, an MCS value of 157 μC/s can be calculated when F=300 Hz. Alternatively, the error bars defining region 381d (in which F=300 Hz falls) allows an MCS range to be determined at this frequency, such as between approximately 96 and 273 μC/s, as shown in the right graph in FIG. 14C.

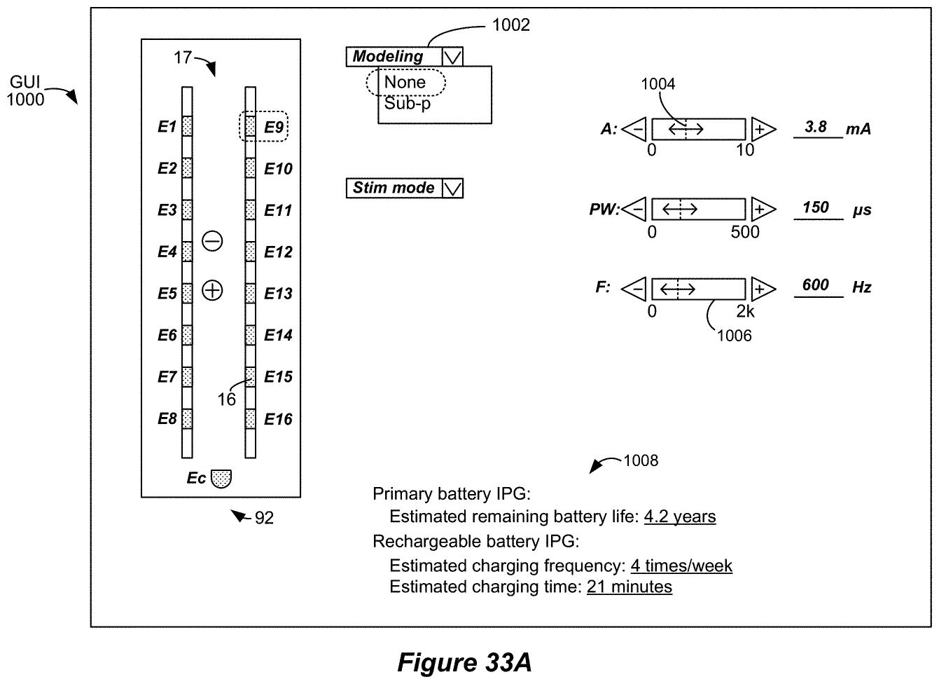

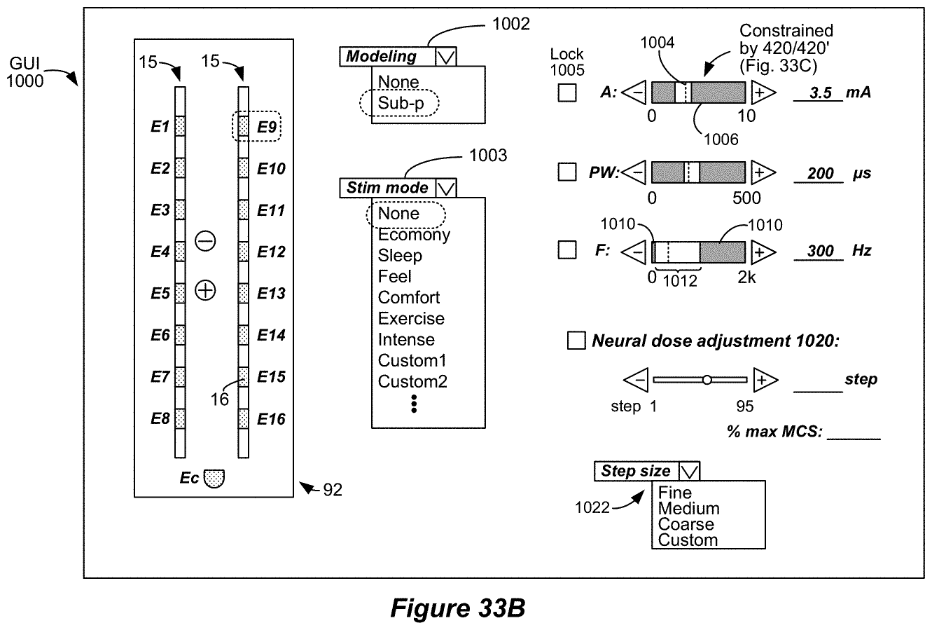

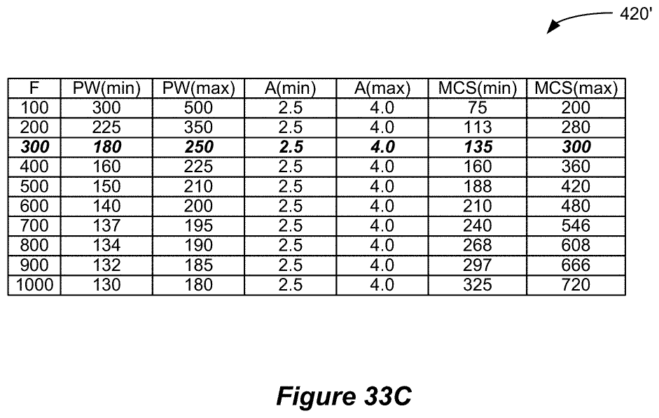

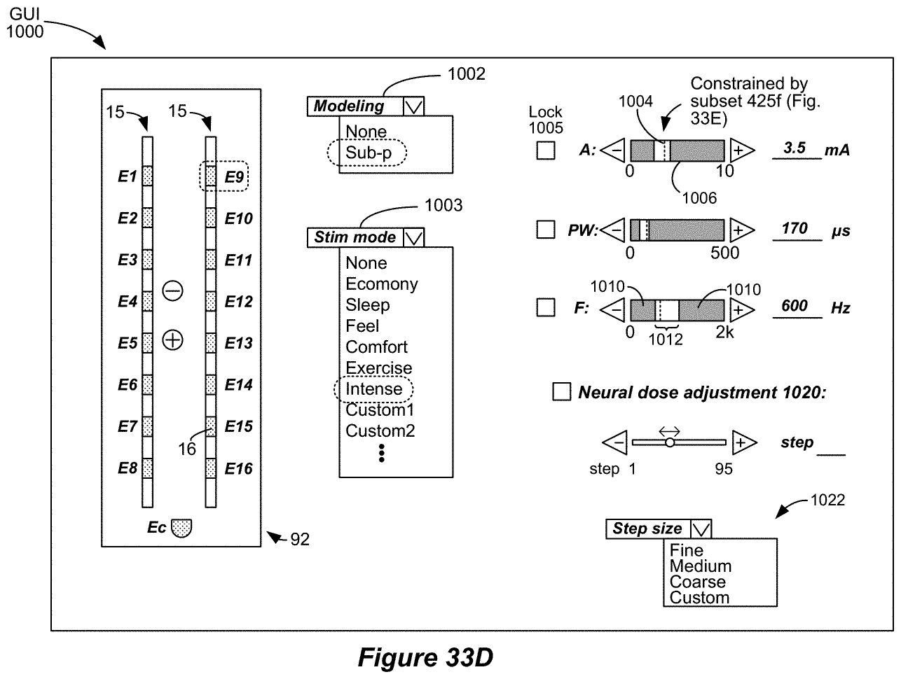

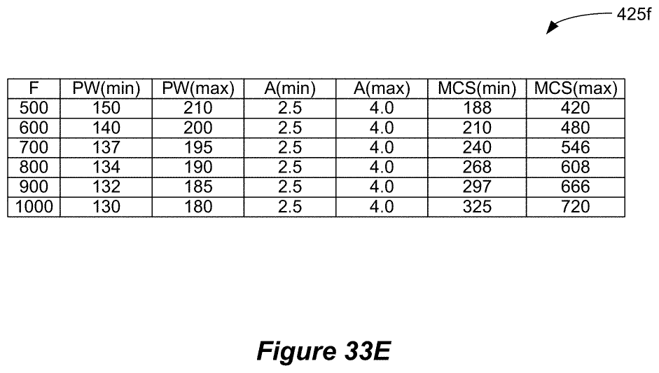

From the determined MCS value (or range), an amplitude value (or range) can be determined, as shown in step 384. This amplitude will comprise the MCS value (or range) divided by the product of the selected frequency (300 Hz) and pulse width (200 μs), which yields a single amplitude value of 2.61 mA, or an amplitude range from 1.6 to 4.55 mA, in the example shown. In short, algorithm 379 yields optimal sub-perception stimulation parameters, including frequency (e.g., 300 Hz), pulse width (e.g., 200 μs), and amplitude (e.g., 2.61 mA). Because the underlying data may reflect some spread (e.g., regions 300, 381) algorithm 379 could determine a plurality of potential candidate stimulation parameters sets to try with a given patient. The plurality of different stimulation sets may also be applied to the patient in a time-multiplexed manner. This is described in further detail later with respect to FIGS. 33A-35.

The trend of MCS versus frequency suggests that it may not be strictly necessary when selecting optimal sub-perception parameters to select the pulse width dependent on frequency (or vice versa), and FIG. 14D shows another example of an algorithm 385 that may be used to select optimal sub-perception stimulation parameters. In this example, the frequency v. pulse width trends (e.g., regions 100i, 300i, or relationships 98i and 310i) discussed earlier are not used. Instead, only the MCS versus frequency trends (regions 381i or relationship 380 are used), with the goal of selecting a frequency, pulse width, and amplitude that are consistent with such trend data. Operation of the algorithm 385 may select a single stimulation parameter set (i.e., a single frequency, pulse width, and amplitude), although FIG. 14D shows the selection of three such sets. Set ‘A’ comprises the set F=150 Hz, PW=100 μs, and A=4 mA, which results in a MCS value of 60 μC/s. This stimulation parameter set ‘A’ is shown on the graph in FIG. 14D, and notice that it is within statistically-significant region 381c, although it is not a point on relationship 380. Set ‘B’ comprises the set F=500 Hz, PW=100 μs, and A=4.7 mA, which results in an MCS value of 234 μC/s. This point is within region 381e, although it is also assumed that this comprises a point on relationship 380—i.e., when F=500 Hz, relationship 380 calculates MCS as 234. Set ‘C’ comprises the set F=800 Hz, PW=87.5 μs, and A=5 mA, which results in a MCS value of 350 μC/s. This stimulation parameter set ‘C’ is within region 381f, although it is not a point on relationship 380.

Also shown in FIG. 14D are the resulting waveforms for these different stimulation parameter sets. In this example, it is assumed that active charge recovery is used, and that the active charge recovery phase (the second phase of each pulse) is symmetric with the first phase (but of opposite polarity). However, this is not strictly necessary, and the charge recovery phase could comprise non-symmetric active phases, or passive phases, as described earlier, and as shown subsequently in the examples of FIG. 14E. Notice that these stimulation parameter sets may include frequencies and pulse widths that are consistent with the pulse width v. frequency trends explained earlier (100i, 300i, 98i, 310i), or they may not. Similar to algorithm 379, algorithm 385 may select a single stimulation parameter set for a given patient, or a plurality of such sets which can be tried, or applied in a time-multiplexed manner.

Because the MCS versus frequency data ultimately reflects an energy of the stimulation, other algorithms may determine waveforms for patient use that are not strictly defined by pulses, or at least are not strictly defined by constant frequencies, pulse widths, or amplitudes. Algorithm 387 in FIG. 14E selects a waveform which is consistent with the MCS versus frequency trend data (either region 381 or relationships 380), where the waveform has an irregular shape. For example, the pulses in the waveform selected by algorithm 387 may not have constant amplitudes, or may be sub-divided into a plurality of pulses.

For example, the pulses in stimulation parameter set ‘D’ have the same pulse width (100 μs) and frequency (150 Hz) as the pulses in set ‘A’ (FIG. 14C). However, the amplitude of the pulses is not constant, and instead ramps from zero to a maximum amplitude (Amax) of 8 mA over the pulse width. As such, the area (Area₁) under the pulses in set ‘D’ is equal to the area under the pulses in set ‘A’. Because MCS can also be defined as the pulse area times the frequency, the MCS of the pulses in sets ‘A and ‘D’ are the same (60 μC/s), which is again consistent with the MCS versus frequency trends noted (380, 381).

In another example, the pulses in stimulation parameter set ‘E’ have the same maximum amplitude (4.7 mA) and frequency (500 Hz) as the pulses in set ‘B’ (FIG. 14C). However, the pulse width is doubled (to 200 μs), and the amplitude ramps both up and down during the pulse width. As such, the area (Area₂) under the pulses in set ‘E’ is equal to the area under the pulses in set ‘B’, and both sets again have the same MCS value (234 μC/s), which is again consistent with MCS trend data.

Stimulation parameter set ‘F’ shows that each pulse can be formed by algorithm 387 as a group of pulses, sometimes known as a “burst” of pulses. In this example, the pulses are similar to those in set ‘C’ (FIG. 14C), and have the same pulse width (87.5 μs) and frequency (800 Hz). However, set ‘F’ creates a plurality of pulses (two, although there could be more) for each pulse shown in set ‘C’, and with half the amplitude (2.5 mA). The sum of the area under the two pulses in ‘F’ (Area₃) equals that under a single pulse in ‘C’, and so the pulses in ‘C’ and ‘F’ again have the same MCS value (350 μC/s), which is again consistent with MCS trend data. In short, algorithm 387 allows more generally for a waveform to be defined consistent with MCS trend data.

To this point, MCS trend data (e.g., 380, 381) has been illustrated as varying with frequency. However, MCS trend data as useful in the disclosed algorithms could also be defined with respect to other stimulation parameters. For example, and as shown in FIG. 14F, and as Table 4 above illustrates, MCS values predictive of good sub-perception therapy also varies in a predictable manner with pulse width, as shown in relationship 380′. This relationship 380′ could also be modeled (e.g., curve fit), and associated with error bars to determine regions 381′, which could then in turn be used in the algorithms just described to assist is selecting optimal sub-perception stimulation parameters. Shown also in FIG. 14F, and again taken from Table 4, is the variation in MCS values with amplitude. Notice here that the amplitude is fairly constant (e.g., around 2.6 mA) as MCS varies. While this trend may not be as useful in selecting stimulation parameters, notice that it is consistent with optimal sub-perception stimulation amplitudes reported earlier (see, e.g., FIG. 9B).

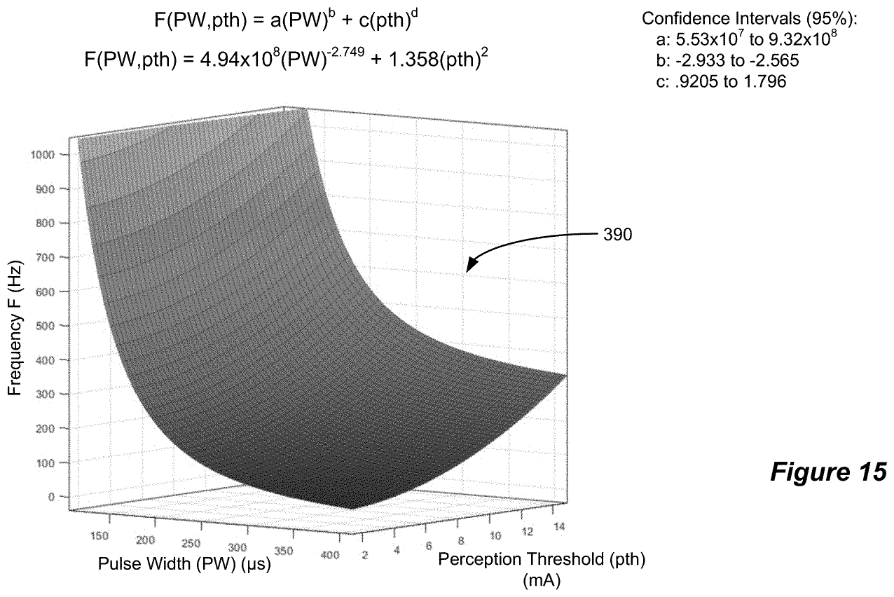

The results of further investigations are shown in FIGS. 15-20D, with the goal of providing optimal sub-perception modelling that takes into account perception threshold (pth) as well and frequency (F) and pulse width (PW). Perception threshold can be a significant factor to consider when modelling sub-perception stimulation, and using such modeling information to determine optimal sub-threshold stimulation parameters for each patient. Perception threshold, pth, comprises a lowest magnitude at which the patient can feel the effects of paresthesia (e.g., in mA), with magnitudes below this causing sub-perception stimulation. It is a reality that different patients will have different perception thresholds. Different perception thresholds result in significant part because the electrode array in some patients may be closer to spinal neural fibers than in other patients. Such patients will thus experience perception at lower magnitudes, i.e., pth will be lower for such patients. If the electrode array in other patients is farther from spinal neural fibers, the perception threshold pth will be higher. Improved modelling takes an understanding of pth into account, because the inclusion of this parameter can be used to suggest an optimal amplitude A for a patient's sub-perception stimulation in additional to optimal frequencies and pulse widths.

With this in mind, data was taken from patients to determine not only which frequencies and pulse widths they found optimal as described earlier, but also to determine the perception threshold at those frequencies and pulse widths. The resulting model 390 in shown in FIG. 15. This model 390 was determined based on testing with a sample of patients (N=25), with FIG. 15 showing mean values as determined by three-dimensional regression fitting, which yields model 390 as a surface in Frequency-Pulse Width-Perception Threshold space. Data as represented in FIG. 15 was taken at frequencies of 1 kHz and below. Data at these frequencies is of particular interest, because, as already mentioned, lower frequencies are more considerate of energy usage in an IPG or ETS, and hence is it particularly desirable to prove the utility of sub-perception stimulation in this frequency range. As can be seen by the equation in FIG. 15, data taken from the patient was modelled with a good fit by assuming that frequency varies with both pulse width (a(PW)b) and perception threshold pth (c(pth)d) in accordance with power functions. While these functions provided suitable fitting, other types of mathematical equations could be used for fitting as well. Model 390 as surface fit yields the following: F(PW,pth)=4.94×10⁸(PW)-2.749+1.358(pth)². Note that frequency, pulse width, and perception threshold are not simply proportionally related or inversely proportionately related model 390, but are instead related by non-linear functions. In another example, model 390 can be modeled as F(PW,pth)=a*exp(−(PW−b)/c)+d*pth².

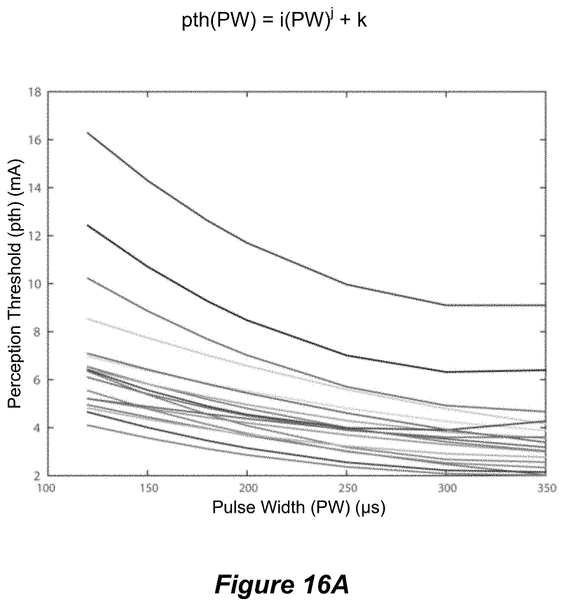

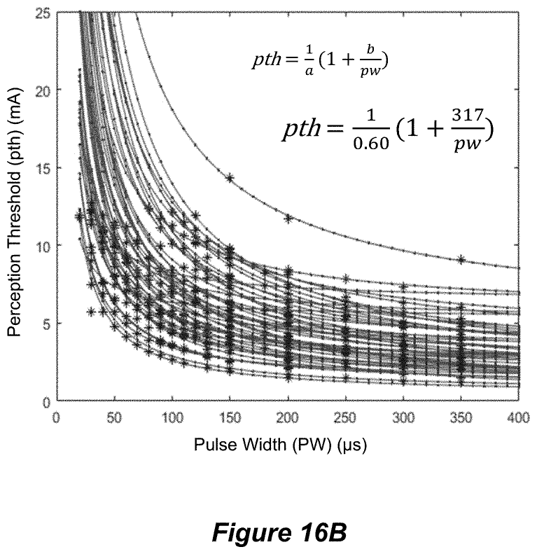

FIG. 16A shows further observations noticed from tested patients, and provides another modelling aspect that along with model 390 can be used to determine optimal sub-threshold stimulation parameters for a patient. FIG. 16A shows how perception threshold pth varies as a function of pulse width for the tested patients, with each patient being represented by a different line in the graph of FIG. 16A. Analysis of each of the lines suggests that the relationship between pth and PW can be well modeled with a power function, i.e., pth(PW)=i(PW)j+k, although again other mathematic functions could be used for fitting as well. The data of FIG. 16A was taken for each patient at a nominal frequency such as 200 to 500 Hz, with further analysis confirming that the results do not vary considerably with frequency (at least at frequencies of 150 Hz and higher, using biphasic pulses with active recharge). Pulse widths in FIG. 16A were limited to the range of approximately 100 to 400 microseconds. Limiting analysis to these pulse widths is reasonable, because previous testing (e.g., FIG. 11A) shows pulse widths in this range to have unique sub-perception therapeutic effectiveness at frequencies of 1 kHz and lower. FIG. 16B shows another example of pth versus pulse width for different patients, and shows another equation that can be used to model the data. Specifically, a Weiss-Lapicque, or strength-duration, equation is used in this example, which relates the amplitude and pulse width required to attain a threshold. The equation takes the form pth=(1/a)(1+b/PW), and when data for different patients are averaged, constants a=0.60 and b=317 result with good fitting results, where these values represent the mean constant parameters extracted from the population data.

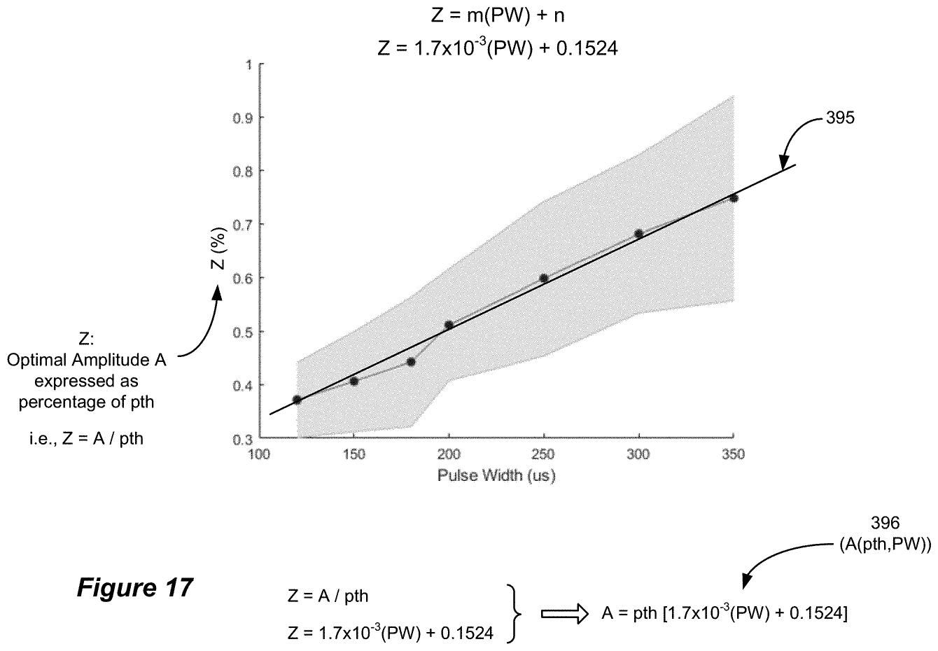

FIG. 17 shows still further observations noted from the tested patients, and provides yet another modeling aspect. FIG. 17 in effect shows how optimal sub-perception amplitudes A for patients vary in accordance with a patients' perception thresholds pth, as well as pulse width. In the graph in FIG. 17, the vertical axis plots a parameter Z, which relates a patients' perception thresholds pth and their optimal amplitudes A (which in a sub-perception therapy would be lower than pth). Specifically, Z is the optimal amplitude expressed as a percentage of pth, i.e., Z=A/pth. As FIG. 17 shows, Z varies with pulse width. At smaller pulse widths (e.g., 150 microsecond), Z is relatively low, meaning that the optimal amplitude A for patients was noted to be considerably lower than their perception thresholds (e.g., A=40% of pth). At longer pulse widths (e.g., 350 microseconds), Z is higher, meaning that the optimal amplitude A for patients was noted to be closer to their perception thresholds (e.g., A=70% of pth). Z and PW as noted from testing various patients generally have a linear relationship over the pulse widths tested, and so linear regression was used to determine their relationship, yielding Z=0.0017(PW)+0.1524 (395). Again, testing in FIG. 17 was limited to the general range of 100 to 400 microseconds noted to be useful for sub-perception therapy at less than 1 kHz. It might be expected that testing over a wider pulse width range (e.g., less than 100 microseconds, or greater than 400 microseconds) would show some variance from the linear relationship noted. For example, Z might level off to some value smaller than 1 for pulse widths higher than 400 microseconds, and might level off to a value greater than 0 for pulse widths shorter than 100 microseconds. Because Z varies with pulse width as curve fit, and because Z also varies with optimal amplitude A and perception threshold pth (Z=A/pth), the modelling of FIG. 17 allows optimal amplitude A to be modelled as a function of both perception threshold pth and pulse width PW, i.e., A=pth [0.0017(PW)+0.1524] (396). The inventors observe that optimal amplitude A is generally invariant to changes in frequency and pulse width. However, perception threshold varies with pulse width. Thus, Z varies with pulse width, while optimal amplitude A may not.

Recognizing and modeling these observations, the inventors have developed an algorithm 400 that can be used to provide personalized sub-perception therapy for particular patients. This algorithm 400 can largely be implemented on the clinician programmer 50, and results in the determination of a range of optimal sub-perception parameters (e.g., F, PW, and A) for the patient. Preferably, as last step in the algorithm 400, the range or volume of optimal sub-perception parameters is transmitted to the patient's external controller 45 to allow the patient to adjust their sub-perception therapy within this range or volume.

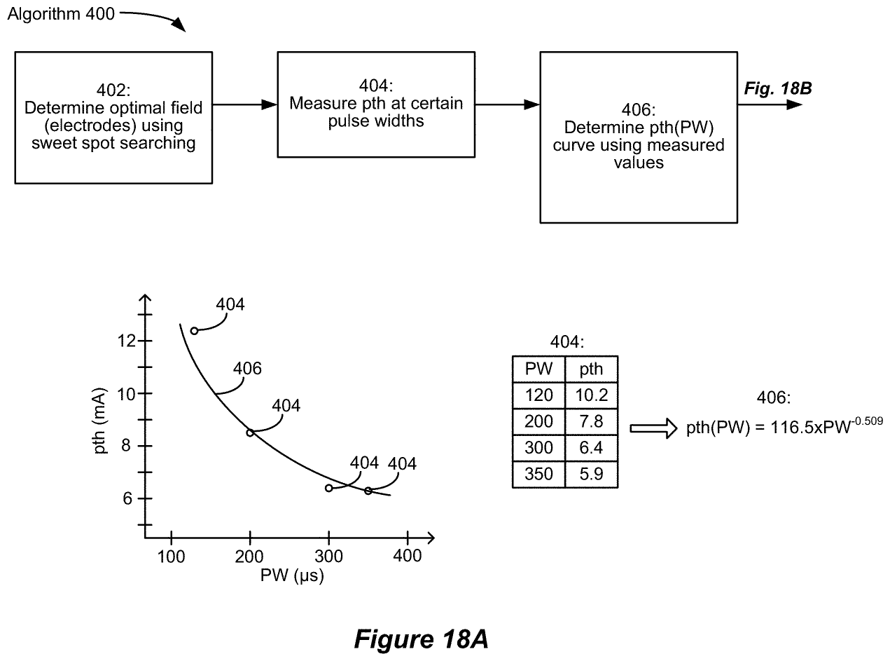

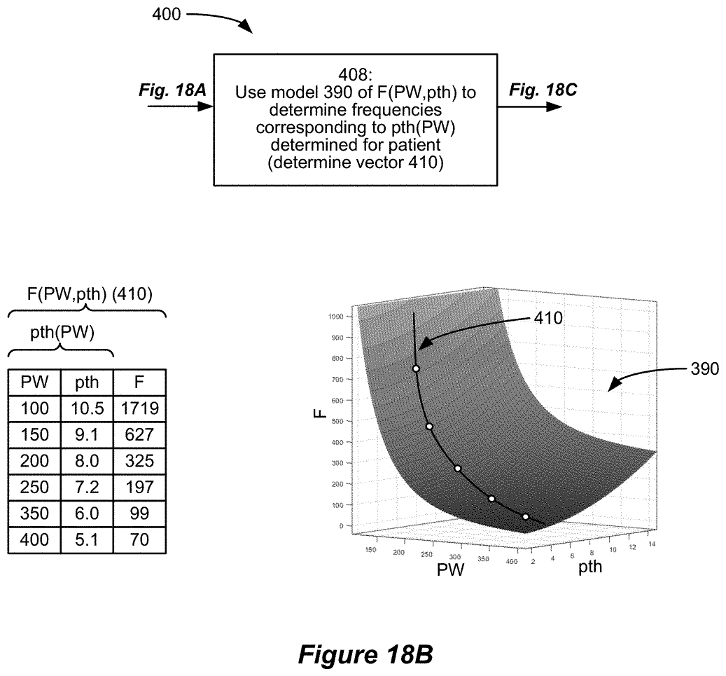

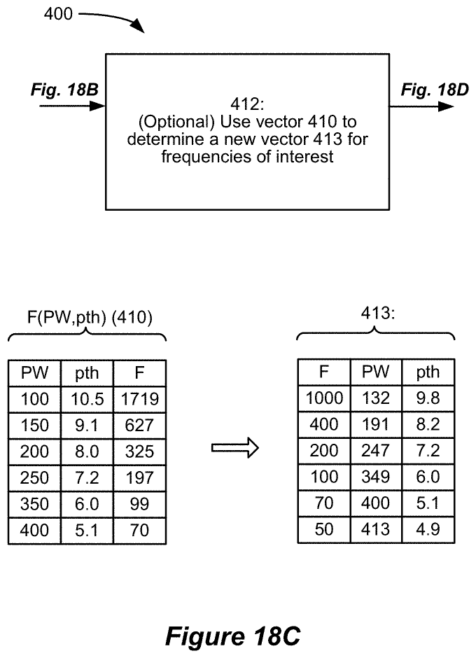

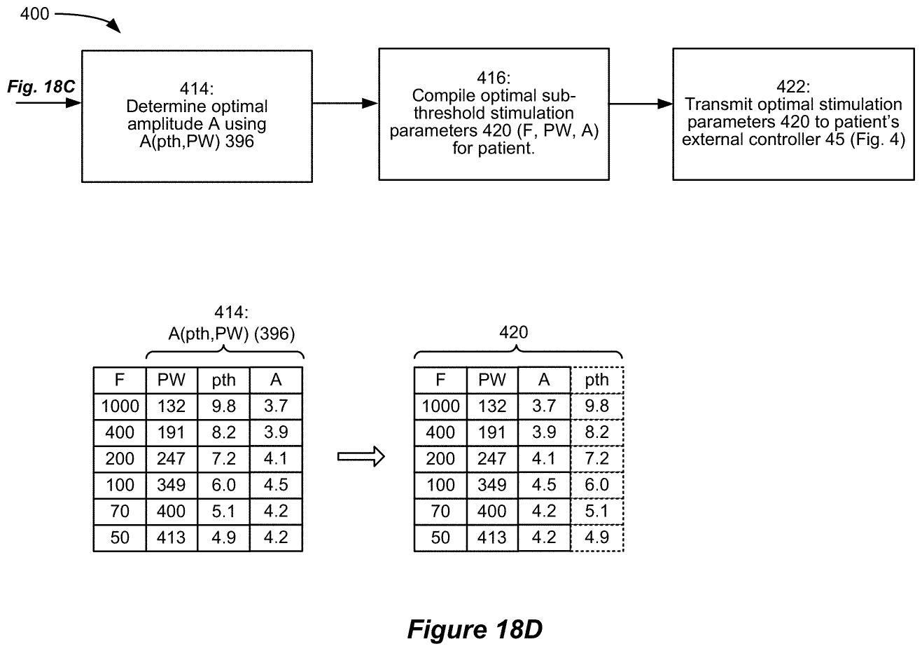

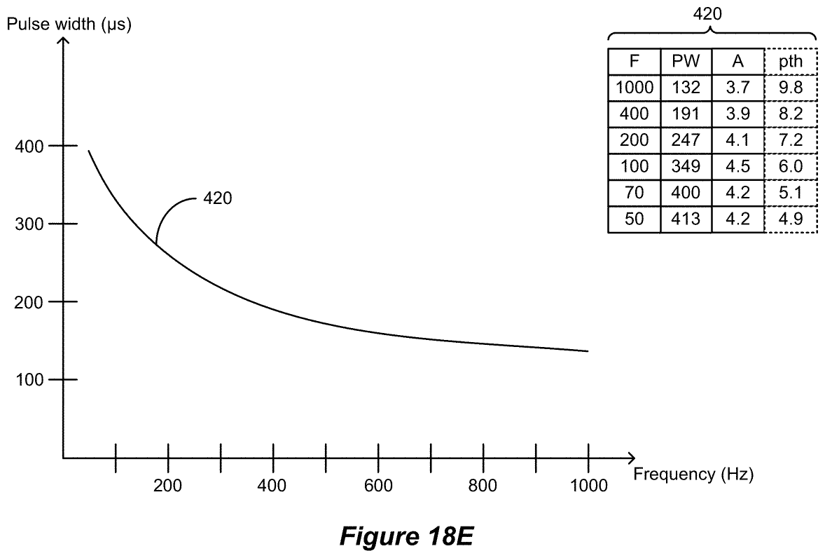

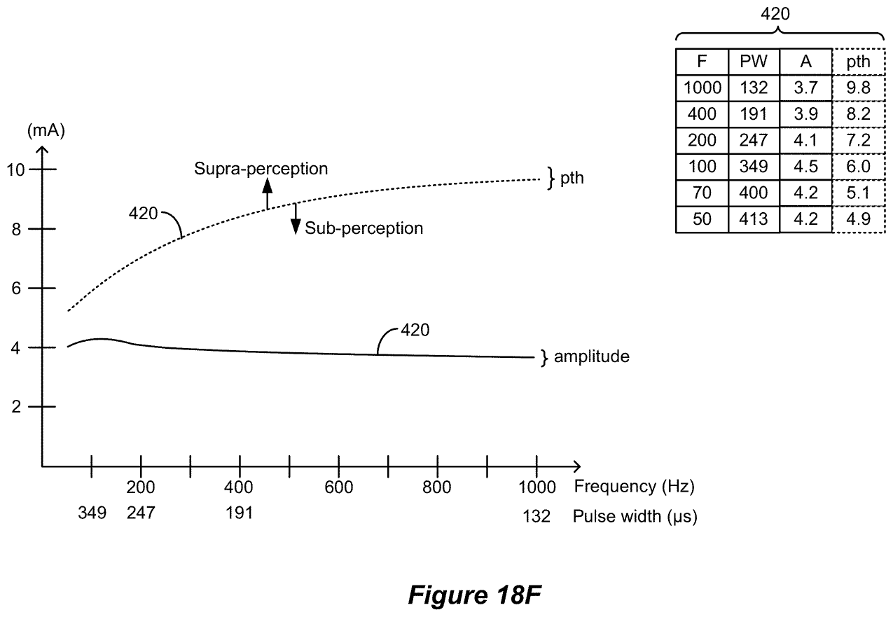

The algorithm 400, shown starting in FIG. 18A, starts in step 402 by determining for a given patient the sweet spot in the electrode array at which therapy should be applied—i.e., by identifying which electrodes should be active and with what polarities and percentages (X %). The results of sweet spot searching may already be known for a given patient, and thus step 402 should be understood as optional. Step 402 and subsequent steps may be accomplished using clinician programmer 50.

At step 404, a new patient is tested by providing situation pulses, and in the algorithm 400, such testing involves measuring the patient's perception threshold pth at various pulse widths during a testing procedure, using the sweet spot electrodes already identified at step 402. As discussed earlier with respect to FIGS. 16A and 16B, testing of different pulse widths can occur at a nominal frequency such as in the range of 200 to 500 Hz. Determining pth at each given pulse width involves applying the pulse width, and gradually increasing the amplitude A to a point where the patient reports feeling the stimulation (paresthesia), resulting in a pth expressed in terms of amplitude (e.g., milliamps). Alternatively, determining pth at each given pulse width can involve decreasing the amplitude A to a point where the patient reports no longer feels the stimulation (sub-threshold). Testing 404 of a particular patient is shown graphically and in tabular form in FIG. 18A. Here, it is assumed that the patient in question has a paresthesia threshold pth of 10.2 mA at a pulse width of 120 microseconds; a pth of 5.9 mA at a pulse width of 350 microseconds, and other values between these.

Next, in step 406, the algorithm 400 in the clinician programmer 50 models the pth v. PW data points measured in step 404, and curve fits them to a mathematical function. This mathematical function could be one noticed earlier to well model pth and PW in other patients, such as a power function pth(PW)=i(PW)^{j}+k or the Weiss Lapicque equation, as discussed earlier with respect to FIGS. 16A and 16B. However, any other mathematical function could be used to curve fit the current patient's data measurements, such as a polynomial function, and exponential function, etc. In the illustrated data, a power function well models the data, yielding pth(PW)=116.5×PW^{-0.509}. (For simplicity, constant ‘k’ has been ignored). The measured data in table 404, as well as the determined curve-fit relationship pth(PW) 406 determined for the patient may be stored in memory in the clinician programmer 50 for use in subsequent steps.

Next, and referring to FIG. 18B, algorithm 400 proceeds to compare the pth(PW) relationship determined in step 406 to the model 390. This is explained with reference to a table shown in FIG. 18B. In this table, values for pth and PW are populated, as determined by the pth(PW) (406) determined in FIG. 18A. As can be seen, discrete pulse width values of interest (100 microseconds, 150 microseconds, etc.) may be used (which may vary from the exact pulse widths used during patient testing in step 404). While only six rows of PW v. pth values are shown in the table of FIG. 18B, this could be a much longer vector of values, with pth determined at discrete PW steps (such as 10 microsecond steps).

The pth v. PW values (from function 406) are in step 408 compared against the three-dimensional model 390 to determine frequencies F that would be optimal at these various pth v. PW pairs. In other words, the pth and PW values are provided as variables into the surface fit equation (F(PW,pth)) 390 in FIG. 15 to determine optimal frequencies, which frequencies are also shown as populated into the chart in FIG. 18B. At this point, the table in FIG. 18B represents a vector 410 relating pulse widths and frequencies that are optimal for the patient, and that in addition include the perception threshold for the patient at these pulse width and frequencies values. In other words, a vector 410 represents values within the model 390 that are optimal for the patient. Note that vector 410 for the patient can be represented as a curved line along the three-dimensional model 390, as shown in FIG. 18B.

Next, and as shown in step 412 in FIG. 18C, the vector 410 can optionally be used to form another vector 413, which contains values of interest or more practically values that may be supportable by the IPG or ETS. For example, notice that vector 410 for the patient includes frequencies at higher values (e.g., 1719 Hz), or otherwise at odd values (such as 627 and 197 Hz). Frequencies at higher values may not be desirable to use, because even if effective for the patient, such frequencies will involve excessive power draws. See, e.g., FIG. 11D. Moreover, the IPG or ETS at issue may only be able to provide pulses with frequencies at discrete intervals (such as in 10 Hz increments). Therefore, in vector 413, frequencies of interest or that are supported are chosen (e.g., 1000 Hz, 400 Hz, 200 Hz, 100 Hz, etc.), and then corresponding values for PW and pth are interpolated using vector 410. Although not shown, it may be useful to formulate vector 410 as an equation F(PW,pth)) to make vector 413 easier to populate. Nonetheless, vector 413 includes essentially the same information as vector 410, albeit at desirable frequencies. Realize that only certain pulse widths may be supportable by the IPG or ETS (e.g., in 10 microsecond increments). Therefore, the pulse widths in vector 413 may be adjusted (e.g., rounded) to nearest supported values, although this isn't shown in the drawings.

Next, and referring to FIG. 18D, the algorithm 400 in step 414 determines optimal amplitudes for the pulse width and pth values in vector 413 (or vector 410 if vector 413 isn't used). This occurs by using the amplitude function 396 determined earlier in FIG. 17, i.e., A(pth,PW). Using this function, an optimal amplitude A can be determined for each pth, PW pair in the table.

At this point, in step 416, optimal sub-threshold stimulation parameters F, PW, A 420 are determined as a model specific to the patient. Optimal stimulation parameters 420 may not need to include the perception threshold, pth: although pth was useful to determine optimal subthreshold amplitude A for the patient (step 414), it may no longer be a parameter of interest as it is not a parameter that the IPG or ETS produces. However, in other examples discussed later, it can be useful to include pth with the optimal parameters 420, as this can allow a patient to adjust their stimulation to a supra-perception level if desired. At this point, optimal stimulation parameters 420 may then be transmitted to the IPG or ETS for execution, or as shown in step 422, they may be transmitted to the patient's external controller 45, as described next.

FIGS. 18E and 18F depict optimal parameters 420 in graphical form. While optimal parameters 420 in this example comprise a three-dimensional range or line of coordinates (F, PW, and A), they are depicted in two two-dimensional graphs for easier illustration: FIG. 18E shows the relationship between frequency and pulse width, and FIG. 18F shows the relationship between frequency and amplitude. Note also that FIG. 18F shows the paresthesia threshold pth, and additionally shows on the X-axis the pulse width corresponding to the various frequencies from FIG. 18E. Note that the shapes of the data on these graphs could vary from patient to patient (e.g., based on the pth measurements of FIG. 18A), and could also change depending on the underlying modelling used (e.g., FIGS. 15-17). The various shapes of the trends shown thus should not be construed as limiting.

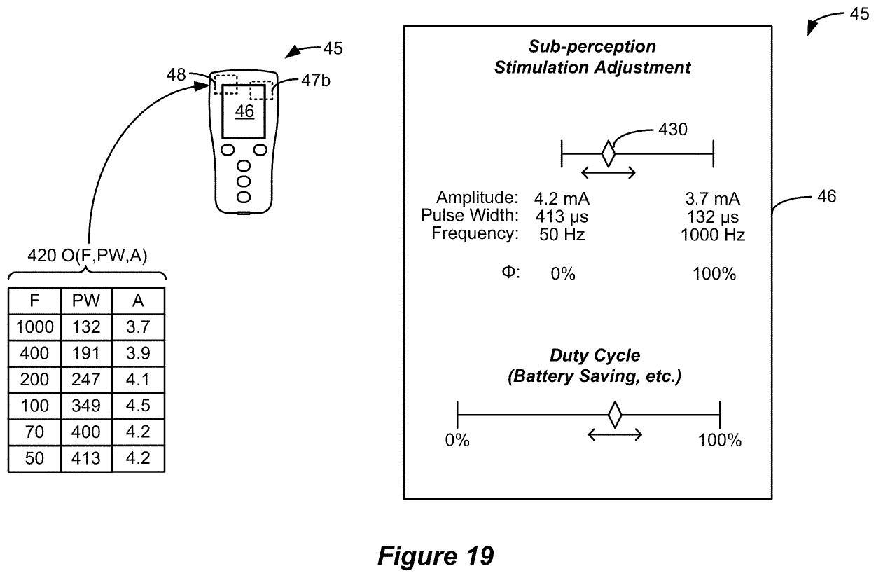

The optimal stimulation parameters 420 determined by the algorithm 400 comprise a range or vector of values, comprising frequency/pulse width/amplitude coordinates that based on modeling (FIGS. 15-17), and on testing of the patient (step 404, FIG. 18A), will result in sub-threshold stimulation that is optimal for that patient. While optimal parameters 420 are shown for simplicity in tabular form in FIG. 19, it should be understood these optimal parameters (O) may be curve fit using an equation that includes frequency, pulse, and amplitude (i.e., O=f(F,PW,A)). Because each of these coordinates are optimal, it may be reasonable to allow the patient to use them with their IPG or ETS, and as a result the optimal parameters 420 may be sent from the clinician programmer 50 to the patient external controller 45 (FIG. 4) to allow the patient to select between them. In this regard, the optimal parameters 420, whether in tabular form or in the form of an equation, can be loaded into control circuitry 48 of the external controller 45.

Once loaded, the patient can access a menu in the external controller 45 to adjust the therapy the IPG or ETS provides consistent with these optimal parameters 420. For example, FIG. 19 shows a graphical user interface (GUI) of the external controller 45 as displayed on its screen 46. The GUI includes means to allow the patient to simultaneously adjust the stimulation within the range of determined optimal stimulation parameters 420. In one example, a slider is included in the GUI with a cursor 430. The patient may select the cursor 430 and in this example move it to the left or right to adjust the frequency of stimulation pulses in their IPG or ETS. Moving it to the left reduces the frequency down to a minimum value included in the optimal parameters 420 (e.g., 50 Hz). Moving the cursor 430 to the right increases the frequency to a maximum values included in the optimal parameters (e.g., 1000 Hz). As the cursor 430 is moved and the frequency of stimulation is changed, the pulse widths and amplitudes are simultaneously adjusted as reflected in optimal parameters 420. For example, at F=50 Hz, the amplitude is automatically set to A=4.2 mA, and the pulse width is set to 413 microseconds. At F=1000 Hz, the amplitude is set to A=3.7 mA, and the pulse width is set to 132 microseconds. In effect the cursor 430 allows the patient to navigate through the optimal parameters 420 to find a F/PW/A setting they prefer, or simply to choose stimulation parameters that are still effective but require lower power draws from the IPG or ETS (e.g., at lower frequencies). Note that the frequency, pulse width, and amplitude may not be adjusted proportionately or inversely proportional with respect to each other but will follow non-linear relationships in accordance with the underlying modelling.

In another example, it may be useful to allow the patient to adjust stimulation without knowledge of the stimulation parameters, i.e., without displaying the parameters, which may be too technical for the patient to understand. In this regard, the slider can be labeled with a more generic parameter, such as φ, which the patient can adjust, such as between 0 and 100%. The three-dimensional simulation parameters A, PW, and F can be mapped to this one-dimensional parameter φ (e.g., 4.2 mA, 413 μs, and 50 Hz can equal 0% as shown). Generally speaking, the patient may understand parameter φ as a sort of “intensity” or “neural dose” which is higher at higher percentages. This may in fact be true given depending on the manner in which the optimal stimulation parameters 420 are mapped to φ. In another modification not shown, the optimal stimulation parameters can be selected using a mean charge per second (MCS) model (e.g., 380, 381), as described earlier with respect to FIGS. 14A-14F. The GUI can then also allow the user to navigate within a MCS model to select optimal stimulation parameters.

It should be appreciated that while the GUI of the external controller 45 does allow the patient some flexibility to modify stimulation parameters for his IPG or ETS, it is also simple, and beneficially allows the patient to adjust all three stimulation parameters simultaneously using a single user interface element, all while being ensured that the resulting stimulation parameters will provide optimal sub-threshold stimulation.

Other stimulation adjustment controls may be provided by the external controller 45 as well. For example, as shown in FIG. 19, another slider can allow the patient to adjust the duty cycle to control the extent to which pulses will be continually running (100%) or completely off (0%). A duty cycle in the middle (e.g., 50%) will mean that pulses will run for a period of time (from second to minutes) and will then be off for that exact same duration. Because “duty cycle” may be a technical concept that a patient would not intuitively understand, note that the duty cycle may be labeled in a more intuitive manner. Thus, and as shown, the duty cycle adjustment may be labeled differently. For example, because lower duty cycles would affect lower power draws, the duty cycle slider may be label as a “battery saving” feature, as a “total energy” feature, a “total neural charge dose” feature, or the like, which may be easier for the patient to understand. Duty cycling may also comprise a feature in the external controller 45 that is locked to the patient, and only made accessible to a clinician for example, upon entering an appropriate password or other credentials. Note that the duty cycle could be smoothly adjusted, or made adjustable in pre-set logical increments, such as 0%, 10%, 20%, etc. Duty cycle adjustment is not show in subsequent user interface examples for simplicity, but could be used in such examples as well.

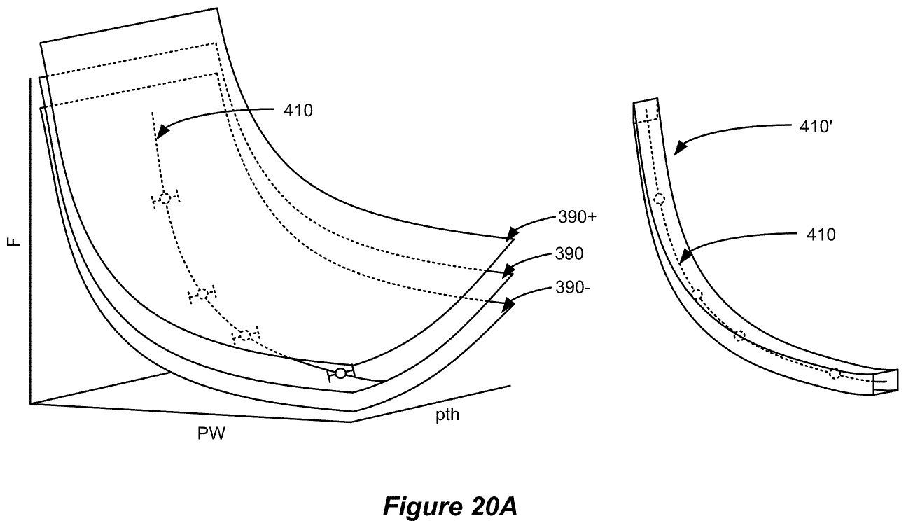

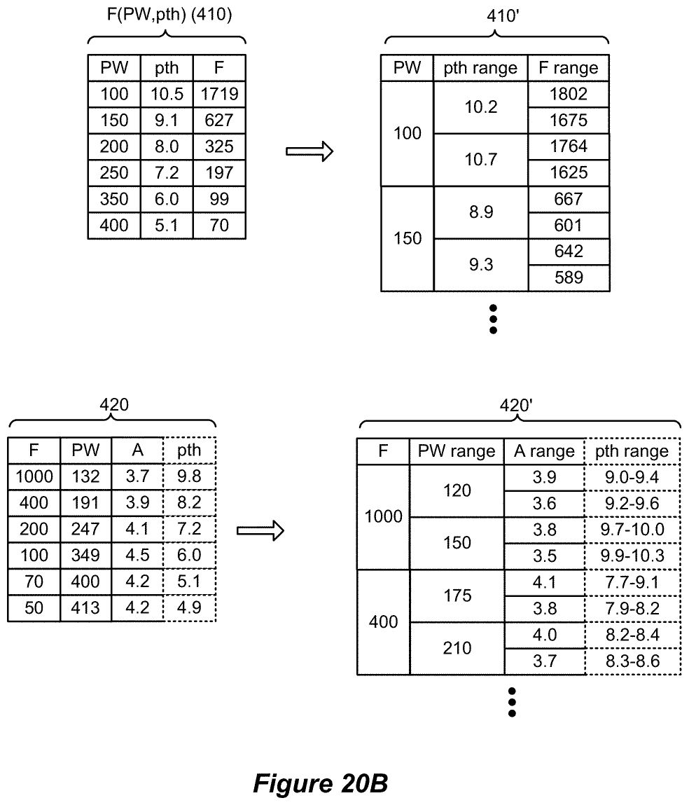

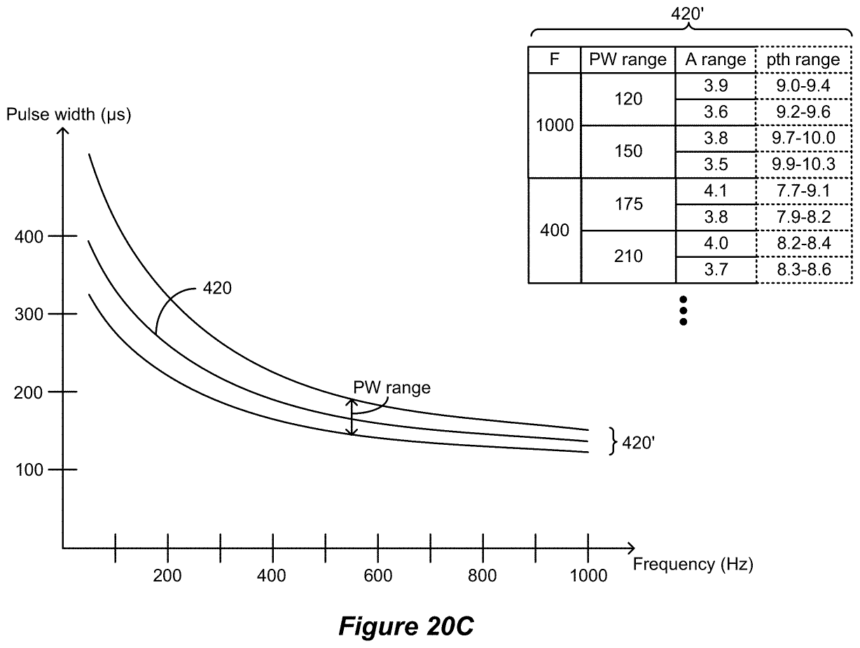

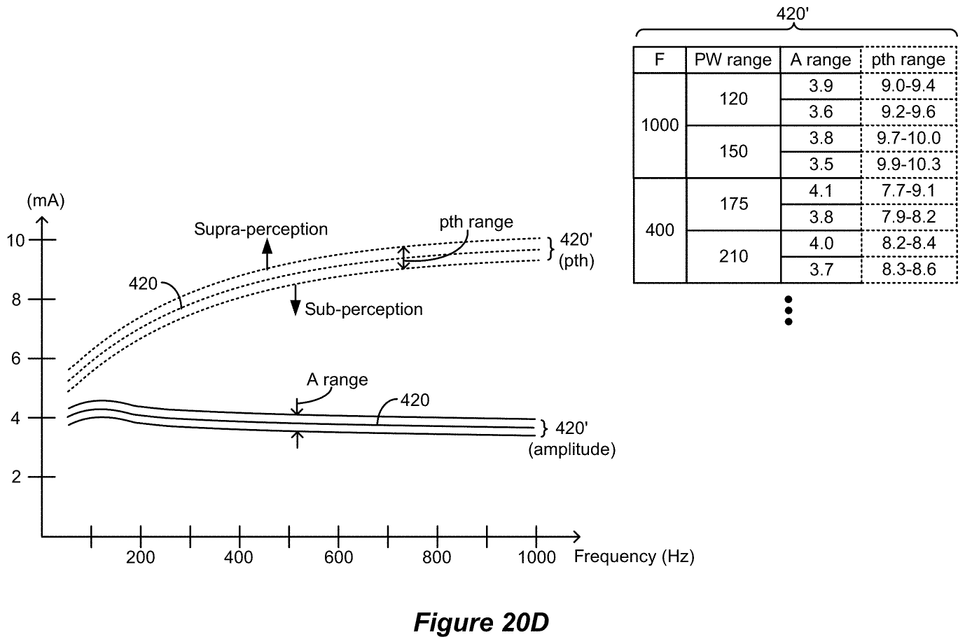

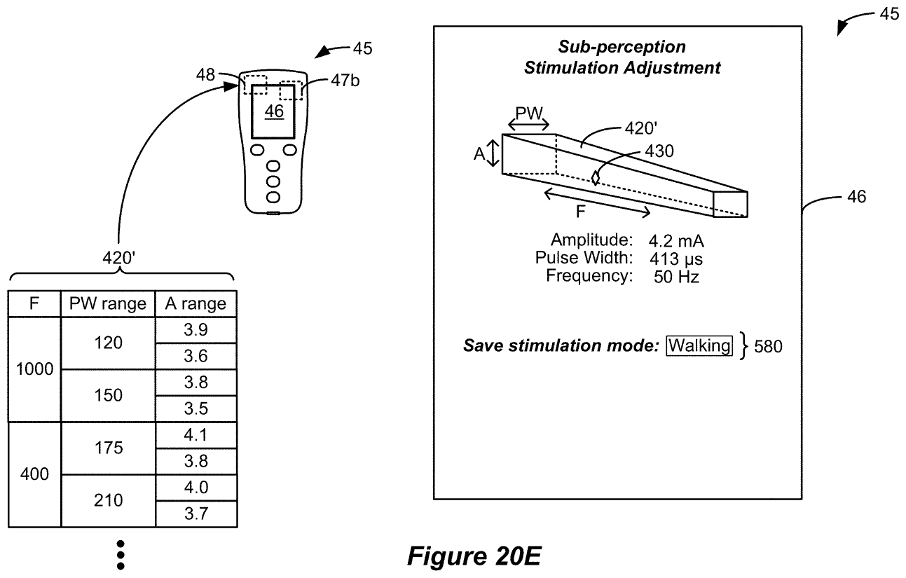

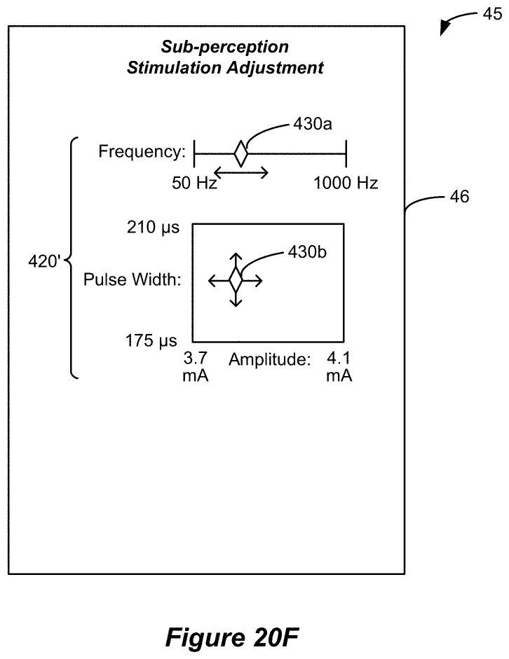

FIGS. 20A-20D address the practicality that the modeling leading to the determination of optimal parameters 420 may not be perfect. For example, model 390—modeling frequency as a function of PW and pth (F(PW,pth)); FIG. 15)—is averaged from various patients, and can have some statistical variance. This is illustrated simply in FIG. 20A by showing surfaces 390+ and 390—that are higher and lower from the mean as reflected in surface model 390. Surfaces 390+ and 390—may represent some degree of statistical variance or error measure, such as plus or minus one sigma, and may in effect generically comprise error bars beyond which the model 390 is no longer reliable. These error bars 390+ and 390—(which may not be constant over the entire surface 390) can also be determined from an understanding of the statistical variance in the various constants assumed during modeling. For example, values a, b, c, and d in model 390 may be determined with different measures of confidence. As shown in FIG. 15, constants a, b, and c vary within a 95% confidence interval. For example, constant ‘a’ can range from 5.53×10⁷ to 9.32×10⁸, as shown on the graph. (Here, it is assumed that constant d is simply 2 and does not vary). Likewise, values used to model the relationship of pth and pulse width (FIGS. 16A and 16B) may have different measure of confidence, as may values m and n used to model the relationship between optional amplitude, pth, and PW (FIG. 17). Over time, as data is taken on more patients, it would be expected that the confidence of these models would improve. In this regard, note that algorithm 400 can easily be updated with new modeling information from time to time by loading new modeling information into the clinician programmer 50.

Statistical variance means that optimal stimulation parameters may not comprise discrete values, but may instead fall within a volume. This is illustrated in FIG. 20A as concerns the vector 410 determined for the patient (see FIG. 18B). Given statistical variance, vector 410 may comprise a rigid line within a volume 410′. In other words, there may not be a one-to-one correspondence of PW, pth, and F, as was the case for vector 410 in FIG. 18B. Instead, for any given variable (such as pulse width), the pth as determined for the patient (using the pth(PW) model in step 406) may vary in a range between statistically-significant maximum and minimum values, as shown in FIG. 20B. Statistical variation in model 390 (FIG. 15) may also mean that maximum and minimum frequencies may be determined for each maximum and minimum pth in step 408. As this trickles through the algorithm 400, the optimal stimulation parameters 420 may also not have one-to-one correspondence between frequency, pulse width, and amplitude. Instead, and as shown in FIG. 20B, for any frequency, there may be a range of maximum and minimum optimal pulse width of statistical significance, and a likewise a range of optimal amplitudes A. Effectively, then, optimal stimulation parameters 420′ may define a statistically-significant volume of coordinates in Frequency-Pulse Width-Amplitude space rather than a line of coordinates, although 420′ may also include optimal stimulation parameters 420. Paresthesia threshold pth may also vary within a range, and as noted earlier can be useful to include in the optimal stimulation parameters 420′, because pth may be helpful to permitting the patient to vary stimulation from sub-perception to supra-perception, as discussed in some later examples.

FIGS. 20C and 20D depict optimal parameters 420′ in graphical form, showing at each frequency a statistically-relevant range of pulse widths, and a statistically-relevant range of amplitudes appropriate for the patient. While optimal parameters 420′ in this example comprise a three-dimensional volume of coordinates (F, PW, and A), they are depicted in two two-dimensional graphs for easier illustration, similar to what occurred earlier in FIGS. 18E and 18F: FIG. 20C shows the relationship between frequency and pulse width, and FIG. 20D shows the relationship between frequency and amplitude. Note also that FIG. 20D shows the paresthesia threshold pth, which like pulse width and amplitude can statistically vary within a range. Optimal stimulation parameters 420 (determined without statistical variance, see FIGS. 18E and 18F) are also shown for each of the parameters, and as expected fall within the broader volume for the parameters specified by 420′.

With a volume of optimal parameters 420′ defined, it may then be useful to allow the patient to use his external controller 45 to navigate different setting within this volume of optimal parameters 420′. This is shown in one example in FIG. 20E. Here, the GUI of the external controller 45 displays not a single linear slider, but a three-dimensional volume representative of the volume 420′ of optimal parameters, with different axes representing changes the patient can make in frequency, pulse width, and amplitude. As before, the GUI of the external controller 45 allows the patient some flexibility to modify stimulation parameters for his IPG or ETS, and allows the patient to adjust all three stimulation parameters simultaneously through one adjustment action and using a single user interface element.

Different GUIs to allow the patient to navigate through the determined volume of optimal parameters 420′ are possible, and FIG. 20F shows another example. In FIG. 20F, two sliders are shown. The first, a linear slider controlled by cursor 430a, allows the patient to adjust the frequency in accordance with frequencies reflected in the optimal volume 420′. A second two-dimensional slider controlled by cursor 430b allows the patient to adjust pulse width and amplitude at that frequency. Preferably, the range of pulse widths and amplitudes is constrained by the optimal parameters 420′ and by the frequency already selected using cursor 430a. For example, if the user selected to use frequency F=400 Hz, the external controller 45 can consult optimal parameters 420′ to automatically determine an optimal range of pulse widths (e.g., 175 to 210 microseconds) and amplitudes (3.7 to 4.1 mA) for the patient to use at that frequency. When the patient changes the frequency using cursor 430a, the range of permissible pulse widths and amplitudes selectable using cursor 430b can automatically change to ensure that sub-threshold stimulation remains within the volume 420′ determined to be statistically useful for the patient. In another modification not shown, the optimal stimulation parameters can be selected using a mean charge per second (MCS) model (e.g., 380, 381), as described earlier with respect to FIGS. 14A-14F. The GUI can then also allow the user to navigate within a MCS model to select optimal stimulation parameters.

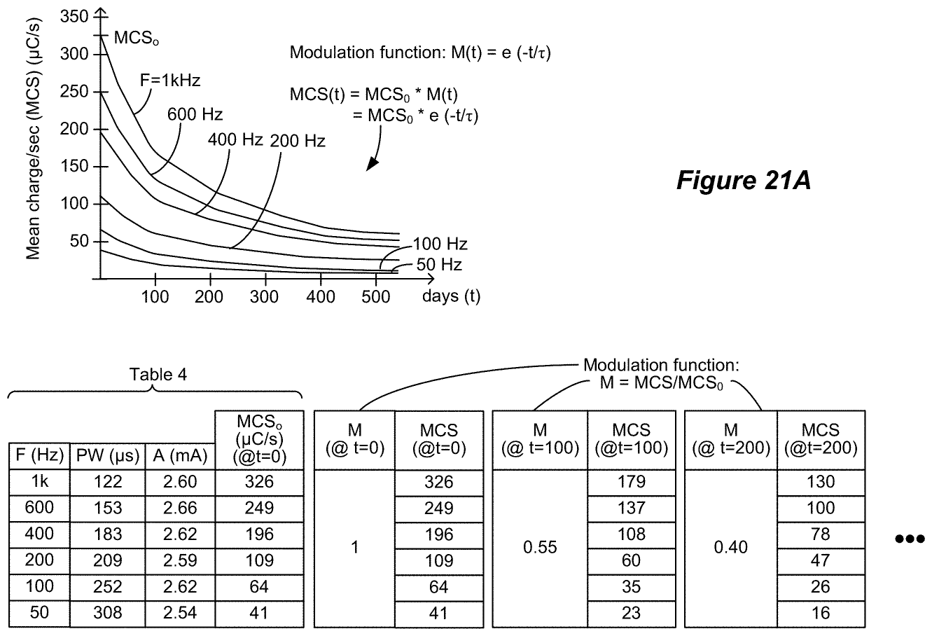

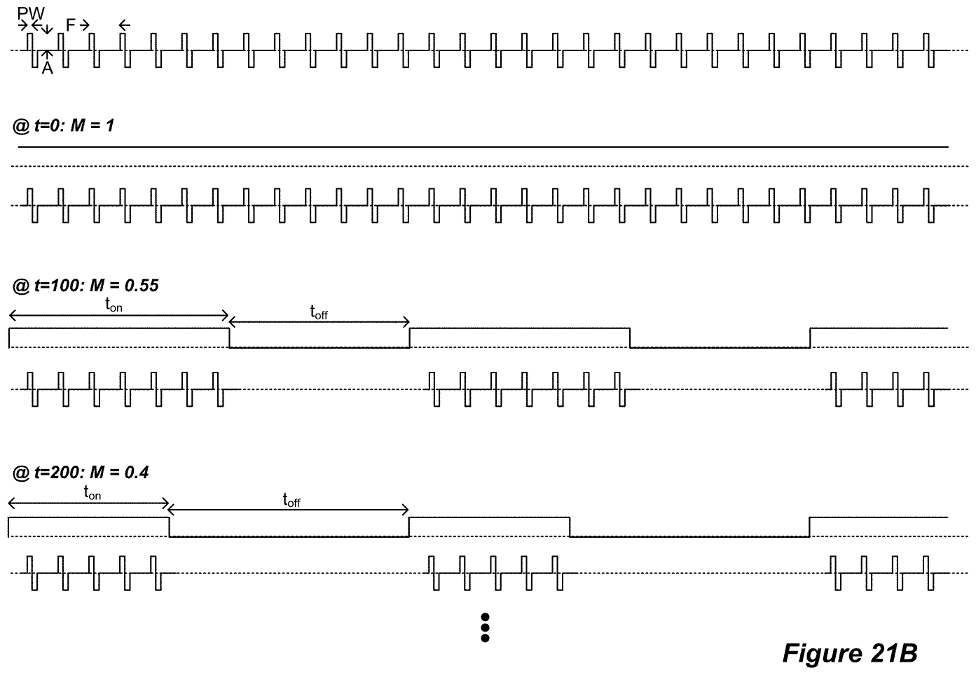

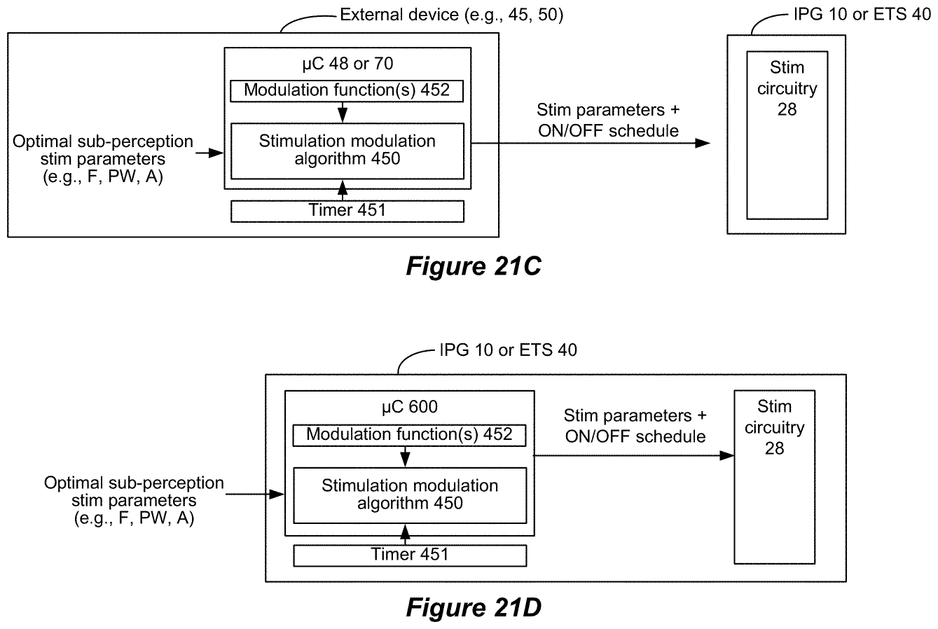

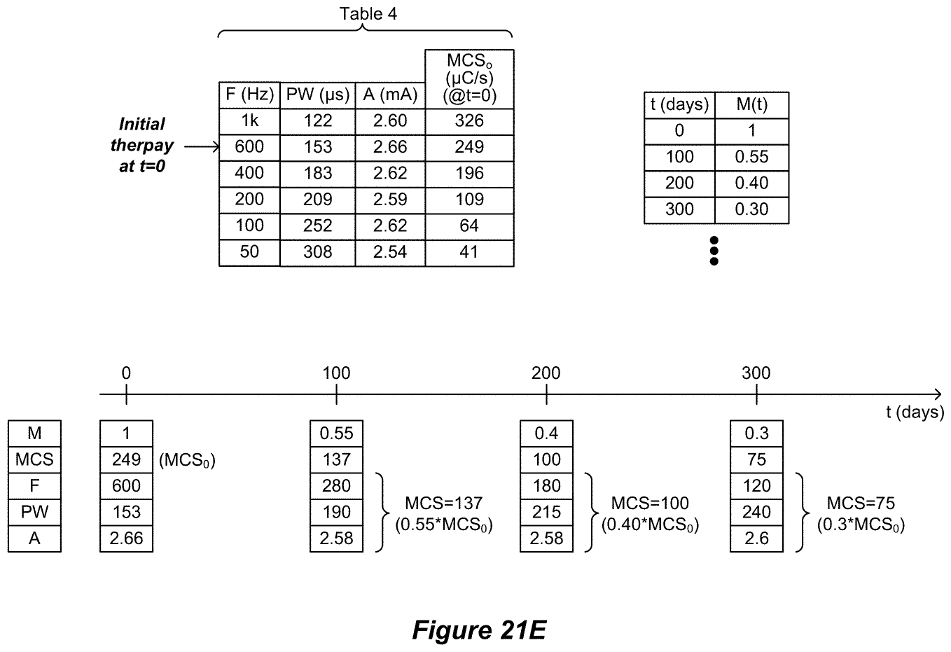

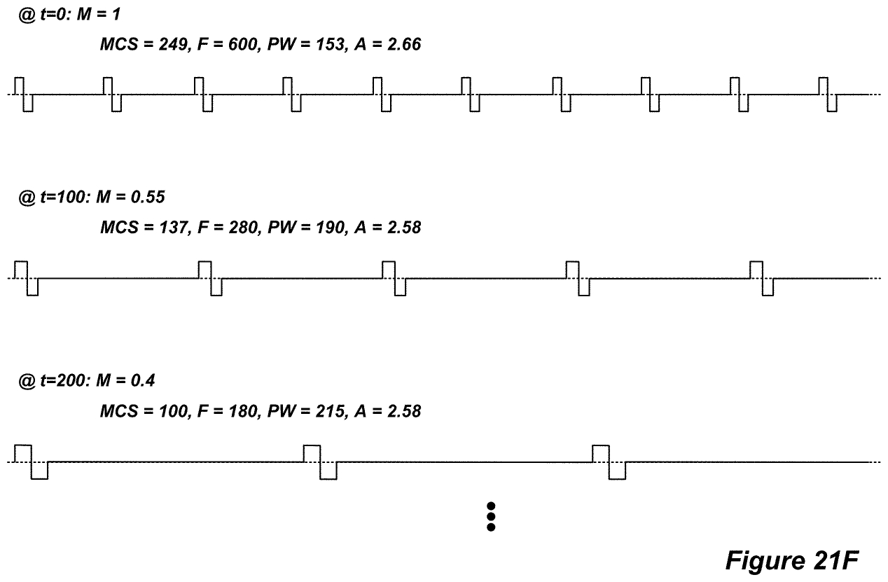

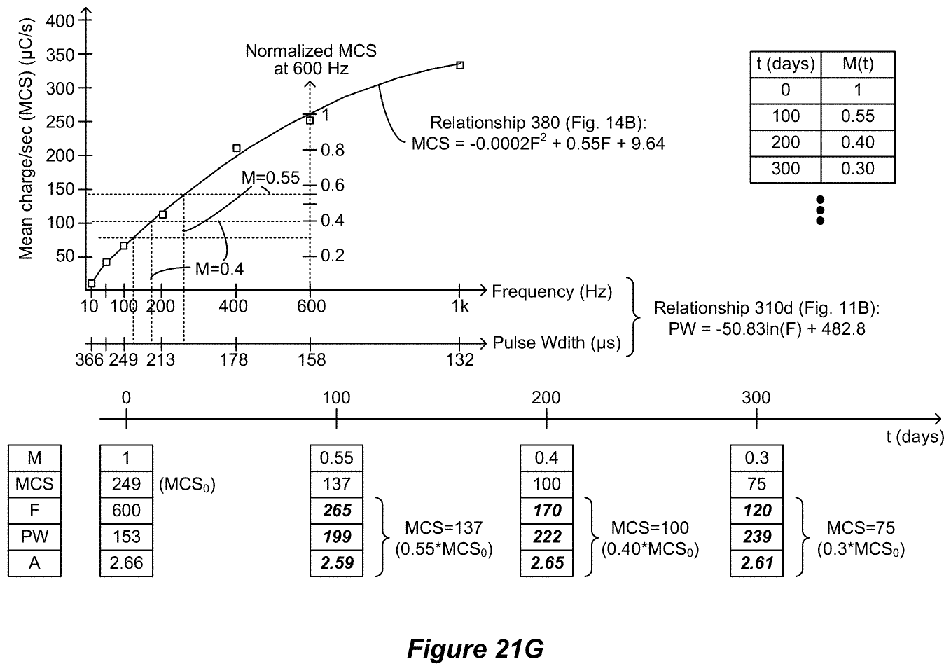

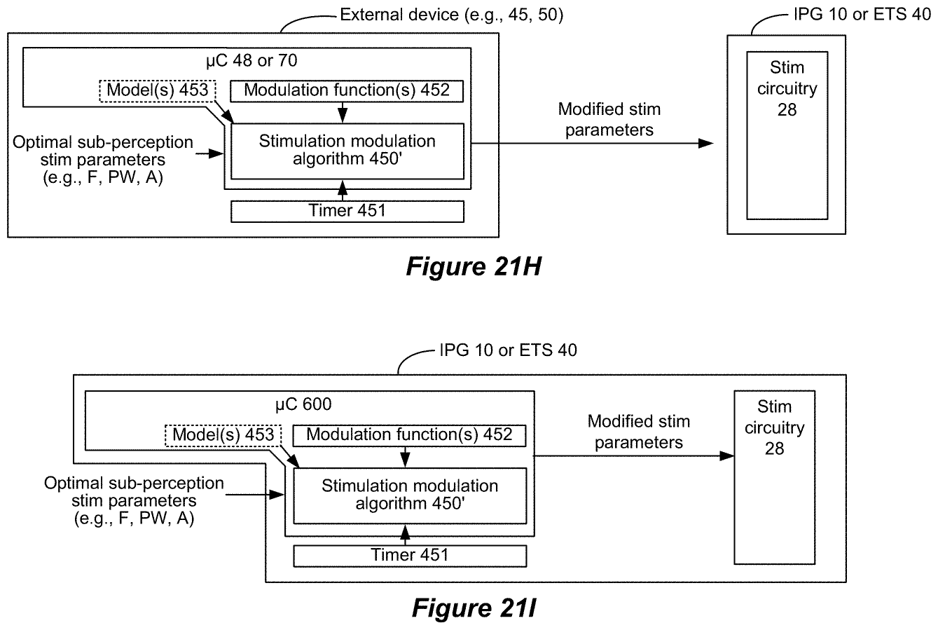

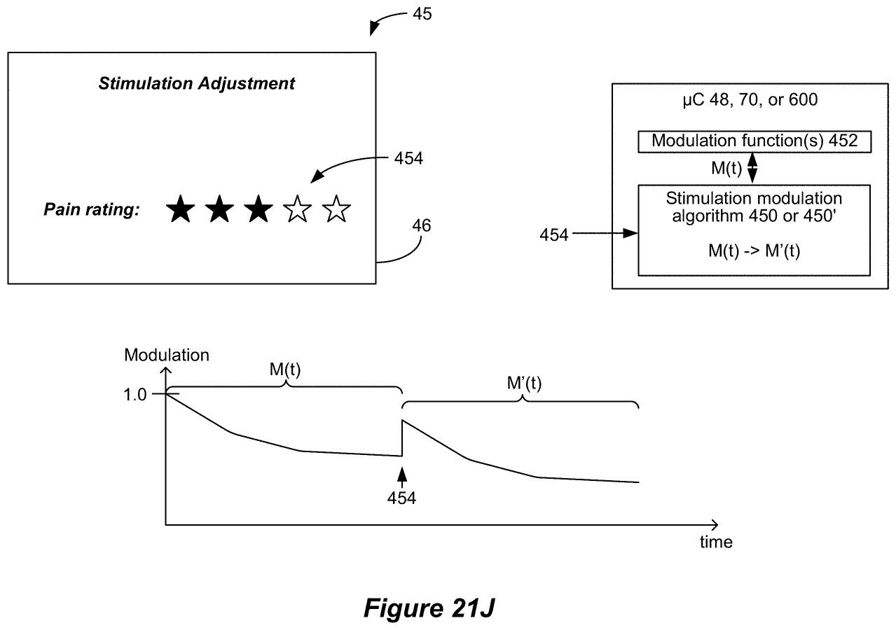

FIGS. 21A-21J illustrate other manners in which optimal sub-perception parameters determined in various manners in this disclosure can be applied to a patient. In these examples, optimal sub-perception stimulation parameters once determined for a patient are not constantly applied, and instead are subject to a modulation function M(t) which will change the amount of charge the therapy produces over time. This can be particularly useful in reducing the amount of charge the patient receives over time. In this regard, it is hypothesized that sub-perception stimulation may have a curative effect, meaning that less stimulation (less charge) is required over time. As explained below, modulating the charge that the therapy provides may involve duty cycling the optimal sub-perception stimulation, and/or may involve adjusting one or more of the parameters of that stimulation. Although illustrated as particularly useful to modulate the charge provided by sub-perception stimulation, the disclosed modulation techniques can be applied to any type of stimulation, including supra-perception stimulation.

FIG. 21A shows a first example of a modulation function M(t) that can be used to adjust the Mean Charge per Second (MCS) that sub-perception stimulation therapy delivers over time. The example modulation function comprises an exponential decay (M=e^{(-t/τ)}) which can be multiplied by a nominal MCS₀ value determined for the therapy, thus causing the MCS to decrease over time, i.e., MCS(t)=M(t)*MCS₀=MCS₀*e^{(-t/τ)}. Modeling modulation function M as an exponential decay is merely one example. Further experience in treating patients may show that other modulation functions (a linear decrease, etc.) will provide good clinician outcomes. Further, and although not shown, the modulation functions preferably do not eventually decrease the MCS to zero: that is, the MCS may approach an asymptote or a minimal value to ensure that the patient will always receive an adequate amount of charge over time. Further, a modulation function does not need to comprise an actual mathematical function, and can instead simply comprise a data structure (e.g., a table) which indicates how the MCS of the sub-perception therapy will be reduced over time (by 90% at time t1; 80% at time t2, etc.) Modulation functions M(t) may also cause the charge deliver by the therapy to increase over time, or to increase and decrease the charge over time (e.g., in a cyclic fashion), although these details aren't shown.

The nominal MCS values MCS₀ used in the modulation functions may be determined based on optimal sub-perception stimulation parameters determined generally (e.g., based on a population of patients), or as determined for a given patient, as discussed in other parts of this disclosure. For example, FIG. 21A summarizes data from table 4 discussed earlier, which shows optimal sub-perception stimulation parameters of frequency (F), pulse width (PW), and amplitude (A). From these parameters, a nominal Mean Charge per Second value (MCS₀) can be determined (e.g., MCS₀=F*PW*A) useful for the patient at time t=0. A modulation function M(t) can be applied to adjust the charge that these stimulation parameters will provide over time (MCS(t)). In the simple example depicted, the modulation function M(t) decreases the MCS by 55% at time=100 days, and by 40% at time=40 days. The table at the bottom of FIG. 21A show the effect of the modulation function M on the MCS(t) of the stimulation therapy. For example, at a frequency of 1000 Hz, the MCS delivered by the therapy starts at 326 μC/s, but drops to 179 at t=100 days, and to 130 at t=200 days. In the example of FIG. 21A, it is assumed that all of the optimal stimulation parameters are subject to the same modulation function M(t). However, the modulation function may also different for different sets of optimal stimulation parameters. For example, stimulation at 1000 Hz may use a first stimulation function, while stimulation at 600 Hz may use a second modulation function, etc.

FIG. 21B shows a first manner in which a modulation function can be applied to optimal stimulation parameters F, PW, and A. In this example, the modulation function is applied as a duty cycle to turn stimulation on and off for appropriate periods of time (tₒₙ and t_{off}) to affect a reduction in charge as a function of time. The first row of pulses in FIG. 21B shows the optimal stimulation pulses, and next rows show the effect of applying a modulation value of 1 at time t=0. As shown, the pulses remain unchanged.

The next row shows the effect of applying a modulation value of 0.55 at time t=100 days, which causes pulses to be issued for only 55% of the time (during tₒₙ). During the remaining 45% of the time, pulses are not issue (during t_{off}). These on and off periods can be interleaved and periodically applied as shown. In one example, tₒₙ might be 55 minutes, while t_{off} is 45 minutes, but this is just one example that affects a duty cycle of 55%. Notice that during the on periods, the stimulation is issued in pulses as initially determined, with F, PW, and A unchanged from their otherwise optimal values. As noted earlier, sub-perception therapy is noted to continue to be effective for some period of time after such therapy ceases, and is effective during a “wash out” period after the cessation of therapy. In this regard, stimulation during the on periods is expected to provide symptomatic relief during subsequent off periods, especially if the duration t_{off} of the off periods is equal to the wash out period. The last row in FIG. 21B shows the effect of applying a modulation value of 0.4 at time t=200 days, which causes pulses to be issued for only 40% of the time. Accordingly, tₒₙ is shorter, and t_{off} is longer, when compared to the preceding row. In another example, as M decreases, tₒₙ can be kept constant and t_{off} can be increased, or t_{off} can be kept constant and tₒₙ can be decreased. Notice that the modulation function M(t) thus scales (decreases) the charge delivered to the patient over time.

FIGS. 21C and 21D show different examples of how a modulation function M(t) can be applied to optimal sub-perception stimulation 420′ (for example, as derived in any of the manners provided in this disclosure) in the disclosed system. In either example, one or more modulation functions M(t) are stored in memory 452, and a stimulation modulation algorithm 450 is provided, which adjusts stimulation, with the one or more modulations functions to modulate the charge over time that therapy provides.

FIG. 21C shows an implementation in which the stimulation modulation algorithm 450 operates in an external device such as external controller 45 or clinician programmer 50, and more specifically operates as programmed as firmware in control circuitry 48 or 70 of those devices. The stimulation modulation algorithm 450 receives the optimal stimulation parameters 420′, and processes them using the one or more modulation functions M from memory 452 within or associated with the control circuitry 48 or 70. Timer circuitry 451 may assist the stimulation modulation algorithm 450 by providing a time basis, which can inform as to when the algorithm 450 may need to change stimulation according to the modulation function M(t) (e.g., from 1 to 0.55, or to 0.4, etc.). The stimulation modulation algorithm 450 may then determine on and off times for the stimulation using the modulation function, and in particular may provide an on/off schedule to the IPG 10 or ETS 40 along with the optimal stimulation parameters. Alternatively, the external device 45 or 50 may transmit stimulation on and off instructions to the IPG 10 or ETS 40 at the appropriate times, instead of in advance per a pre-determined schedule. Such stimulation scheduling, or receipt of stimulation on and off instructions, will allow the IPG 10 or ETS 40 to know when to activate the stimulation circuitry 28 in the IPG 10 or ETS 40 to produce pulses, hence providing to the patient an appropriate amount of charge over time.

FIG. 21D shows an implementation of the stimulation modulation algorithm 450 in an IPG 10 or ETS 40. Here, the IPG 10 or ETS 40's control circuitry 600 is programmed with the stimulation modulation algorithm 450, and with the necessary one or more modulation functions (452), which may have been programmed earlier using the external device. The IPG 10 or ETS 40 may then receive the optimal stimulation parameters 420′ parameters from the external device, and send the stimulation parameters along with any on/off instructions to the stimulation circuitry 28. Thus, in the example of FIG. 21D, the external device need not prepare stimulation scheduling for the IPG 10 or ETS 40, because such scheduling is determined by the stimulation modulation algorithm 450 in the IPG 10 or ETS 40 using the modulation function(s) 452.

FIGS. 21E and 21F show another example in which a modulation function M(t) can be applied to modify the charge provided by optimal sub-perception stimulation parameters. In this example, the stimulation parameters themselves (e.g., one or more of F, PW, and A) are changed over time to achieve the charge adjustment specified by the modulation function. In this example, it is assumed that the initially-determined optimal stimulation parameters for the patient are those taken from Table 4 at F=600 Hz, and PW=153 μs, and A=2.66 mA, which yields a nominal Mean Charge per Second value MCS₀ of 249 μC/s. The modulation function M(t) to be applied to this stimulation is shown at the upper right, which as before decreases the charge delivered to the patient to 55% at time t=100 days; to 40% at time t=200 days; etc.

In this example, the simulation is not duty cycled (e.g., FIG. 21B) to achieve the charge decrease dictated by the modulation function M(t). Instead, one or more of the stimulation parameters themselves are changed. Thus, and again assuming a nominal MCS₀=249 μC/s, the MCS as dictated by the modulation function at time t=100 should be 137 μC/s (i.e., 137=0.55*249); at time t=200 should be 100 μC/s (i.e., 100=0.40*249); etc. One or more of the stimulation parameters is then adjusted to achieve the specified MCS values at the appropriate times. Thus, at time t=100 days, the frequency is adjusted to 280 Hz, the PW to 190 μs, and the amplitude A to 2.58, which yields the desired MCS value of 137 at this time (i.e., 137 μC/s=280 Hz*190 μs*2.58 mA). At time t=100 days, the frequency is adjusted to 180 Hz, the PW to 215 μs, and the amplitude A to 2.58, which yields the desired MCS value of 100 at this time (i.e., 100 μC/s=180 Hz*215 μs*2.58 mA); etc. FIG. 21E shows the effect of the application of this modulation function M(t), and when the stimulation parameters are varied per the modulation function M(t) in this manner. Notice that the stimulation pulses are not duty cycled on and off but instead run freely while achieving the appropriate amount of charge per the modulation function. That being said, both techniques—duty cycling and adjustment of the stimulation parameters—can be used together to adjust the charge of the stimulation as specified by the modulation function, although this isn't shown for simplicity.

In FIGS. 21E and 21F, one or more of the stimulation parameters are adjusted in any desired fashion to achieve, at the appropriate times, the desired charge modulation as set by the modulation function M(t). However, and as shown in FIG. 21G, the stimulation parameters can also be adjusted in accordance with other models which relates the stimulation parameters to total Mean Charge per Second values (such as relationship 380), and/or which relate the stimulation parameters to each other (such as relationship 310d). FIG. 21G shows a graph of MCS versus frequency, which was described earlier with respect to FIG. 14B, and which shows the MCS of the optimal sub-threshold stimulation parameters of table 4. As discussed earlier, the relationship of MCS to frequency can be modelled as relationship 380. It was also discussed earlier, the optimal sub-perception stimulation parameters such as frequency and pulse width may relate to each other, as reflected in relationship 310d, as described and graphed earlier with respect to FIG. 11B for the frequency range in question.

These models 380 and 310d can be used to determine how to adjust the sub-stimulation parameters in accordance with the modulation function, M(t). Assume again for example that the initially-determined optimal stimulation parameters for the patient are those taken from Table 4 at F=600 Hz, and PW=153 μs, and A=2.66 mA, which yields a nominal Mean Charge per Second value MCS₀ of 249 μC/s. This charge value at this frequency can be normalized to 1 as shown in the graph in FIG. 21G. When it is desired to change the stimulation per the modulation, relationship 380 can be used to determine a new frequency for the stimulation, with then relationship 310d used to determine a new pulse width. For example, at M=0.55 (time=100), it is seen using relationship 380 that a frequency of 265 Hz can be selected. From this frequency value, and using relationship 310d, a pulse width value of 199 μs can be selected. Because the MCS at t=100 should equal 55% of MCS₀ (i.e., MCS=137=MCS₀*0.55), an amplitude A can be determined that provides this MCS value at the selected (i.e., A=265 Hz*199 μs/137 μC/s=2.59 mA). Later, at M=0.40 (time=200), it is seen that a frequency of 170 Hz (relationship 380) and a pulse width of 222 μs can be selected (relationship 310d), with the amplitude selected (A=2.65 mA) to arrive at the desired MCS value (of 100=249*0.4). In effect then, the modulation function M(t) is applied to select stimulation parameters (in particular F and PW) that lie on relationship 310d (FIG. 11B), with the amplitude adjusted as necessary to achieve the proper amount of charge at that time. This is beneficial because, as noted earlier, relationship 310d provides information relating frequencies and pulse widths that have been noted to provide optimal sub-perception in patients. Thus, as the modulation function M(t) operates, the adjusted stimulation parameters are still those noted by earlier modelling to be optimal. This is in contrast to FIG. 21E, where the stimulation parameters were adjusted over time per the modulation function M(t) to achieve the desired charge, but not necessarily in a manner that yields optimal parameters as verified through other models.

FIGS. 21H and 21I show different manners in which optimal stimulation parameters can be determined per a modulation function as described in FIGS. 21E and 21G. FIG. 21H shows an implementation in which a stimulation modulation algorithm 450′ operates in an external device such as external controller 45 or clinician programmer 50, which is similar to what was described earlier with respect to FIG. 21C. FIG. 21I shows an implementation of the stimulation modulation algorithm 450′ in an IPG 10 or ETS 40, similar to what was described earlier with respect to FIG. 21D. In either case, the stimulation modulation algorithm 450′ in this circumstance determines how to adjust the initially-provided stimulation parameters (F, PW, A) per a modulation function (452) to achieve an appropriate level of charge over time. As noted earlier with respect to FIG. 21G, one or more models 453 (such as relationships 380 and 310d) may be stored in conjunction with the control circuitry and used as explained earlier to determine stimulation parameter adjustment. Model(s) 453 may also not be necessary or used when the stimulation modulation algorithm adjusts the stimulation parameters, as shown for example with respect to FIG. 21E.

FIG. 21J shows that an initially selected modulation function can be varied based on objective or subjective measurements. In this regard, a modulation function may from time to time require adjustment. For example, if the modulation function decreases the charge delivered to the patient too quickly, the patient may not receive an adequate amount of charge to address their symptoms. If a patient's symptoms are returning, the patient may use the GUI of his external controller 45 to rate or rank his symptom (pain), as shown by input 454. Input 454, a subjective measurement based on the patient's assessment, may be received by the stimulation modulation algorithm 450 or 450′, which can assess the measurement and decide per its programming whether the modulation function needs to be adjusted.

In this example, upon receipt of measurement 454, the algorithm 450 or 450′ decides to increase the modulation function to provide more charge to the patient. This increase in modulation is represented by a new modulation function M′(t), which can comprise an adjustment of the original modulation function chosen (M(t)), or which may comprise another modulation function that the algorithm 450 or 450′ selects from memory 452. An adjusted modulation function M′(t) may also be stored back into memory 452 by the algorithm 450 or 450′, and the algorithm may also store in the memory 452 a history of modulation that it has used to treat the patient. The historical modulation function used may be of interest for the patient or clinician to read out of memory 452 and review in the future to get a sense of how the algorithm 450 or 450′ has adjusted the patient's charge and over therapy.

Other objective measurements can be used as input 454 to the algorithm 450 or 450′ to adjust the modulation function, or to select use of a new modulation function. For example, and as described in U.S. Patent Application Publication 2019/0209844, the IPG may detect neural responses to the stimulation, such as Evoked Compound Action Potentials (ECAPs). Various features of the sensed ECAPs can provide an indication of the effectiveness of the stimulation, and thus can be used in whole or part to adjust the modulation function. Still other subjective and objective measurements of stimulation therapy effectiveness can be used as input 454 to the algorithm 450 or 450′, some of which are discussed elsewhere in this disclosure.

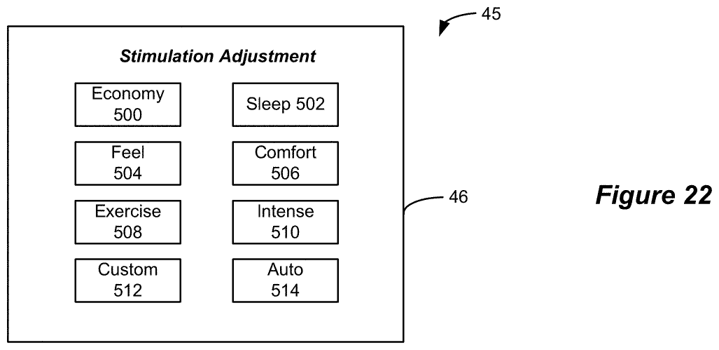

FIG. 22 shows another example in which a user can program settings for his IPG 10 (or ETS) using the derived optimal stimulation parameters. Subsequent examples for completeness use determined volumes 420′ of optimal stimulation parameters, but vectors or ranges 420 of optimal stimulation parameters could be used as well. Optimal stimulation parameters can also be selected using a mean charge per second (MCS) model (e.g., 380, 381), as described earlier with respect to FIGS. 14A-14F.

FIG. 22 shows a user interface on screen 46 of the patient's external controller 45, which allows the patent to select from a number of stimulation modes. Such stimulation modes can include various ways in which the IPG can be programmed consistent with optimal stimulation parameters 420′ determined for the patient, such as: an economy mode 500 that provides stimulation parameters having a low power draw; a sleep mode 502 which optimizes the stimulation parameters for the patient while sleeping; a feel mode 504 which allows a patient to feel the stimulation (supra-perception); a comfort mode 506 for normal everyday use; an exercise mode 508 that provide stimulation parameters appropriate for when the patient is exercising; and an intense mode 510 usable for example if the patient is experiencing pain, and would benefit from more intense stimulation. Such stimulation modes can be indicative of a patient's posture or activity. For example, a sleep mode 502 provides stimulation optimized for sleep (e.g., when the patient is lying down and is not moving significantly), and an exercise mode 508 provides stimulation optimized for exercise (e.g., when the patient is standing up and is moving significantly). Although not shown, stimulation modes can also be included that provide stimulation optimized for different patient postures, such as supine, prone, standing, sitting, etc., or for different conditions such as cold or bad weather. While illustrated in the context of the patient's external controller, realize as in other examples that another external device usable to program a patient's IPG can be used as well to select the stimulation modes, such as the clinician programmer 50.

A patient can select from these stimulation modes, and such selections can program the IPG 10 to provide a subset of stimulation parameters useful for that mode governed by the optimal stimulation parameters 420′. For some stimulation modes, the subset of stimulation parameters may be wholly constrained by (wholly within) the volume of optimal stimulation parameters 420′ determined for the patient, and hence would provide optimal sub-perception stimulation therapy for the patient. The subsets for other modes may only be partially constrained by the optimal stimulation parameters, as explained further below. In all cases however, the subsets are determined using the optimal stimulation parameters (either 420 or 420′). Preferably, the subsets are determined for the patient at the clinician programmer 50 and are transmitted to and stored in the patient's external controller 45. Alternatively, the determined optimal stimulation parameters can be transmitted to the external controller 45, leaving it to the external controller 45 to determine the subsets from the optimal stimulation parameters.

The number of stimulation modes made available for selection by the patient on the external controller 45 may be limited or programmed by a clinician. This may be warranted because some stimulation modes may not be relevant for certain patients. In this regard, the clinician may program the patient's external controller 45 to specify the stimulation modes available, such as by entering an appropriate clinician's password. Alternatively, the clinician may program the external controller 45 using clinician programmer 50 to program the external controller 45.

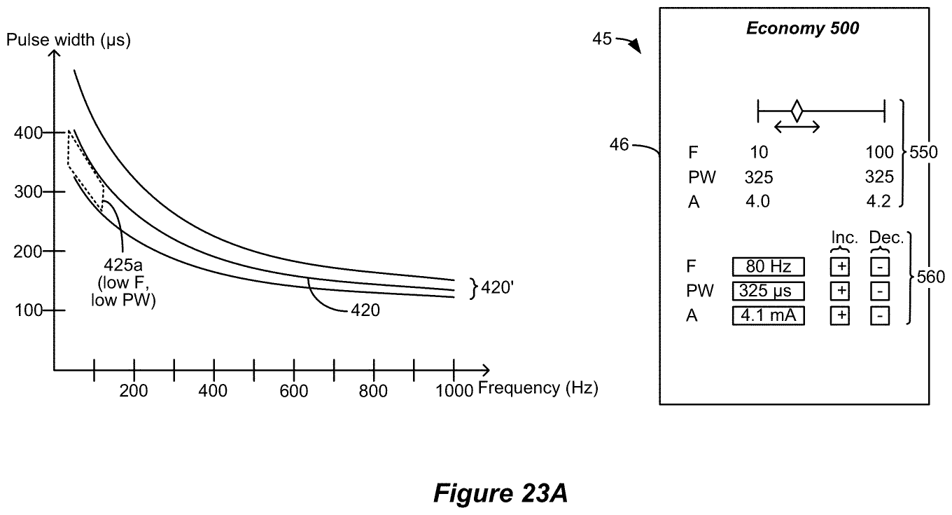

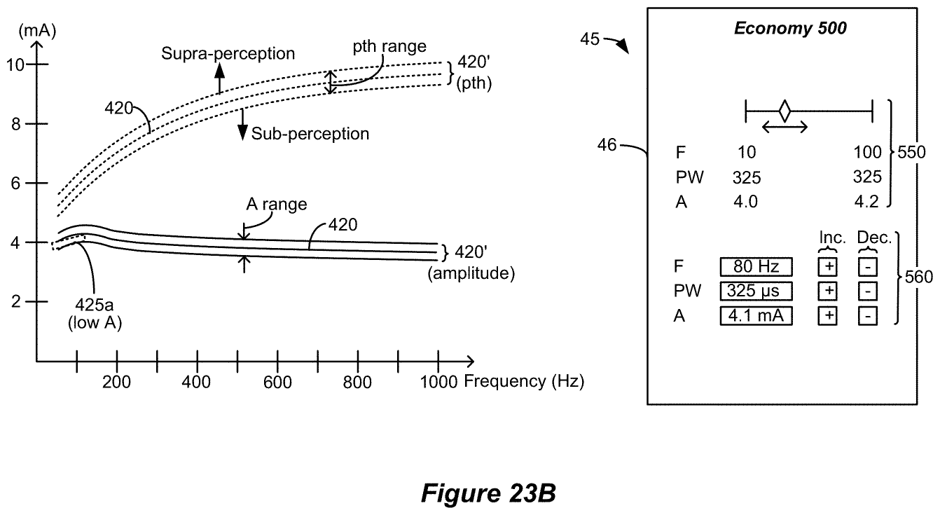

Examples of the subsets 425x of stimulation parameters are shown in FIGS. 23A-28B. FIGS. 23A and 23B show a subset 425a of stimulation parameters coordinates used when the economy mode 500 is selected, which comprises a subset of the optimal stimulation parameters 420′ having a low power draw. Subset 425a may, like optimal stimulation parameters 420′, comprise a three dimensional volume of F, PW, and A parameters, and again (compare FIGS. 20C and 20D), two two-dimensional graphs are used to represent subset 425a, with FIG. 23A showing the relationship between frequency and pulse width, and with FIG. 23B showing the relationship between frequency and amplitude.

To affect a low power draw, frequencies within subset 425a are low, such as limited to a frequency range of 10 to 100 Hz, even though the optimal stimulation parameters 420′ may have been determined over a wider range, such as 10 to 1000 Hz. Further, while optimal pulse widths within this frequency range may vary more significantly in optimal stimulation parameters 420′, subset 425a may be constrained to lower of these pulse widths, such as the lower half of such pulse widths, as shown in FIG. 23A. Again, using a lower pulse width will result in lower power draws. Furthermore, subset 425a may be constrained to lower amplitudes within optimal stimulation parameters 420′ for the relevant frequency range, as shown in FIG. 23B, again resulting in lower power draws. In short, subset 425a can comprise a smaller volume of stimulation parameters wholly within the volume of optimal stimulation parameters 420′ that provide adequate sub-threshold stimulation for the patient, while providing lower power draws from the IPG's battery 14. Not all subsets 425x corresponding to selected stimulation modes (FIG. 22) contain stimulation parameters that are necessarily wholly within the determined optimal stimulation parameters 420′ as shown in some subsequent examples.

When economy mode 500 is selected, the external controller 45 could simply transmit a single low-power optimal parameter (F, PW, A) within subset 425a to the IPG for execution. However, and more preferably, the user interface will include means to allow the patient to adjust stimulation parameters to those within subset 425a. In this regard, the user interface can include a slider interface 550 and a parameter interface 560. The slider interface 550 can be as explained earlier (see FIG. 19), and can include a cursor to allow the patient to slide through parameters in subset 425a. In the example shown, slider interface 550 may not adjust the pulse width, which is set to a particular value (e.g., 325 μs), but the frequency and amplitude can vary. This is just one example, and all three of frequency, pulse, and amplitude may be variable by the slider in other examples, or other of the parameters may be held constant. Note that more complicated user interfaces can be used to allow the patient to navigate through subset 425a. For example, although not shown, user interface elements having a more three-dimensional quality, such as those discussed earlier in FIGS. 20E and 20F, can be used to navigate the volume of subset 425a. Parameter interface 560 can also allow the patient to navigate parameters within subset 425a, and is shown simply as having selectable buttons to increase or decrease the parameters within the determined subset 425a. Parameter interface 560 may also include fields displaying the current values for frequency, pulse width, and amplitude. Initially, these values may be populated with parameters that are roughly in the center of the determined subset 425a, thus allowing the patient to adjust the stimulation around that center.

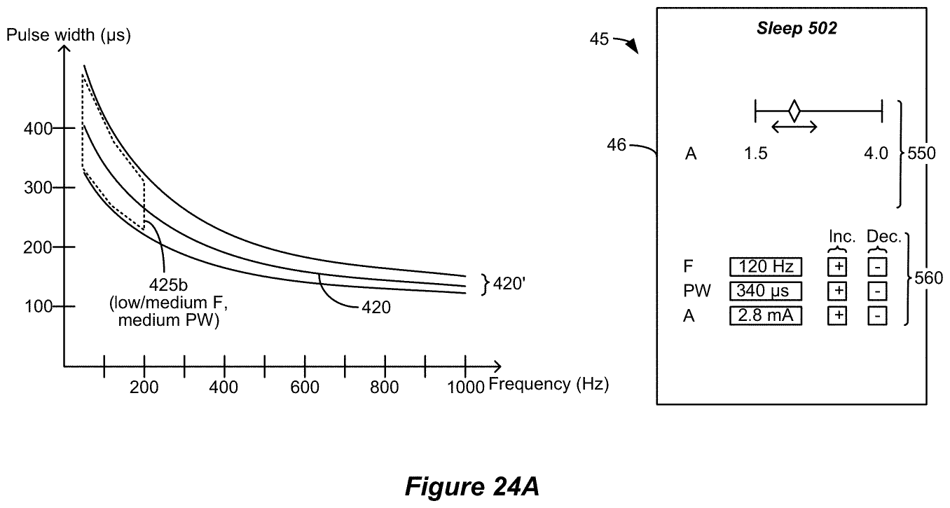

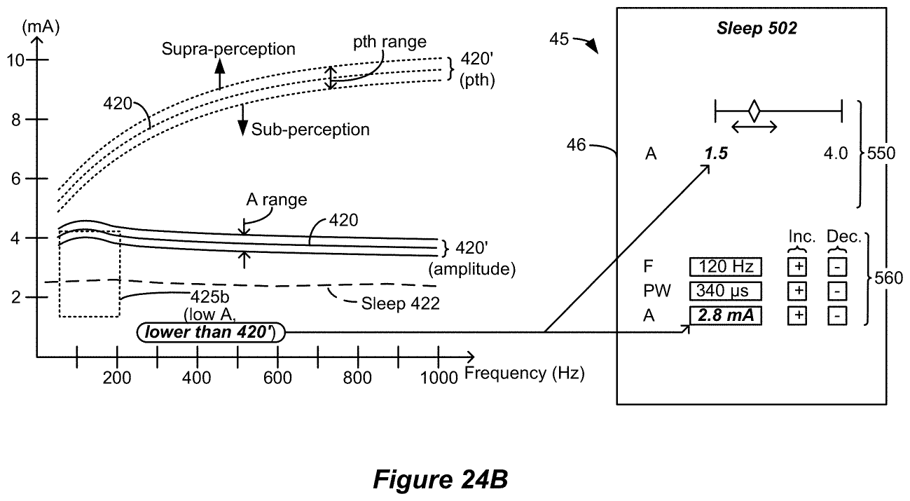

FIGS. 24A and 24B shows selection of sleep mode 502, and the subset 425b of optimal stimulation parameters 420′ that results when this selection is chosen. Subset 425b in this example is determined using the optimal stimulation parameters 420′ in a manner such that subset 425b is only partially constrained by optimal stimulation parameters 420′. Subset 425b may include low-to-medium frequencies (e.g., 40 to 200 Hz) within optimal stimulation parameters 420′, and can include medium pulse widths otherwise permitted by 420′ for this frequency range, as shown by FIG. 24A.

Because the intensity of the stimulation may not need to be as high during sleep, amplitudes within subset 425b may fall outside of amplitudes otherwise suggested by optimal parameters 420′, as shown in FIG. 24B. For example, while optimal parameters 420′ may suggest for example that the amplitude based on earlier modelling would fall within a range of 3.6 to 4.0 mA for the frequency and pulse width ranges of interest, the amplitude within subset 425b in this example be set to even lower values. Specifically, as shown in the slider interface 550, the amplitude can be set between 1.5 mA and 4.0 mA. To know where the lower boundary of amplitude should be set, modeling information can include an additional model 422, which may be determined separately from optimal stimulation parameters 420′ based on patient testing. Permitting the use of amplitudes lower than those suggested by optimal parameters 420′ may be warranted in the case of sleep due to expected changes in the location of the electrodes leads within a patient's spinal column when the patient is lying down. Further, a patient may be less bothered by pain while sleeping, and therefore lower amplitudes could still be reasonably effective. This being said, subset 425b could also comprise values (including amplitude) wholly within and constrained by optimal stimulation parameters 420′, similar to what was shown for subset 425a in FIGS. 23A and 23B.

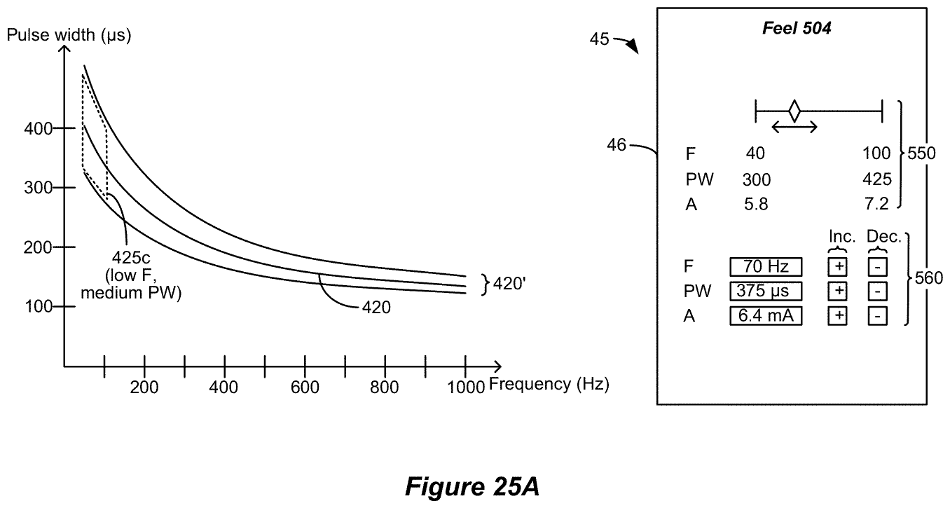

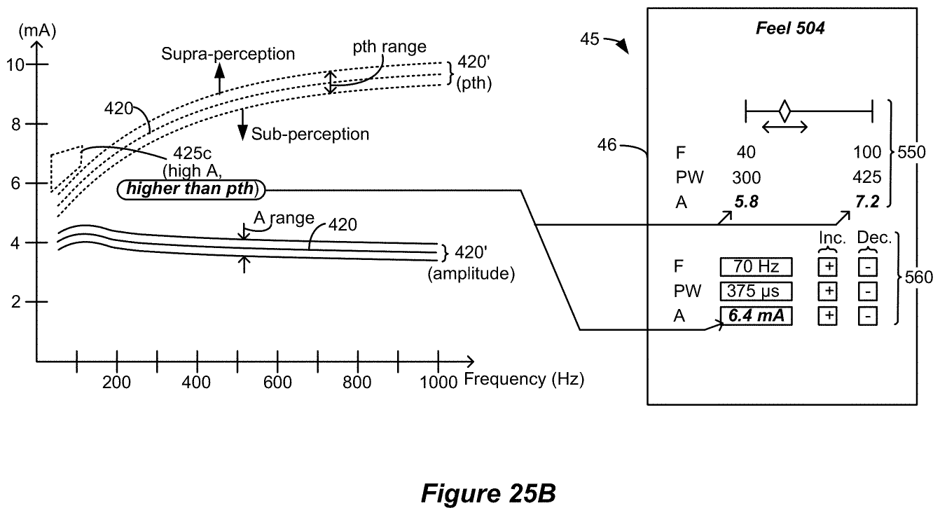

FIGS. 25A and 25B show selection of feel mode 504, and the resulting subset 425c useable for a given patient during this mode. The purpose of this mode is to allow the patient, at their discretion, to feel the stimulation that his IPG is providing. In other words, the stimulation provided to the patient in this mode is supra-perception. The optimal stimulation parameters 420′ preferably define a volume of stimulation parameters in which sub-perception stimulation is optimized for the patient. However, as described earlier, perception threshold pth is measured and modelled as part of the determination of optimal sub-threshold stimulation parameters 420′. As such, perception threshold pth as determined earlier is useful during this mode to select amplitudes that a patients will feel—i.e., amplitudes that are higher than pth for the other stimulation parameters (particularly pulse width). The feel mode 504 is thus an example in which it is beneficial to include pth values (or pth ranges) within optimal stimulation parameters 420′.

It is generally easier for a patient to feel stimulation at lower frequencies, and thus selection of feel mode may constrain stimulation in subset 425c to lower frequencies (e.g., 40 to 100 Hz), as shown in FIG. 25A. Control of the pulse width may not be a primary concern, and thus the pulse width may have a medium range as permitted by 420′ for this frequency range, again as shown by FIG. 25A.

However, because the patient in this mode intends to feel the stimulation, the amplitude within subset 425c is set to higher values, as shown in FIG. 25B. Specifically, the amplitudes for the relevant frequencies and pulse widths are set not only to be higher than the upper bound for amplitudes as determined for optimal stimulation parameters 420′; they are also set at or higher than the perception threshold, pth. As noted earlier, the perception threshold, pth, and more particularly significant ranges for pth as determined for the patient (when statistical variation is considered), can be included with the optimal stimulation parameters 420′ (see FIG. 20D) to useful effect in this mode. Thus, subset 425c is be defined to set the amplitude at a value or within a range that should provide supra-perception stimulation based on earlier measurements and modelling. If pth is defined by a range in light of statistical variance, the permissible range of amplitude for the feel mode 504 may be set beyond the upper value of that range, as shown in FIG. 25B. Therefore, while the optimal (sub-perception) amplitude (per 420′) for the frequency range of interest may range from about 3.7 to 4.5 mA, the amplitudes within subset 425c are set to about 5.8 to 7.2 mA, beyond the upper bound of the pth range to guarantee that the resulting stimulation is supra-perception for the patient in question. In this example, note that subset 425c is determined using the optimal stimulation parameters 420′, but is only partially constrained by such optimal parameters. Frequency and pulse width are constrained; amplitude is not, because the amplitude in this subset 425c is set beyond 420′, and more particularly beyond pth.

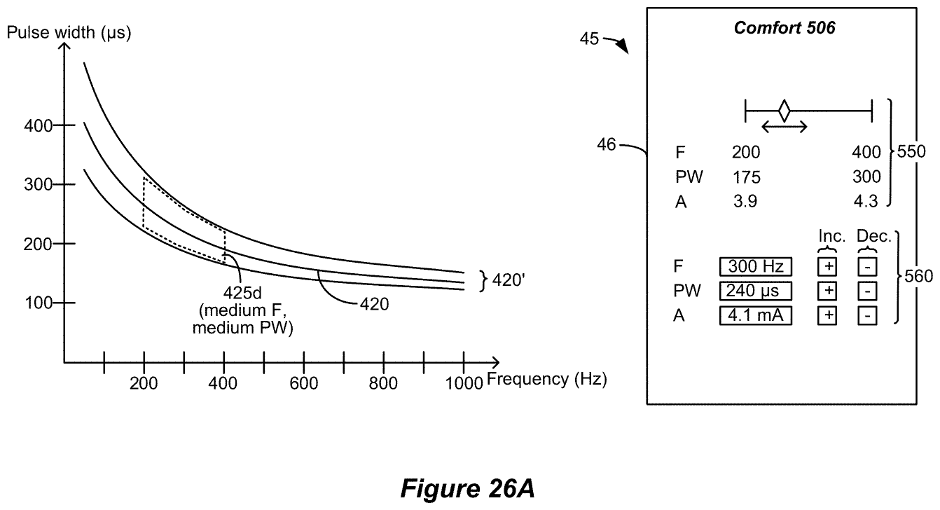

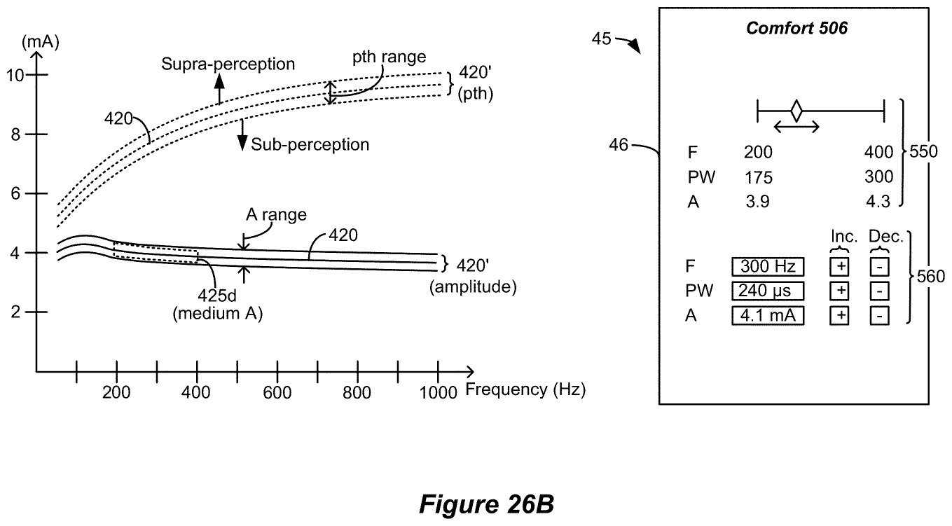

FIGS. 26A and 26B shows selection of a comfort mode 506, and the resulting subset 425d of stimulation parameters for this mode. In this mode, stimulation parameters are set via subset 425d to nominal values within the optimal stimulation parameters 420′: medium frequencies such as 200 to 400 Hz, and medium pulse widths for those frequencies, such as 175 to 300 μs as shown in slider interface 550, as shown in FIG. 26A. Amplitudes within subset 425d may likewise be medium amplitudes within optimal stimulation parameters 420′ for the frequencies and pulse widths at issue, as shown in FIG. 26B. In this example, the stimulation parameters in subset 425d are wholly constrained by optimal stimulation parameters 420′, although as noted earlier, this doesn't have to be the case for every subset.

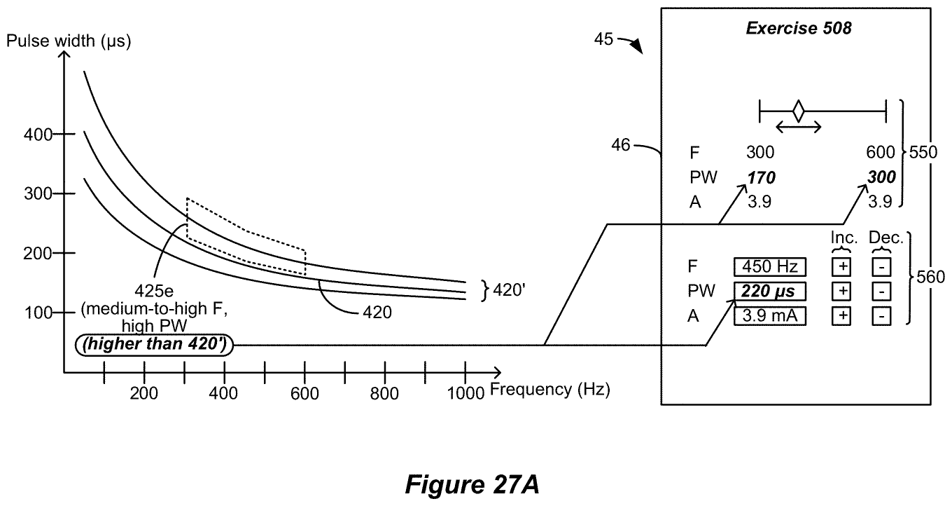

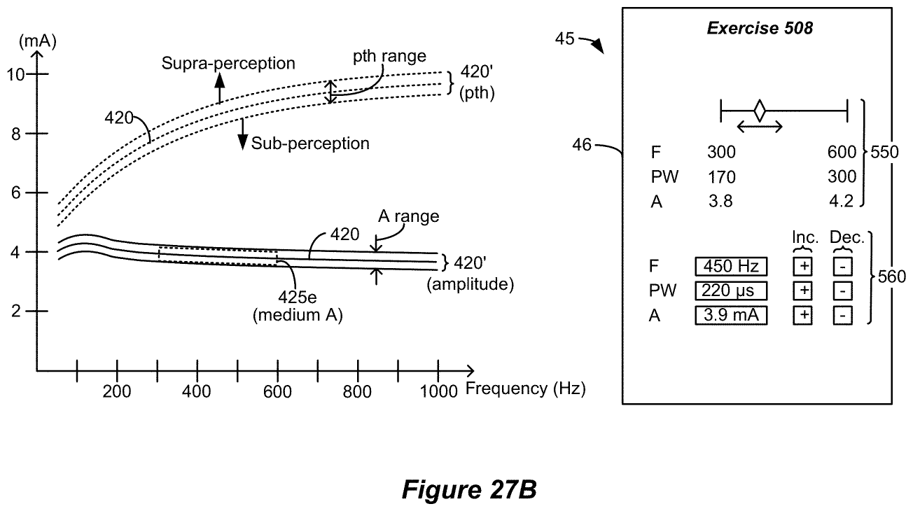

FIGS. 27A and 27B show selection of an exercise mode 508, and the subset 425e of stimulation parameters associated with this mode. In this mode, it may be warranted to use medium-to-high frequencies (e.g., 300-600 Hz), but pulse widths that are higher than those prescribed by optimal stimulation parameters 420′ for these frequencies, as shown in FIG. 27A. This is because the position of the electrode leads in the patient may be more variable when the patient is moving, and hence it may be useful to provide higher injections of charge into the patient which higher pulse widths would achieve. As shown in FIG. 27B, the amplitudes used may span a medium range for the frequencies and pulse widths involved, but higher amplitudes beyond 420′ (not shown) could also be used to provide additional charge injection as well. Subset 425e shows an example where the frequency and amplitude are constrained by optimal stimulation parameters 420′, but pulse width in not; thus subset 425e is only partially constrained by optimal stimulation parameters 420′. Subset 425e in other examples could be wholly constrained within the optimal stimulation parameters 420′ determined earlier.

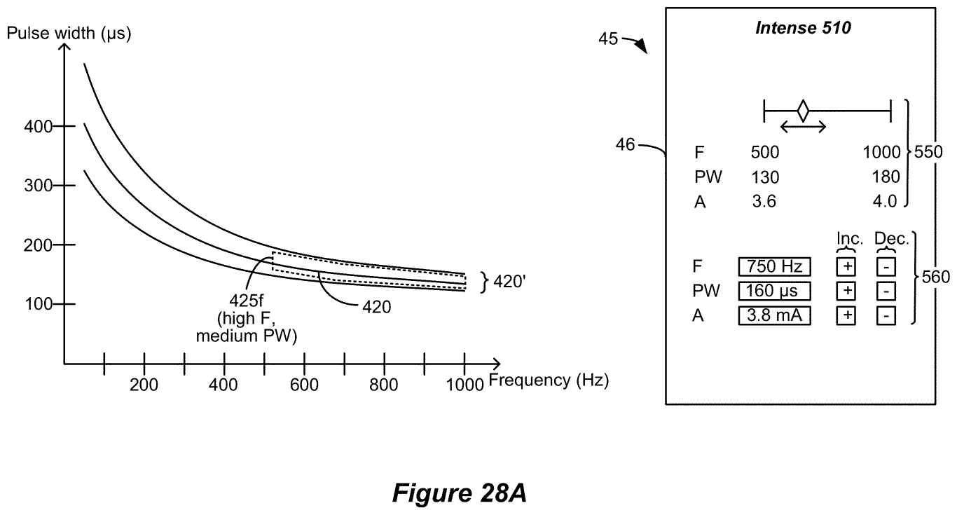

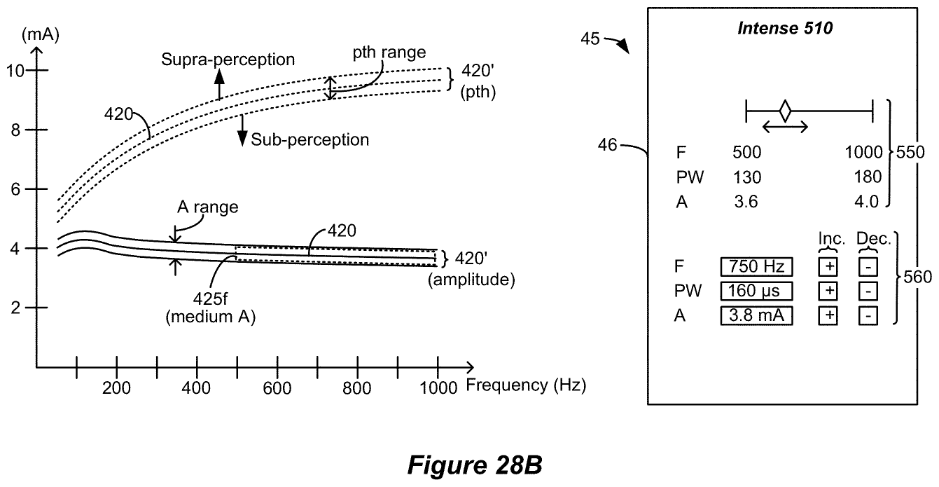

FIGS. 28A and 28B show selection of an intense mode 510 of stimulation. In this mode, stimulation is more aggressive, and the subset 425f of stimulation parameters may occur at higher frequencies (e.g., 500 to 1000 Hz). However, the pulse width and amplitudes at these frequencies may be medium for the frequencies involved, as shown in FIGS. 28A and 28B respectively. In this example, the subset of stimulation parameters in subset 425f may be wholly constrained by (contained within) optimal stimulation parameters 420′. As in earlier examples, the patient can use interfaces 550 or 560, or other interface elements not shown, to adjust stimulation within the subset 425x corresponding to the patient's stimulation mode selection (FIG. 22). Less preferably, selection of a stimulation mode may cause the external controller 45 to send a single set of stimulation parameters (F, PW, A) determined using the optimal stimulation parameters 420′ (or 420).

Notice that the stimulation parameters in subsets 425x may overlap; some F, PW, and A values in one subset (e.g., 425a) may also be present in another subset (e.g., 425b). In other words, it is not strictly necessary that stimulation parameters in a given subset are unique to that subset, or the stimulation mode that that subset represents, although this could also be the case. Furthermore, the boundaries of the various subsets 425x may be adjustable. For example, although not shown, the external controller 45 could have options to change the boundaries for the various subsets. Using such options, a patient or clinician could for example change one or more of the stimulation parameters (e.g., frequency) in a subset (e.g., by increasing the frequencies within subset 425a from 10 to 100 Hz to 10 to 150 Hz). Adjustments to the subsets 425x may also be affected in response to certain feedback, such as patient pain ratings as may be entered into the external device 45, or detection of patient activity or posture. More complex adjustments may be locked to the patient, and only made accessible by the clinician, with such accessibility being provided by entering a password into the external controller 45 for example. Behind such password protection, the subsets 425x may be adjustable, and/or other stimulation modes (e.g., beyond those shown in FIG. 22) may be made accessible to the clinician only. As before, clinician adjustments of this sort may also be made by the clinician using clinician programmer 50.

The subsets 425x may also be automatically updated from time to time. This may be advantageous, because the underlying modelling leading to the generation of optimal stimulation parameters 420′ may change or become better informed as data is taken on more patients. It may also later be learned that different stimulation parameters better produce the effects desired for the stimulation modes, and so it may be warranted to adjust which parameters are included in the subsets. Different stimulation modes, provided for different reasons or to produce different effects, may also become apparent later, and so such new modes and their corresponding subsets may be later programmed into the external controller 45, and presented to the patient in the stimulation mode user interface of FIG. 22. Updating of the subsets and/or stimulation modes can occur wirelessly, either by connection of the external controller 45 to a clinician's programmer, or to a network such as the Internet. It should be understood that the stimulation modes disclosed, and the subset of stimulation parameters 425x corresponding to such modes, are merely exemplary, and that different modes or subsets could be used.

Referring again to FIG. 22, the stimulation mode user interface can include an option 512 to allow the patient or clinician to define a custom mode of stimulation. This custom mode 512 may allow the user to select a frequency, pulse width, and amplitude, or to define a subset, at least partially defined by optimal stimulation parameters 420′. Selection of this option may provide a user interface that allows a patient to navigate different stimulation parameters within optimal stimulation parameters 420′, such as those shown earlier in FIGS. 20E and 20F. Should the patient find stimulation parameters through this option that seem effective to operate as a simulation mode, the user interface can allow the stimulation mode to be stored for future use. For example, and referring to FIG. 20E, the patient may have found stimulation parameters within optimal stimulation parameters 420′ that are beneficial when the patient is walking. Such parameters may then be saved by the patient, and appropriately labeled, as shown at user interface element 580 in FIG. 20D. This newly-saved stimulation mode may then be presented to the patient (FIG. 22) as a selectable stimulation mode. The logic in the external controller 45 may additionally define a subset 425 (e.g., 425g) of stimulation parameters through which the patient can navigate when this user-defined stimulation mode is later selected. Subset 425g may comprise for example stimulation parameters that bound the patient's selected parameters (e.g., +/−10% of the frequency, pulse width and amplitude selected by the patient), but which are still wholly or partially constrained by the optional stimulation parameters 420′.

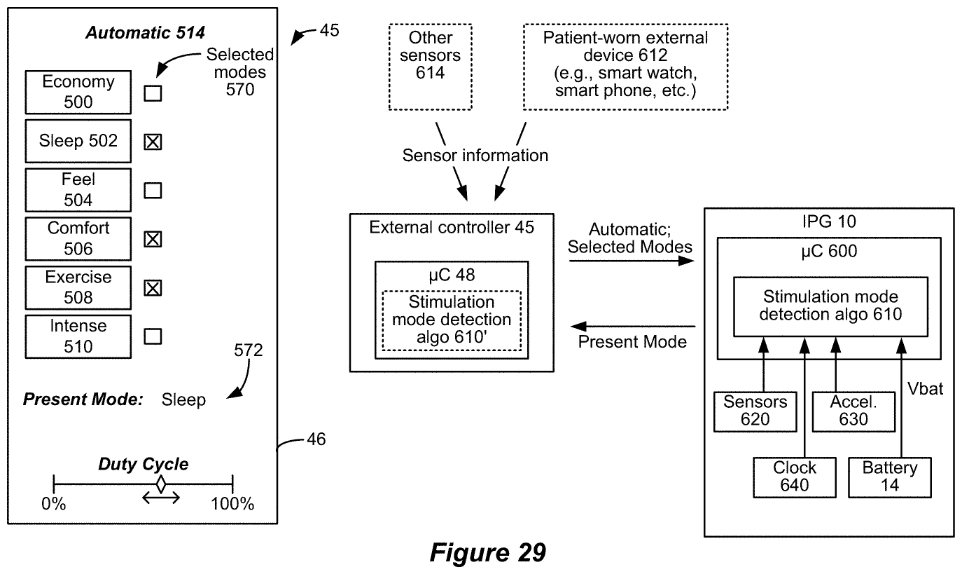

As shown in FIG. 22, the stimulation mode user interface can also include an option 514 that automatically selects and adjusts the stimulation mode for the patient based on various factors that the IPG 10 may detect. Selection of this automatic mode 514 is shown in further detail in FIG. 29. Preferably, selection of the automatic mode 514 allows the patient to select 570 which of the stimulation modes he would like detected, and to be automatically used by his IPG 10. In the depicted example, the user has selected the sleep mode 502, the comfort mode 506, and the exercise mode 508. The IPG 10 will try to automatically detect when these stimulation modes should be entered, and in this regard the IPG 10 can include a stimulation mode detection algorithm 610. As shown, this algorithm may be programmed into the control circuitry 600 of the IPG 10. The control circuitry can comprise a microprocessor, microcomputer, an FPGA, other digital logic structures, etc., which is capable of executing instructions an electronic device. Alternatively, algorithm 610 in the IPG 10 can attempt to detect, and adjust stimulation for, all stimulation modes (e.g., 500-510) supported by the system, without the need for the user to select 570 stimulation modes of interest.

Algorithm 610 can receive different inputs relevant to detecting the stimulation mode, and hence subsets 425x, that should be used for a patient at any given time. For example, the algorithm 610 may receive input from various sensors that indicate the posture and/or activity level of the patient, such as an accelerometer 630. The algorithm 610 may also receive input from various other sensors 620. In one example, the sensors 620 can include the electrodes Ex of the IPG 10, which can sense various signals relevant to stimulation mode determination. For example, and as discussed in U.S. Pat. No. 9,446,243, signals sensed at the electrodes can be used to determine (complex) impedances between various pairs of the electrodes, which can be correlated in the algorithm 610 to various impedance signatures indicative of patient posture or activity. Signals sensed at the electrodes may comprise those resulting from stimulation, such as Evoked Compound Action Potentials (ECAPs). Review of various features of detected ECAPs can be used to determine patient posture or activity, as disclosed in U.S. Patent Application Publication 2019/0209844. Signals sensed at the electrodes may also comprise stimulation artifacts resulting from the stimulations, which can also indicate patient posture or activity, as disclosed in U.S. Patent Application Publication 2020/0251899. Sensed signals at the electrodes can also be used to determine a patient's heart rate, which may also correlate to patient posture or activity, as disclosed in U.S. Patent Application Publication 2019/0290900.

The algorithm 610 can receive other information relevant to determining stimulation modes. For example, clock 640 can provide time information to the algorithm 610. This can be relevant to determining, or confirming, whether the patient is involved in activities that occur during certain times of day. For example, it may be expected that the patient may be asleep during evening hours, or exercising during mornings or afternoon hours. Although not shown, the user interface may allow time ranges for expected activities to be programmed, such as whether a patient prefers to exercise in the morning or afternoon. The algorithm 610 can also receive input from the battery 14, such as the current state of the battery's voltage, Vbat, which may be provided by any number of voltage sensors, such as an Analog-to-Digital Converter (ADC; not shown). This can be useful for example in deciding when the economy mode 500 or other power-based stimulation mode should be automatically entered, i.e., if Vbat is low.

In any event, the stimulation mode detection algorithm 610 can wirelessly receive an indication that the automatic mode 514 has been selected, as well as any of the selected modes 570 of interest to the patient. The algorithm 610 can then determine using its various inputs when those modes should be entered, and thus will enable the use of the subsets 425x corresponding to the detected stimulation modes at appropriate times. In the example of FIG. 29 for example, the algorithm 610 may determine using the accelerometer 630, sensors 620, and the clock 640 that a person during evening hours is still, supine, or prone, and/or that his heart rate is slow, and thus determine that the person is presently sleeping. Algorithm 610 may at that time automatically activate sleep mode 502, and activate use of stimulation parameters within subset 425b (FIGS. 24A-24B) corresponding to this mode. Further, the IPG 10 may transmit notice of the present stimulation mode determination back to the external controller 45, which may be displayed at 572. This can be useful to allow the patient to review that the algorithm 610 has correctly determined the stimulation mode. Further, notifying the external controller 45 of the presently-determined mode can allow the proper subset 425x for that mode to be used by the external controller 45 to allow a patient adjustment to stimulation. That is, the external controller 45 can use the determined mode (sleep) to constrain adjustment (FIGS. 24A-24B) to the corresponding subset (425b) for that mode.

If the algorithm 610 determines using one or more of its inputs that a person is quickly changing position, is upright, and/or that his heart rate is high, it may determine that the person is presently exercising, a stimulation mode of interest selected by the patient. Algorithm 610 may at that time automatically activate exercise mode 508, and activate use of stimulation parameters within subset 425e (FIGS. 24A-24B) corresponding to this mode. Again, the IPG 10 may transmit notice of this present stimulation mode determination back to the external controller 45, to constrain adjustment (FIGS. 27A-27B) to the corresponding subset (425e) for that mode. If the algorithm 610 cannot determine that the patient is sleeping or exercising, it may default to a selection of the comfort mode 506, and provide stimulation, notification, and constrain adjustment (subset 425d, FIGS. 26A-26B) accordingly.

The external controller 45 may also be useful in determining the relevant stimulation mode to be used during selection of the automatic mode. In this regard, the external controller 45 can include sensors useful to determine patient activity or posture, such as an accelerometer, although this isn't shown in FIG. 29. The external controller 45 can also include a clock, and can wirelessly receive information from the IPG 10 concerning its battery voltage, and from sensors 620 regarding signals that are detected at the IPG's electrodes. Thus, the external controller 45 may also include a stimulation mode detection algorithm 610′ responsive to such inputs. This algorithm 610′ can take the place of algorithm 610 in the IPG 10, or can supplement the information determined from algorithm 610 to improve the stimulation mode determination. In short, and as facilitated by the bi-directional wireless communication between the external controller 45 and the IPG 10, the stimulation mode detection algorithm can effectively be split between the external controller and the IPG 10 in any desired fashion.

Further, the external controller 45 can receive relevant information to determine which stimulation mode should be entered from various other sensors. For example, the external controller 45 can receive information from a patient-worn external device 612, such as a smart watch or smart phone. Such smart devices 612 contain sensors indicative of movement (e.g., an accelerometer), and can include biological sensors as well (heart rate, blood pressure), which can be helpful to understanding different patient states, and thus different stimulation modes that should be used. Other sensors 614 more generically can also provide relevant information to the external controller 45. Such other sensors 614 could include other implantable devices that detect various biological states of the IPG patient (glucose, hear rate, etc.). Such other sensors 614 can provide still other information. For example, because cold or bad weather has been shown to affect an IPG patient stimulation therapy, sensor 614 could comprise weather sensors that provide weather information to the external controller 45. Note that sensor 614 may not need to communicate directly with the external controller 45. Information from such sensors 614 can be sent by a network (e.g., the Internet) and provided to the external controller 45 via various gateway devices (routers, WiFi, Bluetooth antennas, etc.).

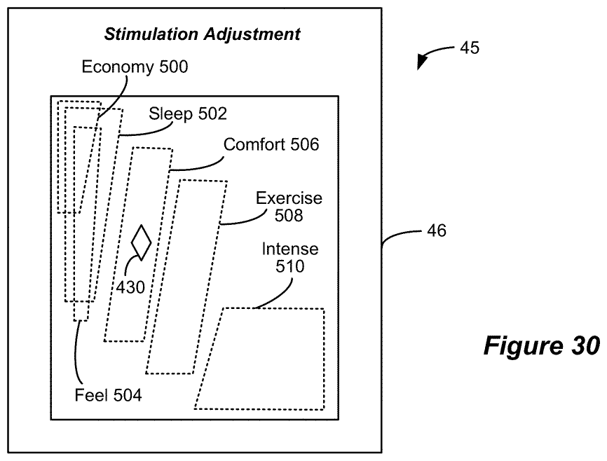

FIG. 30 shows another example of a user interface on the patient's external controller 45 that allows a patient to select from different stimulation modes. In this example, the different stimulation modes (consistent with optimal stimulation parameters 420′ determined for the patient) are displayed in a two-dimensional representation. In the example shown, the two-dimensional representation comprises a graph of pulse width (Y axis) versus frequency (X-axis), but any two stimulation parameters (amplitude versus frequency, or pulse width versus amplitude) could have been used as well. However, note that these X and Y axes may not be labeled, nor labeled with particular pulse width or frequency values, if the goal is to provide the patient with a simple user interface unencumbered by technical information that the patient may not understand.

Labeled in this two-dimensional representation are the different stimulation modes discussed earlier, with boundaries showing the extent of the subsets 425x of each stimulation mode. Using this representation, the patient can position a cursor 430 to select a particular stimulation mode, and in so doing select a frequency and pulse width, and its corresponding subset 425x. Because the subsets 425x may overlap, selection at a particular frequency and pulse width may select more than one stimulation mode, and more than one subset 425x, thus allowing the patient to navigate through more than one subset of stimulation parameters. Because amplitude is not represented in the two dimensional representation, the amplitude may automatically be adjusted to a suitable value given the stimulation mode/subset 425x, or the particular frequency/pulse width, selected. Alternatively, a separate slider can be included to allow the patient to additionally adjust the amplitude in accordance with subsets 425x for each of the stimulation modes. As explained above, the amplitude may be wholly constrained within optimal stimulation parameters 420′ by the selected mode/subset, or may be allowed to range beyond 420′ (e.g., FIGS. 24B, 25B). In a more complex example, the representation could include a three-dimensional space (F, PW, A) in which the patient can move the cursor 430, similar to that shown in FIG. 20E, with three-dimensional subsets 425x for the stimulation modes displayed.

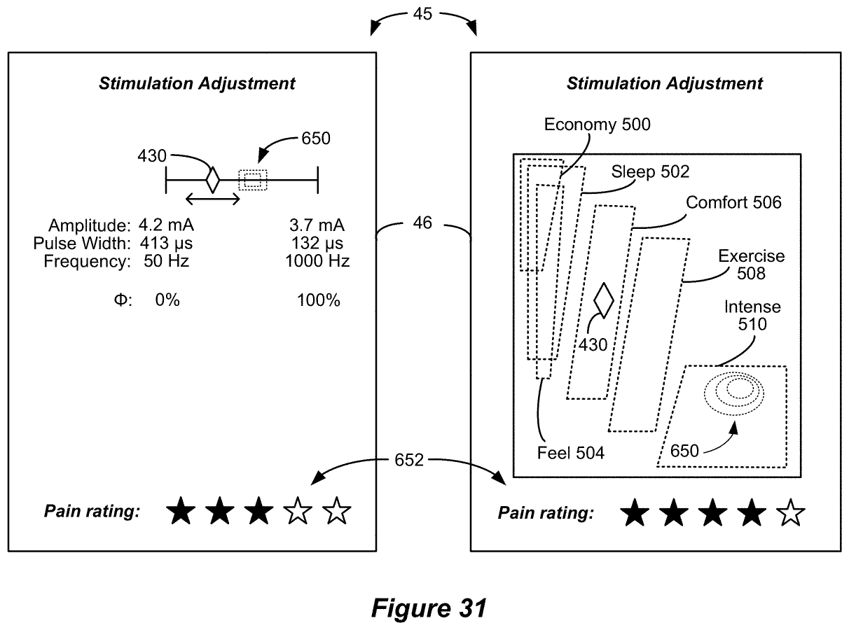

FIG. 31 shows another GUI aspect that allows a patient to adjust stimulation in accordance with the modelling developed for the patient. In these examples, a suggested stimulation region 650 is shown for the patient, overlaid on user interface elements that otherwise allow the patient to adjust stimulation. The examples in FIG. 31 show modification to the graphical user interfaces shown in FIGS. 19 and 30, but could be applied to other user interface examples as well. In these examples, suggested stimulation region 650 provides for the patient a visual indicator where the patient may want to select (using cursor 430 for example) stimulation settings consistent with optimal stimulation parameters 420 or 420′, or subsets 425x. These regions 650 can be determined in different manners. They can be mathematically determined using the optimal stimulation parameters 420 or 420′ or subsets 425x, such as by determining a center or “center of mass” of such regions. They may also be determined with specific focus on providing stimulation parameters that have an appropriate amplitude, intensity, or total charge for the patient. This may be particularly useful if the patient's previous selections have moved far away from such ideal values. Regions 650 may also be determined during a fitting procedure—by determining regions or volumes that the patient most prefers within optimal stimulation parameters 420 or 420′ or subsets 425x.

Furthermore, regions 650 can be determined over time for the patient based on previously selected stimulation parameters. Thus, regions 650 can correlate to setting most often used by the patient. In an improved example, the patient may also provide feedback relevant to determining the location of regions 650. For example, the external device 45 can include an option 652 to allow a patient to provide an indication of their symptoms (e.g., pain) using a rating scale as shown. Over time, the external controller can track and correlate the pain ratings input at 652 with the stimulation parameters selected, and draw or update region 650 to appropriate locations overlaying the stimulation adjustment aspects where the patient has experienced the best symptomatic relief. Again, a mathematical analysis weighting stimulation parameters versus their pain ratings, or a center of mass approach, can be used.

It should be noted the use of the disclosed techniques should not necessarily be limited to the specific frequencies tested. Other data suggests applicability of the disclosed technique to provide pain relief without paresthesia at frequencies as low as 2 Hz.

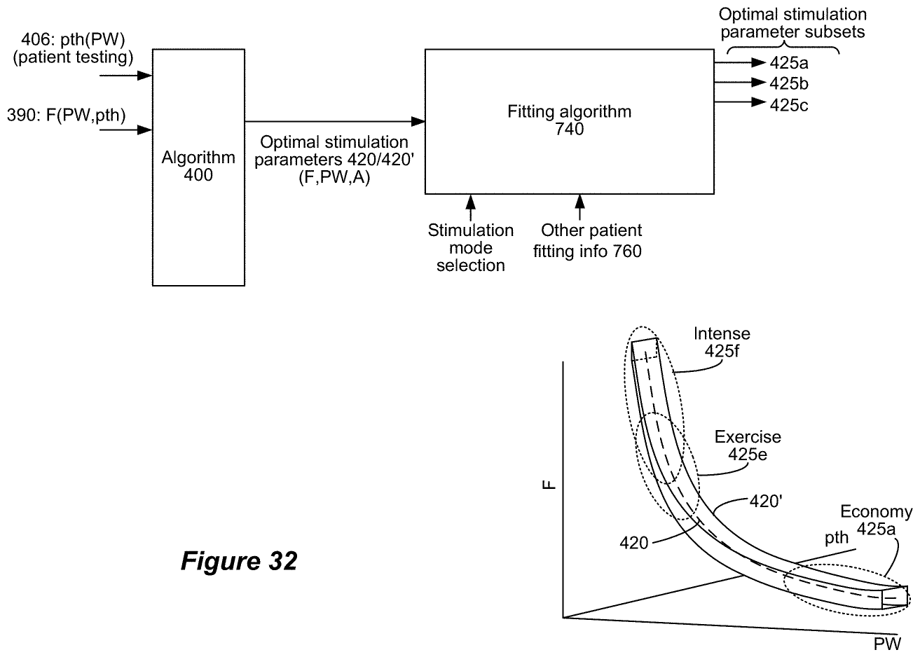

FIG. 32 summarizes aspects disclosed to this point to determine optimal stimulation parameters for the patient. As noted, such optimal parameters preferably provide optimal sub-perception stimulation therapy for the patient. As mentioned earlier, this isn't strictly necessary, because the disclosed techniques can also be used to provide supra-perception therapy as well.

FIG. 32 shows algorithm 400 as disclosed earlier (FIG. 18A et seq.), which is used to determine (preferably) optimal sub-perception parameters for the patient. As described earlier, the algorithm 400 can determine these optimal parameters 420/420′ using as inputs a model 390 (FIG. 15) modeling for example frequency as a function of pulse width and perception threshold (390), and measured parameters taken from the patient, such as for example paresthesia threshold measurements at different pulse widths (406; FIG. 18A). Further, subsets of optimal stimulation parameters 425i can be determined from the optimal stimulation parameters 420/420′ using a fitting algorithm 740. This fitting algorithm 740 can receive as an input a stimulation mode selection (FIG. 22), as described earlier in FIGS. 23A-28B. Such stimulation modes can include as examples economy (500), sleep (502), feel (504), comfort (506), exercise (508), and intense (510) stimulation modes, which can depend on activities or desires of the patient and dictate therapy accordingly. Further, fitting algorithm 740 can determine relevant subsets 425i of optimal stimulation parameters using other patient fitting information 760, as described in U.S. Patent Application Publication 2020/0147397, which is incorporated herein by reference in its entirety. As described in the '397 Publication, patient fitting information 760 can include inputs such as paresthesia overlap mapping data, perception threshold levels, questionnaire results, etc. As described earlier and shown in the graph in FIG. 32, the optimal parameters can comprise a line of stimulation parameter (e.g., F,PW,A) coordinates (420), or can comprise a statistically-significant volume of such coordinates (420′). Subsets 425i of coordinate corresponding to parameters best for particular stimulation modes are also shown in this graph.

As explained elsewhere in this application and those incorporated, the processing of information as shown in FIG. 32A to determine optimal stimulation parameters for the patient 420/420′, or subsets 425i of stimulation parameters, is beneficial for the patient, as it allows optimal stimulation to be determined with added precision and depending on particular circumstances. This takes some of the guess work out of selecting a particular stimulation parameter set (F, PW, A) for use by the patient, because the universe of stimulation parameters that the IPG is capable is producing is narrowed. This is especially helpful when sub-perception therapy is used, because the patient can't feel the stimulation directly.

However, as the graph in FIG. 32 illustrates, the optimal stimulation parameters or subsets preferably comprise a plurality of potentially-useful individual stimulation parameter sets (F,PW,A). But not all of these stimulation parameter sets may be equally useful in resolving the patient's symptoms. Therefore, the patient or clinician still must ultimately select one stimulation parameters set from this plurality at any given time for use by the patient. This may be difficult for the clinician or patient to navigate using their clinician programmer 50 or external controller 45, given the complexity which with the stimulation parameters sets were determined.

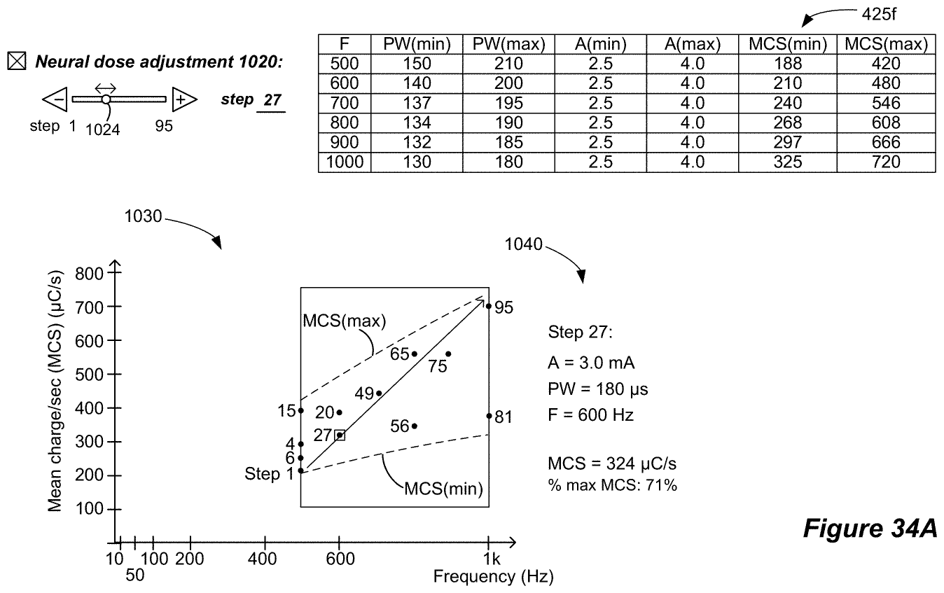

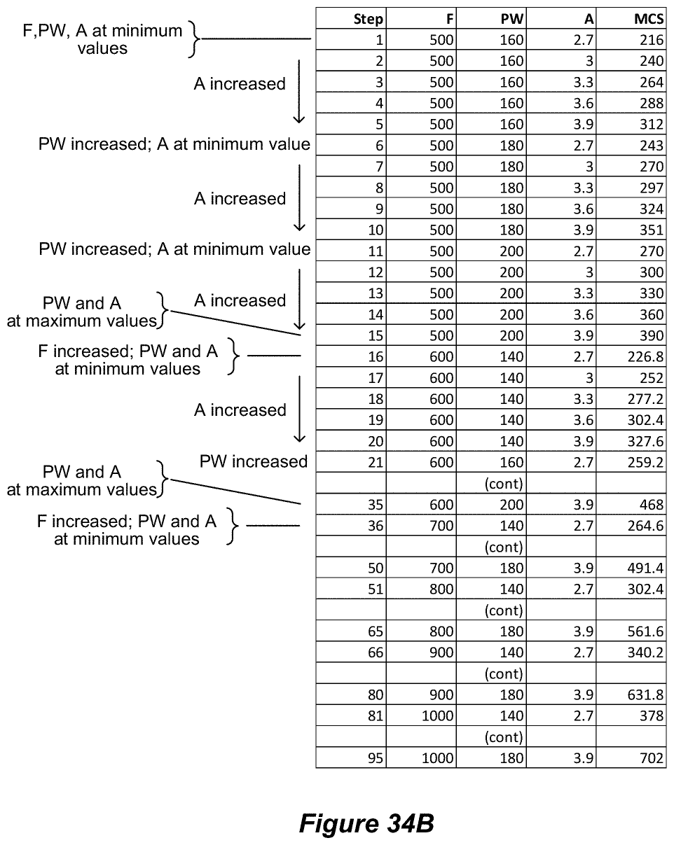

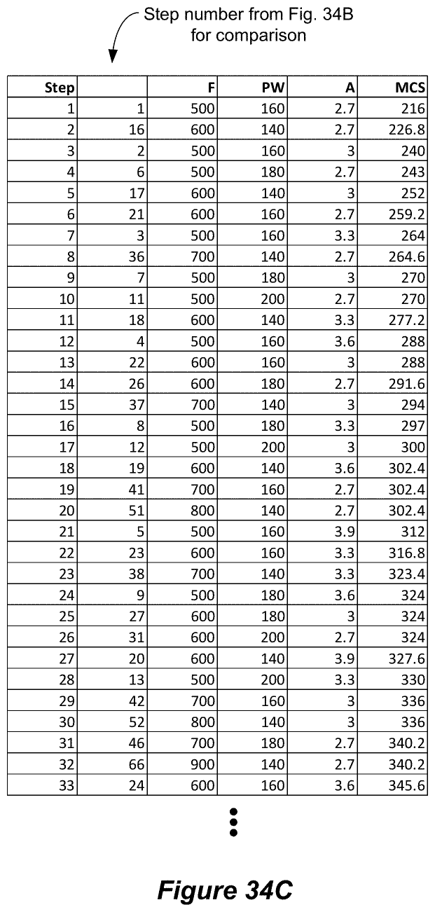

To assist in resolving this problem, FIGS. 33A-34C describe manners in which the clinician or patient can navigate the optimal stimulation parameters 420/420′ or subsets 425i to arrive at selection of a single stimulation parameter set for use by the patient at any given point in time. Specifically, and referring first to FIG. 33A, a graphical user interface GUI 1000 is shown, which may be rendered on the clinician programmer 50 or the external controller 45. It is assumed that this GUI 1000 is particularly useful after optimal stimulation parameters 420/420′ have been arrived at, as explained further below.

GUI 1000 in this example shows the leads interface 92 including the electrode array 17 and the location of the stimulation in the array. More relevantly, the GUI 1000 includes a modeling input 1002. When selected, this input 1002 allows the user to select whether sub-perception modeling is to be used in selecting stimulation parameters, or whether no modeling is used. In FIG. 33A, no modeling has been selected, which allows the patient to freely select stimulation parameters in a conventional fashion. In this regard, the GUI 1000 includes up and down arrows to allow the amplitude (A), pulse width (PW), and frequency (F) to be adjusted. The values of these parameters as selected can be displayed textually. Further, they may be represented by indicators 1004 superimposed on windows 1006. The windows 1006 may indicate a potential range for the parameter in question: in this example, amplitude A is assumed to be adjustable from 0 to 10 mA, pulse width from 0 to 500 microseconds, and frequency from 0 to 2000 Hz. However, this is just one simple example; in another example, amplitude A may be adjustable from 0 to 10 mA, pulse width from 0 to 1000 microseconds, and frequency from 2 to 1200 Hz. These ranges may be determined in accordance with the values that the IPG is able to produce. The indicators 1004 may move left or right in the windows 1006 to spatially indicate the values of the parameters within the windows' ranges. When no modeling is selected using input 1002, notice that the windows 1006 are not constrained because the user can select any value for each of the stimulation parameters.

FIG. 33A also shows battery information 1008 indicative of the performance of the IPG's battery dependent on the stimulation parameters that have been selected. As noted earlier, an IPG can have a primary (permanent) battery, or a rechargeable battery, and information 1008 can display useful information assuming either type of battery is used. For example, if the IPG has a primary battery, information 1008 may display the estimated remaining life of the battery. If the IPG has a rechargeable battery, information 1008 may display information relevant to charging, such as how frequently the IPG must be recharged and/or how long each charging session will take. In either case, such information 1008 can be estimated by GUI 1000 using the selected stimulation parameters, which generally indicate the amount of power the IPG will expend when providing stimulation. Techniques for determining information 1008 on the external device in which GUI 1000 runs can be found in U.S. Pat. No. 9,433,796, which is incorporated herein by reference in its entirety. Information 108 could also be shown in further examples of GUI 1000 that follow, but for simplicity is not shown again in subsequent figures.

FIG. 33B show selection at modeling input 1002 of sub-perception (sub-p) stimulation therapy. It is assumed in this example that when this option is selected, optimal stimulation parameters 420/420′ (determined earlier) are considered, and are used by GUI 1000 to constrain the (sub-perception) stimulation providable to the patient. Selection of this option can also cause additional aspects to populate in the GUI. For example, a stimulation mode input 1003 may be shown, which may be used to select from the stimulation modes described earlier (economy, sleep, etc.). Finally, a neural dose adjustment input 1020 can also be displayed, and is explained later.

As mentioned, stimulation parameters selectable in FIG. 33B are constrained by the optimal stimulation parameters, and in particular simulation parameters 420′ defining a volume of (F,PW,A) coordinates. An example of optimal stimulation parameters 420′ is shown in FIG. 33C. As shown, optimal stimulation parameters 420′ are indexed using frequency, with each frequency being associated with a maximum and minimum pulse width and amplitude. That being said, the volume of optimal stimulation parameters 420′ could be represented differently. Note for simplicity in FIG. 33C it is assumed that the amplitude A ranges between a minimum value of 2.5 mA and a maximum value of 4.0 mA for all frequencies, although as noted earlier the modelling and computations may provide different values for the amplitude at different frequencies. The maximum and minimum pulse widths in FIG. 33C by contrast vary between maximum and minimum values consistent with earlier modeling.

Referring again to FIG. 33B, when sub-p modeling is selected (1002), the user can select a stimulation mode (1003), or no particular mode as FIG. 33B shows. Even when a particular stimulation mode is not selected, the selection of the stimulation parameters can be constrained in the GUI 1000 using the optimal stimulation parameters 420′. Further, such constraints can be visualized for the user. In this regard, the windows 1006 for each stimulation parameter can include exclusion zones 1010 representing values for parameters that cannot be selected. Exclusion zones 1010 can be determined in accordance with the optimal stimulation parameters 420′ as shown in FIG. 33C. For example, these parameters 420′ specify that only frequencies from 100 to 1000 Hz are appropriate based on the sub-perception modelling and patient measurement take earlier. Thus, the frequency is only adjustable within an inclusion zone 1012 between and including these values. Note that indicator 1004 can still be displayed within this inclusion zone 1012 to indicate the frequency the user has selected (300 Hz in the example in FIG. 33B).

Other stimulation parameters like amplitude and pulse width can be similarly constrained. In one example, and consistent with the manner in which parameters 420′ are indexed (FIG. 33C), the ranges for the parameters in inclusion zones 1012 may dynamically depend on a value selected for one of the stimulation parameters. For example, permissible ranges for the amplitude and pulse width may depend on the frequency that has been selected. For example, and referring to FIG. 33C, at F=300 Hz the pulse width varies between 180 and 250 microseconds, and the amplitude varies between 2.5 and 4.0 mA. Exclusion and inclusion zones 1010 and 1012 are thus populated in the GUI 1000 to limit adjustment of these values given the user's selection of F=300 Hz. In sum, constraining the parameters as shown in FIG. 33B ensures that the user can select stimulation parameters for the patient that are consistent with parameters 420′ determined from modeling. Note that the GUI 1000 can also include options 1005 to lock certain of the parameters so that they cannot be automatically adjusted. This is useful to keep a given stimulation parameters constant while others may be automatically adjusted. For example, the amplitude can be locked. If the user thereafter adjusts the frequency, the pulse width value and its associated inclusion zone 1012 may be changed, but the amplitude value will not (assuming it is still within its inclusion zone 1012).

FIG. 33D shows selection of a particular stimulation mode (intense) from the stimulation mode input 1003. This constrains the selectable stimulation parameters in accordance with the subset 425f (see also FIGS. 28A and 28B). As explained earlier, subsets 425i comprise particular subsets of stimulation parameters chosen consistently with optimal stimulation parameters 420′, and generally comprise a particular subset of stimulation parameters within 420′. That being said, and as explained earlier, subsets 425i may also be determined in accordance with the parameters 420′, but may also include at some parameters sets outside of 420′. See, e.g., FIGS. 24A&B (discussing the sleep stimulation mode); 25A&B (discussing the feel stimulation mode); 27A& B (discussing the exercise stimulation mode). The relevant subset 425f for the stimulation mode selected in FIG. 33D (intense) is shown in FIG. 33E. For simplicity, it is assumed that this subset 425f comprises the same parameters 420′ shown earlier (FIG. 33C), but with the frequency range limited to 500-1000 Hz. Note that that certain stimulation modes may be recommended for a particular patient based on patient-specific metrics, such as paresthesia overlap mapping data, perception threshold levels, questionnaire results, etc., as discussed earlier. See other patient fitting info 760, FIG. 32.

As shown in FIG. 33D, GUI 1000 may similarly constrained based on the selected subset 425i similar to what was described earlier (FIGS. 33B&C). Again, exclusion and inclusions zones 1010 and 1012 can be provided in windows 1006. Thus, as shown in FIG. 2D, frequency is constrained to adjustment between 500 to 1000 Hz. Amplitude and pulse width may likewise be constrained, and as before this can occur depending on the frequency chosen. For example, at F=600 Hz, the pulse width can be constrained between 140 and 200 microseconds, and the amplitude constrained between 2.5 and 4.0 mA.

GUI 1000 as described is helpful in ultimately selecting a particular stimulation parameter set for use by the patient, because constraining selection of the stimulation parameters narrows options. Still, even with options narrowed, the GUI 1000 still ultimately relies on selection of the individual stimulation parameters. This may be difficult, in particular for the patient. If therapy is not working well for the patient, should the patient increase the amplitude or the pulse width? Or decrease these? Or change the frequency? These may be complicated questions for the uninformed patient, leading the patient to select different values at random.

This issue is addressed in GUI 1000 by the neural dose adjustment interface 1020, whose operation is explained with reference to FIGS. 34A-34C. When selected, this interface 1020 allows the user to navigate the modeled parameters 420′ (FIG. 33B) or subsets 425i (FIG. 33D). In the example of FIGS. 34A-34C, it is assumed that the intense stimulation mode has been selected (1003), and hence the user will use interface 1020 to step though stimulation parameter sets (i.e., different combinations of stimulation parameters) in subset 425f.

Interface 1020 preferably includes selectable step up and step down buttons, which increment though the stimulation parameter sets in a number of steps. In the example shown in FIGS. 34A-34C for example, it is assumed that there are 95 steps, and thus 95 stimulation parameter sets within subset 425f (FIG. 33D) that the user can cycle through. That being said, the number of steps supported by interface 1020 does not necessarily need to correspond to each and every possible stimulation parameter set within subset 425f that the IPG is able to provide, as explained further below. Furthermore, and as shown, interface 1020 can include an option 1022 to adjust the step size. This step size adjustment 1022 can control for example the amount that a given stimulation parameter is changed by interface 1020. Generally speaking, selecting a finer step size would increase the number of steps in interface 1020, while a coarser step size would decrease the number of steps. Interface 1020 can also include a slider 1024 to allow the user to slide through the different steps without using the arrows. The GUI 1000 can also provide an indication of the mean-charge-per-second (MCS) that the currently selected step is providing to the patient, to give the patient some idea whether the MSC is relatively high (e.g., near 100%) or low. This percentage may be determined relative to a maximum MCS (MCS(max)) the patent should received based on earlier modeling information. MCS(max) is shown and described in further detail with respect to FIG. 34A.

FIG. 34A shows operation of the neural dose adjustment interface 1020 in a first example. It is assumed that the stimulation parameters are adjustable in set increments: for example, that frequency is adjusted in 100 Hz increments (500, 600, 700, etc.); that pulse width is adjustable in 20 microsecond increments (140, 160, 180, etc.); and that amplitude is adjustable in 0.3 mA increments (e.g., 2.4, 2.7, 3.0 etc.). These increments may be set in accordance with the step size option selected (1022, FIG. 33D). Note that the GUI 1000 and IPG may be able to produce stimulation parameters different from these increments: for example, amplitude may generally be adjustable in 0.1 mA increments. Still, quantizing the stimulation parameters when interface 1020 is used can be helpful to allow the patient to more quickly arrive at stimulation parameters that are at least close to optimal to treat their symptoms. As explained further below, once interface 1020 is used to generally good stimulation parameters for the patient, the user can turn off the interface 1020 to allow stimulation parameters to be adjusted more finely.

Selection of the interface 1020 can include within GUI 1000 a graphic 1030 that provide the user some idea of how they are navigating the subset 425f. In the example shown, graphic 1030 comprises a graph of mean charge-per-second (MCS) versus frequency. Displaying information is this manner is useful because it gives the user a sense whether the total energy provided by the stimulation is relatively high or low at the current frequency. Note that this graphic 1030 can include an indication of maximum and minimum MCS values (MCS(max), MCS(min) at each frequency, giving the user some sense of the constraints imposed by the modeling (in this case, subset 425f). Note that the MCS(min) comprises the product of the frequency and the minimum pulse width and amplitude at that frequency (i.e., F*PW(min)*A(min)), while MCS(max) comprises the product of the frequency and the minimum pulse width and amplitude at that frequency (i.e., F*PW(max)*A(max)). The GUI 1000 may also show information 1040 as the interface 1020 is stepped through, including the stimulation parameter (A, PW, F) associated with each step, the resulting MCS value, and a percentage of the (maximum) MCS. As noted earlier, this percentage values can provide the patient with an indication whether the stimulation is relatively high or low given the modelling described earlier.

Generally speaking, the goal of interface 1020 is to step stimulation through different simulation parameters set within the subset 425f. In so doing, the stimulation is kept between MCS(max) and MCS(min) at each frequency. The interface 1020 can step through these parameter sets in different ways, but in one example the interface steps the stimulation parameters sets from a lowest MCS value (step 1) to a highest MCS value (step 95), as generally indicated by the arrow in graphic 1030 in FIG. 34A. FIG. 34B shows one example of how this can occur. At Step 1 a lowest frequency in subset 425f (500 Hz) is selected, as well as a lowest amplitude and pulse width. Per modeling (425f), the minimum amplitude at this frequency is (2.5 mA) and the minimum pulse width is 150 microseconds. However, because these values aren't consistent with the manner in which these parameters are quantized, they are rounded to 2.7 mA and 160 microseconds at Step 1 in FIG. 34B. Note that rounded up these values ensure the parameters applied to the patient at Step 1 are within the MCS limits.

When the step is incremented (Step 2), the amplitude is increased to its next quantized value (3.0 mA). This continues (Steps 3, 4) until the amplitude reaches its maximum (Step 5). This maximum amplitude per modeled subset 425f is 4.0 mA, and Step 5 a quantized value of 3.9 mA is used (4.2 mA would exceed the maximum). At this point, the pulse width can be incremented to its next quantized value (180 μs), with the amplitude set to its lowest value (2.7 mA) (Step 6). The amplitude can then be increased (Steps 7-9) until the maximum value is again reached (Step 10), at which point the pulse width can again be increased, and the amplitude again set to a lowest value (Step 11). This pattern continues until the amplitude and pulse width are maximized (3.9 mA and 200 μs) at the frequency in question (Step 15).

At this point (Step 16), the frequency is then incremented (to 600 Hz), and amplitude and pulse widths are set to minimum values at this new frequency. Referring again to subset 425f (FIG. 34B), the minimum amplitude and pulse width at F=600 Hz are 2.5 mA and 140. Thus, in Step 16 the amplitude is set to 2.7 mA (quantized, rounded up) and 140 (itself a quantized value). The above-described pattern is then repeated at this frequency by increasing the amplitude to its maximum, then increasing the pulse width and minimizing the amplitude, etc., until again a maximum amplitude and pulse width is reached (Step 35). The frequency is then incremented (to 700 Hz, Step 36), etc. As this pattern continues, eventually at Step 95 the maximum frequency (1000 Hz), and the maximum amplitude and pulse width at this frequency is reached.

To summarize, the interface 1020 is used to step though stimulation parameter sets in the subset 425f, with the hope of finding a set that best treats the patient. Assume for example, the patient finds Step 18 to provide the best relief (F=600 Hz, A=3.3 mA, PW=140 microseconds). At this point the user can deselect use of the interface 1020 and experiment further as necessary to “tweak” these stimulation parameters in the GUI 1000, effectively retuning to what was shown in FIG. 33D. At this point the user could increase a decrease the stimulation parameters more finely than allowed by the step size adjustment 1020 (e.g., in 0.1 mA increments, or 10 microsecond increments). In any event, optimal stimulation parameters within subset 425f are arrived at more easily and intuitively.

The graphic 1030 of FIG. 34A shows certain of the steps of FIG. 34B. Notice that as the interface 1020 and steps are incremented that the mean charge-per-second remains constrained between MCS(max) and MCS(min). Notice further that increasing steps do not necessarily monotonically increase the MCS. For example, Step 6 has a lower MCS value than Step 4. This is because, as shown in FIG. 34B, Step 6 has a large pulse width but a lower amplitude than Step 4. Of course, the manner in which interface 1020 navigates subset 425f is just one example, and the stimulation parameters sets could be navigated in different orders.

FIG. 34C shows another manner in which the interface 1020 can navigate the stimulation parameters in subset 425f. In this example, the MCS of the stimulation does monotonically increase with step value, starting at 216 μCis at Step 1, and steadily increasing as the steps are incremented. Essentially, one can understand FIG. 34C as the data of FIG. 34B sorted by increasing MCS values. To understand the differences between these approaches, the step numbers from FIG. 34B are included in FIG. 34C (e.g., what was Step 16 in FIG. 34B is now Step 2 in FIG. 34C). Organizing operation of the interface 1020 per FIG. 34C is beneficial because the neural dose of stimulation the patient receives is reliably increased or decreased, even if this results in seemingly random adjustment of the stimulation parameters. For example, in FIG. 34C notice that Step 8 specifies F=700 Hz, PW=140 μs, and A=2.7 mA, while next Step 9 specifies F=500 Hz, PW=180 μs, and A=3.0 mA.

While FIG. 34C shows the various steps/stimulation parameters ordered in accordance with their MCS values, they could be ordered and selected differently. For example, the steps could also be ordered by % max MCS, as described earlier.

Various aspects of the disclosed techniques, including processes implementable in the IPG or ETS, or in external devices such as the clinician programmer or external controller to render and operate the GUI 64, can be formulated and stored as instructions in a computer-readable media associated with such devices, such as in a magnetic, optical, or solid state memory. The computer-readable media with such stored instructions may also comprise a device readable by the clinician programmer or external controller, such as in a memory stick or a removable disk, and may reside elsewhere. For example, the computer-readable media may be associated with a server or any other computer device, thus allowing instructions to be downloaded to the clinician programmer system or external controller or to the IPG or ETS, via the Internet for example. Methods involving use of the disclosed subject matters also comprise aspects of Applicant's invention.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A system for controlling an implantable medical device of a patient, comprising:
a first external device for configured to program the implantable stimulator device, comprising:
a graphical user interface; and
control circuitry configured to:
receive a model comprising a plurality of sets of stimulation parameters developed for the patient, each of the sets of stimulation parameters being executable by the implantable stimulator device to provide stimulation to the patient,
provide on the graphical user interface an adjustment for each of the stimulation parameters, wherein a range within which each stimulation parameter can be adjusted is automatically constrained by the model, and
wherein when one of the stimulation parameters is adjusted within its range to a value, the control circuitry is configured to automatically constrain the ranges within which the other stimulation parameters can be adjusted in accordance with the model and the value.

2. The system of claim 1, wherein the first external device comprises a patient remote controller.

3. The system of claim 1, wherein the first external device comprises a clinician programmer.

4. The system of claim 1, wherein the stimulation parameters comprise a stimulation amplitude, pulse width, and frequency.

5. The system of claim 4, wherein the one of the stimulation parameters comprises the frequency, and wherein the other of the stimulation parameters comprises the pulse width and the amplitude.

6. The system of claim 1, further comprising a second external device, wherein the model is determined at the second external device and transmitted to the first external device.

7. The system of claim 1, wherein the control circuitry is further configured to provide on the graphical user interface an option to select a stimulation mode from a plurality of stimulation modes, wherein the range within which each stimulation parameter can be adjusted is automatically further constrained by the selected stimulation mode.

8. The system of claim 7, wherein at least one of the stimulation modes corresponds to an activity or posture of the patient.

9. The system of claim 7, wherein at least one of the stimulation modes corresponds to stimulation having a particular feeling for the patient.

10. The system of claim 7, wherein at least one of the stimulation modes comprises a low power mode.

11. A method for controlling an implantable medical device of a patient, comprising:
providing a model at a first external device, wherein the model comprises a plurality of sets of stimulation parameters developed for the patient, each of the sets of stimulation parameters being executable by the implantable stimulator device to provide stimulation to the patient;
providing on a graphical user interface of the first external device an adjustment for each of the stimulation parameters, wherein a range within which each stimulation parameter can be adjusted is automatically constrained by the model;
adjusting one of the stimulation parameters within its range to a value; and
automatically constraining the ranges within which the other stimulation parameters can be adjusted in accordance with the model and the value.

12. The method of claim 11, wherein the first external device comprises a patient remote controller.

13. The method of claim 11, wherein the second external device comprises a clinician programmer.

14. The method of claim 11, wherein the stimulation parameters comprise a stimulation amplitude, pulse width, and frequency.

15. The method of claim 14, wherein the one of the stimulation parameters comprises the frequency, and wherein the other of the stimulation parameters comprises the pulse width and the amplitude.

16. The method of claim 11, further comprising determining the model at a second external device and transmitting the model to the first external device.

17. The method of claim 11, wherein the graphical user interface further provides an option to select a stimulation mode from a plurality of stimulation modes, wherein the range within which each stimulation parameter can be adjusted is automatically further constrained by the selected stimulation mode.

18. The method of claim 17, wherein at least one of the stimulation modes corresponds to an activity or posture of the patient.

19. The method of claim 17, wherein at least one of the stimulation modes corresponds to stimulation having a particular feeling for the patient.

20. The method of claim 17, wherein at least one of the stimulation modes comprises a low power mode.
